(19) United States
(12) Patent Application Publication
(10) Pub. No.: US 2013/0149320 A1
(43) Pub. Date: Jun. 13, 2013

(54) Asf1b as a Prognosis Marker and Therapeutic Target in Human Cancer

(71) Applicants: Genevieve Almouzni, Neuilly Sur Seine (FR); Armelle Corpet, Paris (FR)

(72) Inventors: Genevieve Almouzni, Neuilly Sur Seine (FR); Armelle Corpet, Paris (FR)

(73) Assignees: CENTRE NATIONAL DE LA RECHERCHE SCIENTIFIQUE, PARIS (FR); INSTITUT CURIE, PARIS (FR)

(21) Appl. No.: 13/700,792

(22) PCT Filed: May 31, 2011
(86) PCT No.: PCT/EP2011/058939

§ 371 Date: Feb. 5, 2013

(30) Foreign Application Priority Data
May 31, 2010 (EP) 10164424.3

(51) Int. Cl. G01N 33/68; A61K 45/06; A61K 31/7088; A61K 38/17; C12Q 1/68; A61K 39/395

(52) U.S. Cl. CPC G01N 33/6875; C12Q 1/6886; A61K 39/39558; A61K 31/7088; A61K 38/17; A61K 45/06
USPC 424/174.1; 435/7.92; 435/6.12; 506/9; 514/44 A; 514/44 R; 514/19.3; 436/501

(57) ABSTRACT

The present invention provides a prognostic marker in human cancer, Asf1b, a high expression thereof being associated with a poor prognosis. The present invention also provides a method for selecting a subject affected with a cancer for an adjuvant therapy. Finally, the present invention provides a new therapeutic target for treating cancer.

### FIELD OF THE INVENTION

The present invention relates to the field of medicine, in particular of oncology. It provides a new prognostic marker in human cancer.

### BACKGROUND OF THE INVENTION

Cancer occurs when cell division gets out of control and results from impairment of a DNA repair pathway, the transformation of a normal gene into an oncogene or the malfunction of a tumor supressor gene. Many different forms of cancer exist. The incidence of these cancers varies but it represents the second highest cause of mortality, after heart disease, in most developed countries. While different forms of cancer have different properties, one factor which many cancers share is the ability to metastasize. Distant metastasis of all malignant tumors remains the primary cause of death in patients with the disease.

The therapeutic care of the patients having cancer is primarily based on surgery, radiotherapy and chemotherapy and the practitioner has to choose the most adapted therapeutic strategy for the patient. In the majority of the cases, the choice of the therapeutic protocol is based on the anatomo-pathological and clinical data. Currently, the methods to determine prognosis and select patients for adjuvant therapy rely mainly on pathological and clinical staging. However, it is very difficult to predict which localized tumor will eventuate in distant metastasis. Indeed, due to insufficiently accurate prognosis predictions, a substantial proportion of cancer subjects with inherently good outcome receive adjuvant systemic therapy without gaining any benefit.

Therefore, there is a great need for the identification of prognostic markers that can accurately distinguish tumors associated with poor prognosis including high probability of metastasis, early disease progression, increased disease recurrence or decreased patient survival, from the others. Using such markers, the practitioner can predict the patient's prognosis and can effectively target the individuals who would most likely benefit from adjuvant therapy.

The understanding of the molecular basis of cancer has advanced tremendously with the identification of mutations in the genome of tumor cells. Yet, while numerous studies support a major role for genetic events in cancer susceptibility, in particular for breast cancers, this genetic contribution alone does not explain the clinical complexity and heterogeneity of cancers, therefore suggesting that other mechanims may contribute to the process of tumorigenesis and aggressiveness. A current challenge is to find how, beyond genetic alterations, changes in the higher order nuclear organization of DNA into a complex chromatin structure participate in tumorigenesis. Abnormal gene expression (mostly gene silencing) in cancer cells associates with changes in DNA methylation and aberrant histone post-translational modifications in corresponding promoter regions. In addition, genome-wide changes of specific histone modifications are predictive of clinical outcome in specific cancers (Kurdistani, 2007). Thus, to consider how particular alterations in chromatin organization and histone dynamics occur in cancer and how they could promote tumorigenesis is of major interest.

Histone proteins form the core of the repeated unit of chromatin, the nucleosome, in which 146bp of DNA is wrapped around an octamer comprising (H3-H4-H2A-H2B)₂ histones. They are handled by histone chaperones (De Koning et al., 2007), which are critical for histone dynamics during DNA replication (Corpet and Almouzni, 2009; Ransom et al., 2010). Chromatin Assembly Factor 1 (CAF-1), a complex of three polypeptides RbAp48, p60 and p150 in mammals, is the primary histone chaperone involved in the deposition of H3-H4 histones coupled to DNA replication or repair (De Koning et al., 2007). Interestingly, CAF-1 p60 is a proliferation marker with diagnostic and prognostic value in breast cancer (Polo et al., 2004; Polo et al., 2010; Mascolo et al., 2010; Staibano et al., 2011). CAF-1 subunits as well as CAF-1 partners; Asf1a (Anti-silencing function 1), Asf1b, PCNA (Proliferating Cell Nuclear Antigen), and HP la (Heterochromatin protein 1) (see below) are more abundantly expressed in tumoral versus normal cells (WO 2005/085860; Polo et al., 2004).

In the present invention, the inventors focused on the histone H3-H4 chaperone Asf1. First identified by its ability to de-repress transcriptional silencing when overexpressed in yeast, Asf1 has been implicated in transcriptional regulation in yeast and Drosophila. Current data suggest a conserved role of Asf1 during DNA replication as a histone donor for CAF-1 which in turn deposits histones onto DNA. However, while a single iso form of Asf1 is present in yeast, higher order organisms such as plants, worms or mammals possess more than one gene encoding Asf1. Phylogenetic studies indicate that the distinction of two Asf1 iso forms, called Asf1a and Asf1b, is specific to mammals. Their highly conserved N-terminus provides a binding interface with the H3-H4 histones (De Koning et al., 2007), and their most divergent C-terminal part is less characterized (FIG. 1A). Both Asf1 iso forms can interact with p60, the mid-subunit of CAF-1. Collectively, the two human Asf1 isoforms have both been implicated in buffering the transient overload of replicative histone H3.1 that accumulates during replication stress, as well as in the control of S phase progression (Groth et al., 2005; Groth et al., 2007). Found in the nucleus, in complex with the MCM2-7 proteins, the putative helicase that unwinds DNA ahead of the replication fork, human Asf1a and Asf1b participate in regulating replication fork progression (Groth et al., 2007; Jasencakova et al., 2010). This is likely caused by defects in the transfer of parental histones. Notably, defects in S phase progression occurred only upon combined depletion of both Asf1 isoforms, indicating that Asf1a and Asf1b can substitute/compensate for one another in this function. However, the possible compensation of one iso form by the other does not exclude a regulation of task distribution under normal situations. The higher expression of Asf1b in human tissues such as thymus or testis does provide a first hint in this direction (Umehara and Horikoshi, 2003). Furthermore, human Asf1a proves most efficient to rescue defects in the DNA damage response in yeast depleted of endogenous Asf1, while human Asf1b compensates for the growth defects and the sensitivity to replicational stress (Tamburini et al., 2005). In mammalian cells, a specific interaction of Asf1a with HIRA is critical for senescence-associated cell cycle exit (Daganzo et al., 2003; Tang et al, 2006; Zhang et al., 2005). In addition, Asf1a has been involved in the regulation of H3K56 acetylation in human cells (Das et al., 2009; Yuan et al., 2009) which is a key histone mark during processes such as replication or repair. Taken together, while the distinct Asf1 iso forms share the common molecular properties enabling them to handle H3-H4 histone pools, how they exploit these properties in different context by involving a regulated distribution of tasks between the two Asf1 iso forms remained still poorly characterized.

DNA microarrays have compared normal cells to breast cancer cells and found differences in hundreds of genes, but the significance of most of those differences is unknown. Several screening tests are commercially marketed, but the evidence for their value is limited. The only test supported by Level II evidence is Oncotype DX, which is not approved by the U.S. Food and Drug Administration (FDA) but is endorsed by the American Society of Clinical Oncology. MammaPrint (van't Veer et al., 2002) is approved by the FDA but is only supported by Level III evidence. Two other tests have Level III evidence: Theros and MapQuant Dx. No tests have been verified by Level I evidence (in a prospective, randomized controlled trial, patients who used the test had a better outcome than those who did not). In a review, Sotirou concluded, “The genetic tests add modest prognostic information for patients with HER2-positive and triple-negative tumors, but when measures of clinical risk are equivocal (e.g., intermediate expression of ER and intermediate histologic grade), these assays could guide clinical decisions”. Various gene expression signatures including Asf1a or Asf1b for cancer prognosis have been disclosed. For instance in WO07/070621, a signature comprising at least 25 genes to be selected among a list of 100 genes (including Asf1b) for predicting solid tumor outcome has been disclosed, without any supporting data. Rosty et al., 2005 disclosed a cervical cancer proliferation gene cluster composed of 163 highly correlated transcripts among which Asf1b. In addition, Wirapati et al (2008) disclose a prognosis signature for breast cancer of 355 genes including Asf1b.

A comprehensive analysis of gene-expression-based classifiers recently compared nine gene expression signatures thought to be associated with breast cancer outcome (Regal et al., 2008). Overall, the nine signatures had similar performance in terms of assigning a sample to either a poor outcome group or a good outcome group. However, such signatures exhibited a large degree of discordance with 50% of the samples receiving at least one outcome assignment that is discordant with the assignments of the other signatures. In addition, no signature showed strong association with survival when applied to subgroups of tumor with aggressive feature i.e. lymph node positive, ER negative or high grade.

Therefore, due to the adverse effects of chemotherapy and radiotherapy, there is still a strong need to provide the most accurate prognosis as possible to avoid the unnecessary treatment of patients.

### SUMMARY OF THE INVENTION

The inventors demonstrated a clear association of human Asf1b, but not Asf1a, with proliferation capacity and the prognostic value of Asf1b in early stage cancer. Remarkably, high Asf1b expression levels significantly correlates with the tumor proliferation status, and with a poor prognosis outcome including the appearance of metastasis, increased disease recurrence, and the overall survival of the patients. Asf1b thus represents a new proliferation marker, which is relevant for the prognosis in cancer. In addition, inhibition of Asf1b expression was shown to decrease cell proliferation, highlighting the role of Asf1b as a new target for drug discovery in cancer or for treating cancer.

Accordingly, in a first aspect, the present invention concerns a method for predicting or monitoring clinical outcome of a subject affected with a cancer, wherein the method comprises the step of determining the expression level of Asf1b in a cancer sample from said subject, a high expression level of Asf1b being indicative of a poor prognosis. Preferably, a poor prognosis is a decreased patient survival and/or an early disease progression and/or an increased disease recurrence and/or an increased metastasis formation.

In a second aspect, the present invention concerns a method for selecting a subject affected with a cancer for a therapy, preferably an adjuvant therapy, or determining whether a subject affected with a cancer is susceptible to benefit from a therapy, preferably an adjuvant therapy, wherein the method comprises the step of determining the expression level of Asf1b in a cancer sample from said subject, a high expression level of Asf1b indicating that a therapy, preferably an adjuvant therapy, is required.

In a last aspect, the present invention concerns a method for monitoring the response to a treatment of a subject affected with a cancer, wherein the method comprises the step of determining the expression level of Asf1b in a cancer sample from said subject before the administration of the treatment, and in a cancer sample from said subject after the administration of the treatment, a decreased expression level of Asf1b in the sample obtained after the administration of the treatment indicating that the subject is responsive to the treatment.

In one embodiment, the expression level of Asf1b is determined by measuring the quantity of Asf1b protein or Asf1b mRNA.

In a further embodiment, the quantity of Asf1b protein is measured by immuno-histochemistry, semi-quantitative Western-blot or by protein or antibody arrays.

In another further embodiment, the quantity of Asf1b mRNA is measured by quantitative or semi-quantitative RT-PCR, or by real time quantitative or semi-quantitative RT-PCR or by transcriptome approaches.

In another embodiment, the methods of the invention comprise the step of comparing the expression level of Asf1b to a reference expression level. Additionally, the methods of the invention further comprise the step of determining whether the expression level of Asf1b is high compared to said reference expression level.

In a further embodiment, the reference expression level is the expression level of Asf1b in a normal sample.

Alternatively, the reference expression level is the expression level of a gene having a stable expression in different cancer samples, such as the RPLPO (ribosomal protein Po-like protein) gene.

In still another embodiment, the method according to the invention further comprises assessing at least another cancer or prognosis marker such as tumor grade, hormone receptor status, mitotic index, tumor size, HJURP (Holliday junction recognition protein, also called DKFZp762E1312, URLC9, hFLEG1, FAKTS) expression level or expression of proliferation markers such as Ki67 (antigen identified by monoclonal antibody Ki-67), MCM2 (minichromosome maintenance 2, also called D3S3194, KIAA0030, BM28, cdcl9), CAF-1 p60 (also called CAF1B or CHAF1B for Chromatin assembly factor 1, subunit B) and CAF-1 p150 (CAF1A or CHAF1A for Chromatin assembly factor 1, subunit A) or prognosis marker such as HP1α (heterochromatin protein 1-alpha, CBXS).

In addition, the present invention concerns the use of Asf1b as a prognosis marker in cancer, as a marker for selecting a subject affected with a cancer for a therapy, preferably an adjuvant therapy, or determining whether a subject affected with a cancer is susceptible to benefit from a therapy, preferably an adjuvant therapy, or as a marker for monitoring the response to a treatment of a subject affected with a cancer.

In still another embodiment, the cancer is a solid cancer or a hematopoietic cancer, preferably a solid cancer and more preferably, an early stage solid cancer without local or systemic invasion. Preferably, the cancer is selected from the group consisting of breast cancer, osteosarcoma, skin cancer, ovarian cancer, lung cancer, liver cancer, cervix cancer, liposarcoma, gastric cancer, pancreatic cancer, bladder cancer, vulvar cancer, colon cancer and brain cancer. More preferably, the cancer is breast cancer. Even more preferably, the cancer is an early stage breast cancer without local or systemic invasion.

The present invention also concerns a kit (a) for predicting or monitoring clinical outcome of a subject affected with a cancer; and/or (b) for selecting a subject affected with a cancer for a therapy, preferably an adjuvant therapy, or determining whether a subject affected with a cancer is susceptible to benefit from a therapy, preferably an adjuvant therapy;

and/or (c) for monitoring the response to a treatment of a subject affected with a cancer, wherein the kit comprises (i) at least one antibody specific to Asf1b; and/or (ii) at least one probe specific to the Asf1b mRNA or cDNA and/or (iii) at least one nucleic acid primer pair specific to Asf1b mRNA or cDNA and optionally, a leaflet providing guidelines to use such a kit. Preferably, the kit further comprises means for detecting the formation of the complex between Asf1b and said at least one antibody specific to Asf1b; and/or means for detecting the hybridization of said at least one probe specific to the Asf1b mRNA or cDNA on Asf1b mRNA or cDNA; and/or means for amplifying and/or detecting said Asf1b mRNA or cDNA.

The invention further concerns methods for selecting or identifying a molecule of interest capable of inhibiting Asf1b, preferably to selectively inhibit it. Therefore, the present invention concerns a method for selecting or identifying a molecule useful for the treatment of cancer, in particular for improving the clinical outcome of a patient having cancer, comprising testing a molecule for its ability to inhibit Asf1b and selecting the molecule capable of inhibiting Asf1b. Preferably, the ability of the test molecule to inhibit Asf1b is measured by a binding assay to Asf1b protein, by an assay measuring the Asf1b expression in a cell, by a nuclear morphology assay, by a Colony Formation Assay, by an in vitro nucleosome assembly assay or by a combination thereof.

Finally, the present invention concerns a molecule inhibiting Asf1b, preferably through direct interaction with Asf1b, preferably selectively in respect to Asf1a, for use in the treatment of cancer, in particular a cancer with a high expression level of Asf1b. Preferably, the molecule inhibiting Asf1b is selected from the group consisting of a small molecule, an aptamer against Asf1b, an antibody against Asf1b, a nucleic acid against Asf1b, a molecule preventing the interaction between Asf1b and one of its binding partners.

### BRIEF DESCRIPTION OF THE DRAWINGS

FIG. 1. Specificity of Asf1antibodies. FIG. 1A: (upper panel) Alignment of the two Asf1 isoforms, Asf1a (gi74735206) located on chromosome 6q22, and Asf1b (gi74734533) located on chromosome 19p13, was performed using ClustalW software (www.ebi.ac.uk/Tools/clustalw2/). Secondary structures in the conserved N-terminal region are indicated above the sequences. The C-terminus of Asf1a and Asf1b is more variable, harboring potential phosphorylation sites. Amino-acids that differ between Asf1a and Asf1b are in grey. Asterisks: identical residues, double dots: conserved residues; single dots: semi-conserved substitutions. (lower panel) Scheme depicting the percentage of homology between the different parts of Asf1a and Asf1b. Specific antibodies against Asf1a were raised against the full GST-Asf1a protein, whereas specific Asf1b antibodies were raised against a GST-C-Term-Asf1b (amino-acids 156 to 202). FIG. 1B: Western blot analysis of the two Asf1 antibodies on recombinant His-C-Terminal Asf1a and His-C-Terminal Asf1b showing a high specificity of these two antibodies on recombinant proteins. M: molecular weight marker. FIG. 1C: Western blot analysis of Asf1antibodies on total extracts from human U-2-OS cells, depleted of Asf1a, Asf1b, or Asf1a+b by siRNA for 48 h. Increasing amounts (x) of total cell extracts are loaded and α-tubulin serves as a loading control. While Asf1a antibody #28134 highly recognizes Asf1a and detects a faint band of Asf1b, Asf1b antibody #18143 is highly specific. M: molecular weight marker. FIG. 1D: IF analysis of U-2-OS cells treated as in (C) underscores the high specificity of the two Asf1 antibodies. Scale bar is 20 μm.

FIG. 2. Asf1a and Asf1b levels across the cell cycle in HeLa cells. FIG. 2A: Flow cytometry analysis of HeLa cells asynchronously growing (AS) or released from a double-thymidine block at the following times: Oh (G1/S), 4 h (S), 8 h (S/G2), and 14 h (G1). Mitotic cells (M) were collected after a 20 h nocodazole block. FIG. 2B: Western blot analysis of Asf1a and Asf1b levels in synchronized HeLa cells treated as in (A). Increasing amounts (x) of total cell extracts are loaded. Cyclin A, CAF-1 p60, and PCNA are shown for comparison. M: molecular weight marker. FIG. 2C: Quantitative RT-PCR analysis of Asf1a and Asf1b mRNA levels across the cell cycle in HeLa cells. Levels are normalized to the reference gene ribosomal protein Po-like protein (RPLPO) (de Cremoux et al., 2004). (a.u.): arbitrary units. Error bars represent data from two independent experiments.

FIG. 3. Expression of Asf1b depends on the cycling status of cells. FIG. 3A (left panel) Western blot analysis of total cell extracts from non treated asynchronous (AS) and quiescent (GO) MCF7 breast cancer cells and BJ primary foreskin fibroblasts. (right panel) Comparison of the behaviour of Asf1 isoforms in primary IMR90 human diploid fibroblasts young (PD27), old (PD72) and senescent (PD80). Increasing amounts (x) of total cell extracts are loaded. α-tubulin is a loading control. Asf1a and Asf1b have been revealed with a mix of the specific Asf1 antibodies (FIG. 1 and Table 3). CAF-1 p60 and Cyclin A have been used as markers for cell proliferation. M: molecular weight marker. (See also FIG. 2). FIG. 3B: Specific expression of Asf1a, Asf1b (FIG. 1) and the largest subunit of CAF-1 (p150) revealed by immunofluorescence in MCF7 cells asynchronous (AS) or quiescent (GO). DAPI stains nuclei. Scale bar is 20 μm. FIG. 3C: Flow cytometry analysis of the cell cycle distribution of MCF7 and BJ cells asynchronous (AS) or quiescent (GO). FIG. 3D: (left panel) Asf1a and Asf1b mRNA levels in proliferating (AS), quiescent (GO) and MCF7 cells and BJ primary fibroblasts as determined by Quantitative RT-PCR. (right panel) Asf1a and Asf1b mRNA levels in young, old or senescent IMR90 human diploid primary fibroblasts as determined by Quantitative RT-PCR. Levels have been normalized to the reference gene ribosomal protein Po-like protein (RPLPO) (de Cremoux et al., 2004) and levels in proliferating cells are set to 100%. The error bars represent s.d. from 2-3 independent experiments.

FIG. 4. Asf1b levels increase upon re-entry into the cell cycle. FIG. 4A Western blot analysis of total MCF7 breast cancer cell extracts from asynchronous (AS), quiescent (GO) or cells released from quiescence for the indicated times (2, 4, 8 and 24 hours). For increasing amounts (x) of total cell extracts, we revealed Asf1a and Asf1b with a mix of the specific Asf1 antibodies. α-tubulin was used as a loading control, CAF-1 p60, PCNA and Cyclin as markers for cell proliferation. M: molecular weight marker. FIG. 4B Asf1a and

Asf1b mRNA levels in proliferating (AS), quiescent (GO) and MCF7 cells released from GO are determined by Quantitative RT-PCR. Levels are normalized as in FIG. 3. The error bars represent s.d. from 3 independent experiments. FIG. 4C Specific expression of Asf1a, Asf1b and the largest subunit of CAF-1 (p150) revealed by immunofluorescence in MCF7 cells asynchronous (AS), quiescent (GO), or released from GO for the indicated times. DAPI stains nuclei. Scale bar is 20 μm. FIG. 4D Flow cytometry analysis of the cell cycle distribution of the cells shown in FIG. 4A.

FIG. 5. Asf1 expression levels in mammary cell lines. FIG. 5A Flow cytometry analysis of the breast cancer cell line Hs578T (T) and the non-tumoral mammary cell line

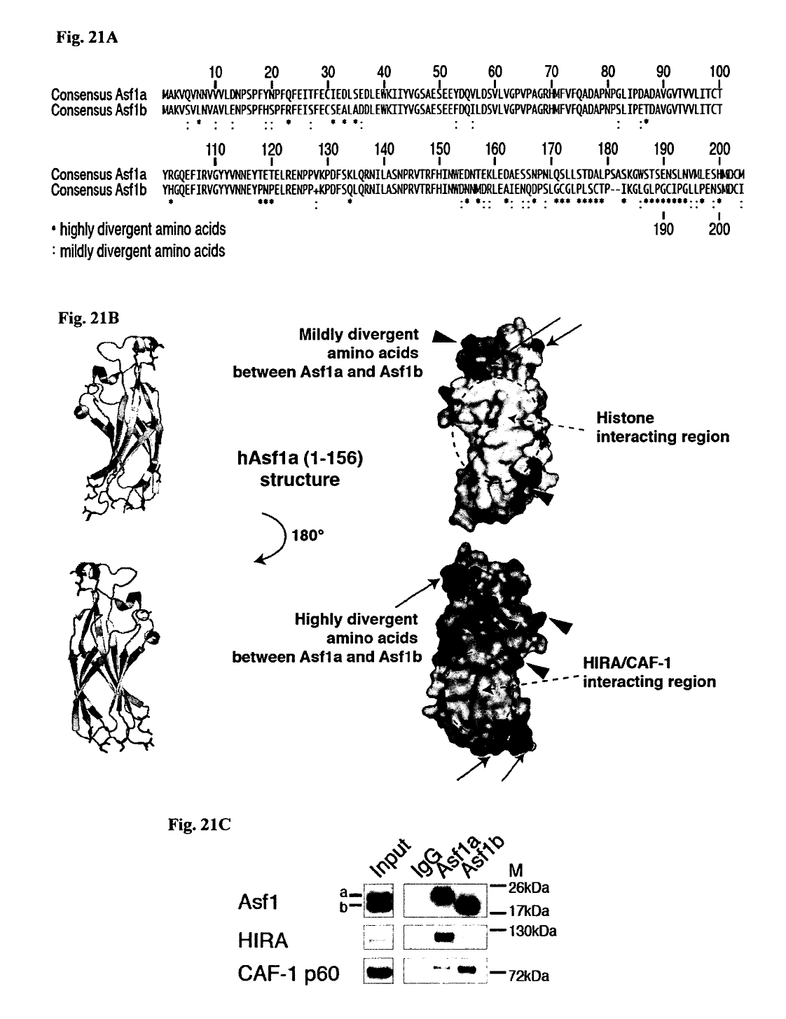

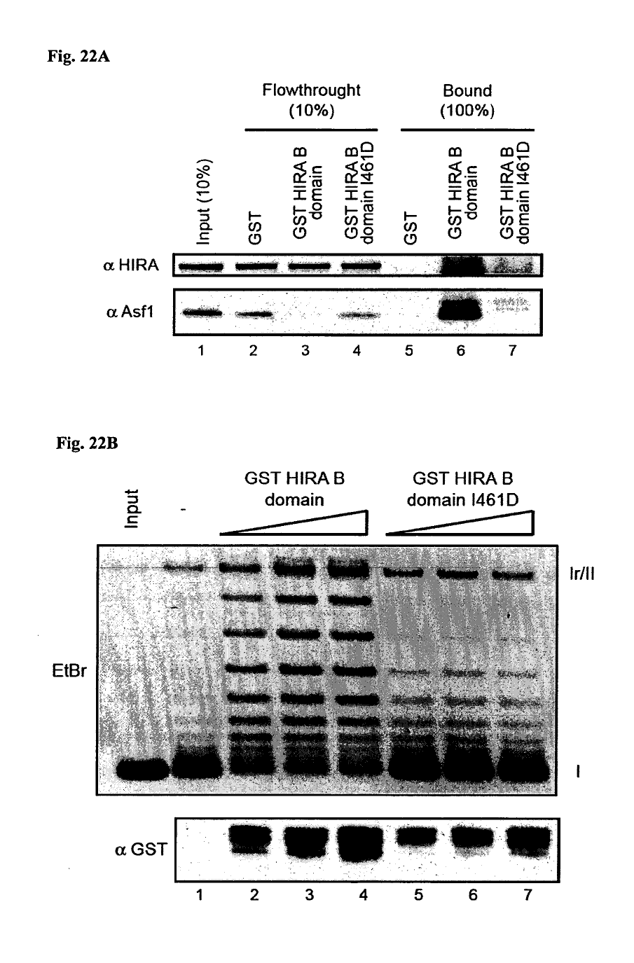

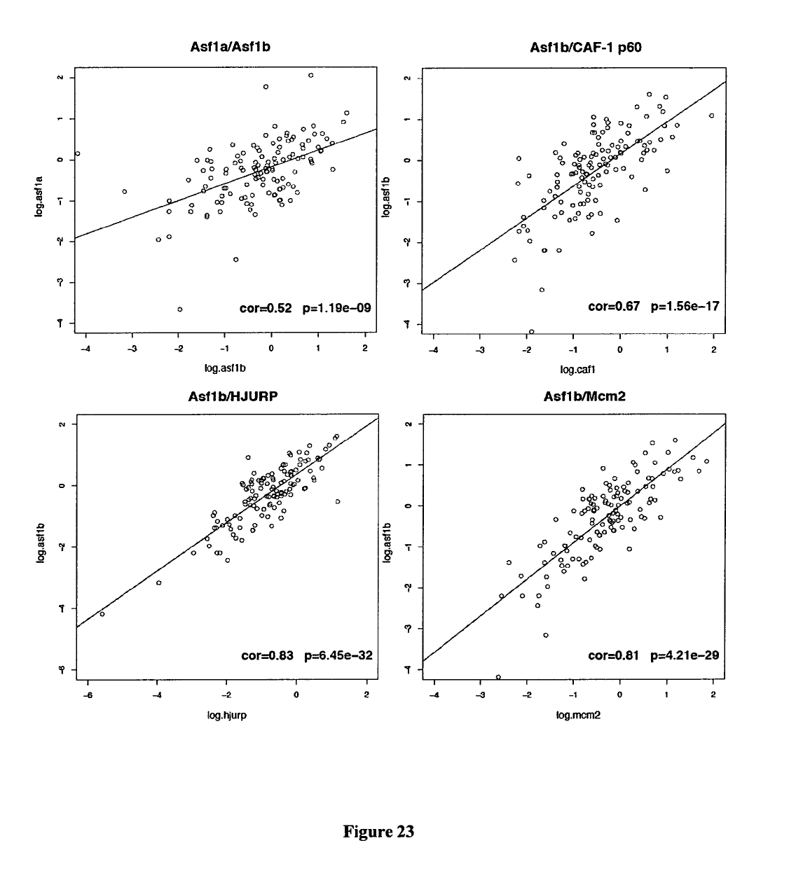

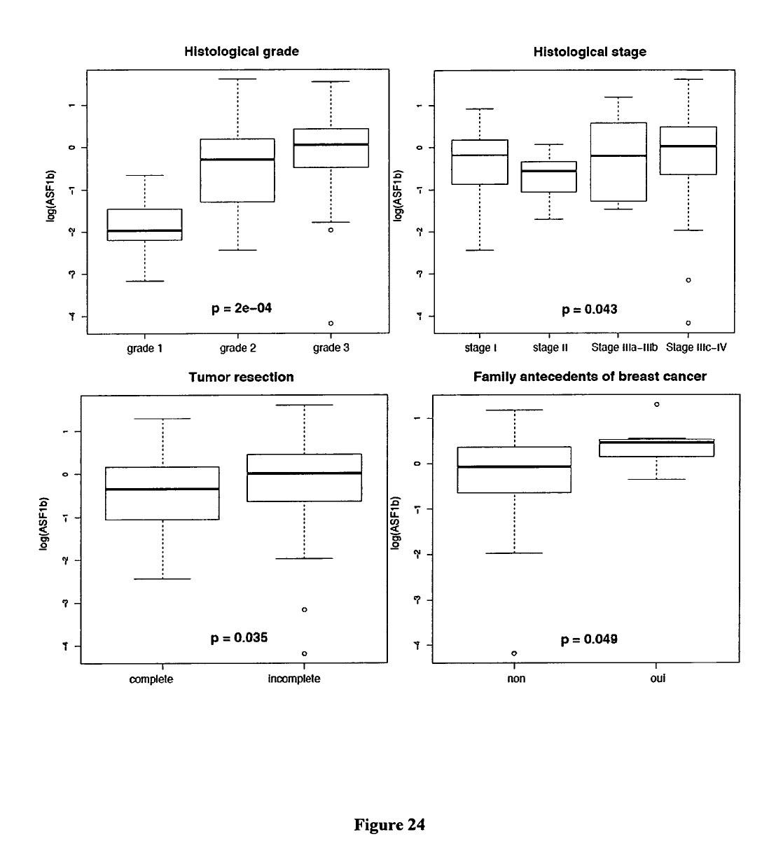

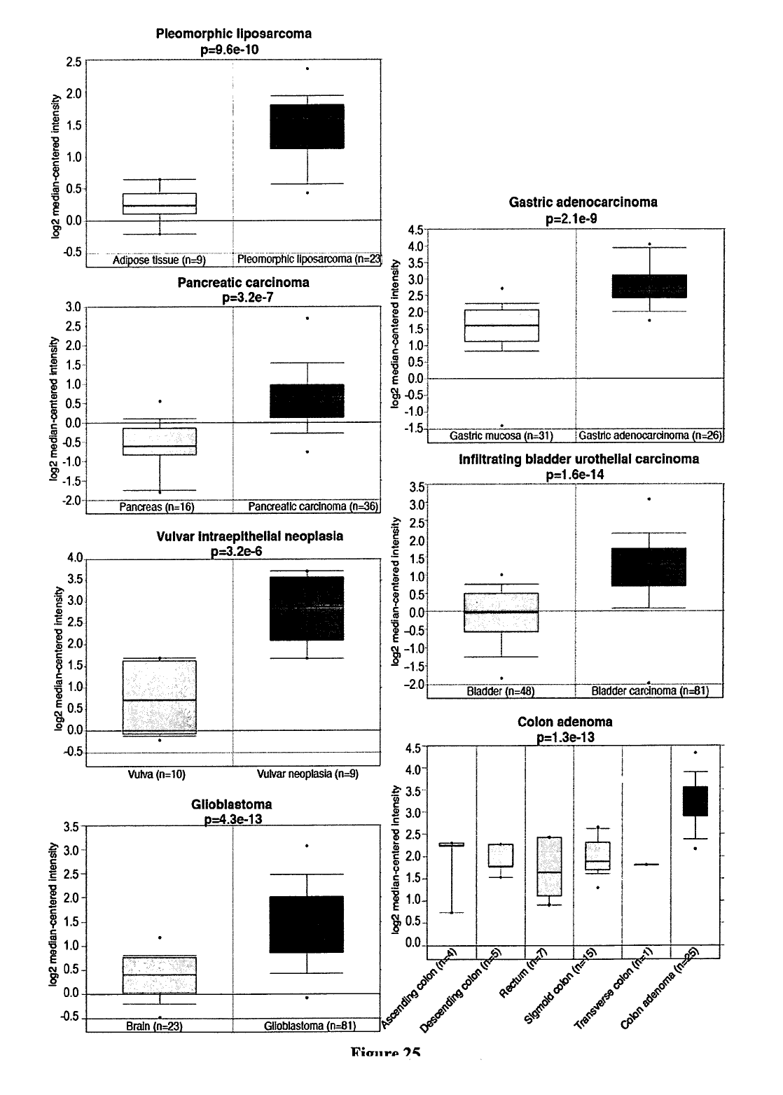

Hs578Bst (Bst), which are derived from the same patient, in order to assess polyploidy. Tumoral (T) and normal (Bst) cells contain 25% and 13% of cells in S phase respectively (De Koning et al., 2009). FIG. 5B Expression of Asf1a, Asf1b and CAF-1 p150 is revealed by immunofluorescence analysis of tumoral (T) and normal (Bst) mammary cell lines. Total levels of each protein are visualized as cells were not pre-extracted before fixation. Arrowheads indicate nuclei negative for CAF-1 p150 staining but positive for Asf1a staining Normal arrows point out nuclei double negative for CAF-1 p150 and Asf1b staining, underscoring that Asf1b levels parallels the proliferative status of the cells. Nuclei were stained with DAPI. Scale bar is 20 μm.

FIG. 6. Asf1b levels reflect the proliferating status of breast cell lines. FIG. 6A

Asf1a and Asf1b levels analyzed by Western blot analysis of total extracts from the tumoral (T) and the normal (Bst) mammary cell lines. Asf1(a+b) were revealed with a mix of the specific Asf1 antibodies and CAF-1 p60 is shown for comparison. Increasing amounts of cell extracts (x) are loaded. α-tubulin is a loading control. M: molecular weight marker. The percentage of cells in S phase (FIG. 5A) is indicated below the Western Blot. FIG. 6B Quantitative RT-PCR analysis of Asf1a and Asf1b mRNA levels in tumoral (T) and normal (Bst) mammary cell lines. Levels were normalized to the reference gene RPLPO and set to 100% in normal cells. Error bars represent s.d. from 3 independent experiments. FIG. 6C Specific expression of Asf1a, Asf1b and CAF-1 p150 revealed by immunofluorescence analysis of tumoral (T) and normal (Bst) mammary cell lines. Total levels of each protein are visualized as cells were not pre-extracted before fixation. Dapi stains nuclei. Scale bar is 10 μm. (See also FIG. 5B). FIG. 6D Pie charts show quantitative analysis of the proportion of cells in each of the categories indicated. ‘+’ indicates a positive staining while ‘−’ indicates a negative staining for the given protein. Black arc lines indicate an absence of correlation between Asf1a/b presence and CAF-1 p150 presence. Numbers represent the mean of 3 independent experiments (n=100 nuclei counted by experiment).

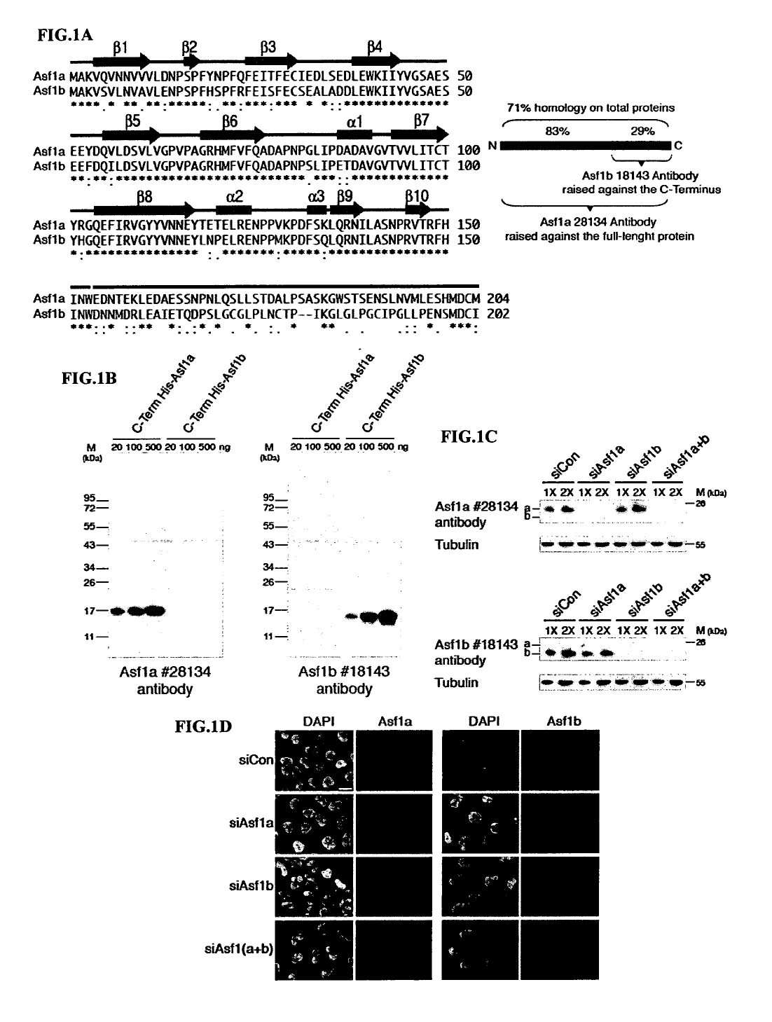

FIG. 7. Distinct effects of Asf1a and Asf1b depletions. FIG. 7A Specific depletion of Asf1 isoforms. (Left panel) Western blot analysis of total extracts from human U-2-OS cells showing the specific depletion of Asf1a, Asf1b or Asf1(a+b) for 48 h by siRNA treatment. Increasing amounts (x) of total cell extracts are loaded and α-tubulin serves as a loading control. A mix of the specific Asf1 antibodies (FIG. 1) reveals Asf1a and Asf1b. M: molecular weight marker. (Right panel) Flow cytometry analysis of the cell cycle distribution of the cells shown in the left panel. FIG. 7B Venn diagram showing the overlap between the significantly (p<0.05) differentially expressed genes determined in each siRNA condition indicated (siAsf1a, siAsf1b, and siAsf1(a+b)) versus the control siRNA. Numbers indicate the quantity of genes overlapping between 2 conditions. (See also FIGS. 8 and 9). FIG. 7C Colony Formation Assay for HeLa cells treated with the indicated siRNA. The mean surviving fraction (%) is indicated in the histograms below. Error bars represent data from 3 independent experiments. FIG. 7D Cellular defects upon specific depletion of Asf1 isoforms. Immunofluorescence analysis of U-2-OS cells treated as in (A). DAPI stains nuclei. Scale bar is 20 μm. FIG. 7E Histograms show quantitative analysis of the proportion of aberrant nuclear structures in U-2-OS cells treated as in (A). The mean percentage of altered nuclei (lobulated) and the percentage of micronucleated cells after 48 h of siRNA treatment are plotted. ±values indicate the standard deviation from counts of three and two independent experiments, respectively. FIG. 7F Immunofluorescence analysis of Lamin A staining in U-2-OS cells treated as in (A). DAPI stains nuclei. Scale bar is 10 _{R}m. FIG. 7G Colony Formation Assay for U-2-OS cells treated with two independent sets of siRNAs. The mean surviving fraction (%) is indicated in the histogramms. Error bars represent data from 2 independent experiments.

FIG. 8. Validation of transcriptomic data. FIG. 8A Quantitative RT-PCR analysis of Asf1a and Asf1b mRNA levels in U-2-OS cells depleted for Asf1a, Asf1b or Asf1(a+b) for 48 h by siRNA treatment. mRNA levels are normalized to the reference gene Glyceraldehyde 3-phosphate dehydrogenase (GAPDH) and expressed as the log2(fold change) relative to the control siRNA. The error bar represents data from three independent experiments. FIG. 8B mRNA extracted from human U-2-OS cells treated as in (A) were hybridized to Affymetrix HG-U133-Plus2 oligonucleotide microarrays. mRNA expression levels of Asf1a and Asf1b obtained from the Affymetrix hybridization are expressed as a log2(fold change) relative to the control siRNA depletion. Error bars represents data from two independent experiments. FIG. 8C Quantitative RT-PCR analysis of mRNA levels for the indicated genes in U-2-OS cells treated as in (A). mRNA levels are normalized to the reference gene GAPDH and expressed as the log2 fold change relative to the control siRNA. The error bar represents data from three independent experiments respectively. Below each graph is indicated the numerical value for the mean log2 fold change (FC) obtained by Q-RT-PCR or on the Affymetrix microarray.

FIG. 9. Gene Ontology (GO) analysis of differentially expressed genes after the specific knock-down of Asf1a or Asf1b or Asf1(a+b).

Gene Ontology (GO) annotation of genes were obtained on the microarray from the Ensemble database in March 2009. For each GO term, a Hypergeometric test was performed to determine whether genes of the differentially expressed list showed a more frequent association with a certain term than could be expected by chance given the GO annotation of all genes represented on the microarray. Histogram bars represent the -log(p-value) for each significant GO term. Terms for which the test resulted in a p-value of less or equal to 0.001 (-log(p-value) equal to 3 as shown by the dashed line) were considered to be significantly over-represented for the given list. Numbers in brackets indicate the number of significant genes found in the list of differentially expressed genes for the given GO category. Arrows indicate if the genes are up- or down-regulated in the indicated siRNA relative to the control siRNA.

FIG. 10. Asf1b, a proliferation marker with prognostic value in small breast cancers. FIG. 10A Correlations between the logarithmic mRNA expression levels of the indicated genes are depicted. The Pearson coefficient of correlation (r) and its associated p-value are indicated. Red numbers together with an asterisk * indicates a significant p-value (p<0.05). FIG. 10B Prognostic value of Asf1b in (T0/T1/T2-N0-M0) breast cancers. Univariate Kaplan-Meier curves of the survival in patients expressing low (Asf1b≦0.7) or high (Asf1b>0.7) levels of Asf1b. The number of patients at risk at each time point is indicated below each graphic. Significant p-values are ≦0.05.

FIG. 11. Asf1b levels correlate with proliferation and have a prognostic value in breast cancer patients. FIG. 11A Relative mRNA levels of Asf1a/b, p60 and Ki67 in (T0/T1/T2-N0-M0) breast cancers. Box plots representing logarithmic expression levels of Asf1a, Asf1b, CAF-1 p60 and Ki67 mRNAs, according to the indicated clinico-pathological factors in breast cancer samples with a >10 years patient follow-up. Boxes represent the 25th-75th percentile, brackets: range; black line: median; black dots: outliers. Below each graph the p-values determined by a Kruskal-Wallis' test are indicated. An asterisk * indicates a significant p-value (p<0.05). FIG. 11B Prognostic value of Asf1b in (T0/T1/T2-N0-M0) breast cancers. Univariate Kaplan-Meier curves of the disease free interval (interval before the occurrence of local recurrence, regional lymph node recurrence, contro-lateral breast cancer or metastasis), and the occurrence of metastasis (metastasis free interval) in patients expressing low (Asf1b≦0.7) or high (Asf1b>0.7) levels of Asf1b. An asterisk * indicates a significant p-value (p<0.05). The number of patients at risk at each time point is indicated below each graphic. (See also FIGS. 10 and 12).

FIG. 12. Asf1b correlates with prognosis in breast cancer and is overexpressed in multiple types of cancer. FIG. 12A Prognosis value in breast cancer. Boxplot representation of microarray expression levels of Asf1b in relation to prognosis in different breast cancer transcriptomes. Asf1b expression levels significantly correlate with the grade of the tumor (left), with the occurrence of metastasis at 5 years (middle) and with the disease free survival at 5 years (right). Results are analyzed and plotted using ONCOMINE database (Rhodes et al., 2004). Boxes represent the 25th-75th percentile, brackets: range; black line: median; black dots: outliers; n: sample number. p-values, based on Student's T-test, were considered as significant when p≦0.05. FIG. 12B Tumoral versus normal microarray expression data. Boxplot representation as in (A) of microarray expression levels of Asf1b in different types of cancer compared to normal tissue. Results from transcriptome studies on different tumor types are analyzed and plotted using ONCOMINE database (Rhodes et al., 2004). p-values, based on Student's T-test, were considered as significant when p≦0.05.

FIG. 13. Asf1b levels predict the occurrence of metastasis and distinguishes specific breast tumor subtypes. FIG. 13A Multivariate analysis adjusted for known prognostic factors (such as mitotic index (qualitative and quantitative), tumor size (qualitative and quantitative), tumor grade and Ki67 levels) and for our genes of interest (Asf1b, CAF-1 p60, CAF-1 p150 and HPlcc) in n=73 samples. Only CAF-1 p60 expression is an independent prognostic factor for the disease free interval and overall survival, while only Asf1b stands out as an independent prognostic factor for the occurrence of metastasis. In each case, the significant p-value (p<0.05), the Relative Risk (RR) and the 95% Confidence Interval (CI) is indicated. FIG. 13B Asf1a and Asf1b mRNA expression levels in different subtypes of breast cancer were analyzed from available transcriptomic data selected from the Institut Curie

Human Tumor database. BLC (Basal-Like Cancers) (n=17) and MBC (Medullary Basal-like Cancers) (n=19) are basal-like subtypes of breast tumors with MBC tumors characterized by an inflammatory stroma. LUMINAL (n=23) groups breast subtypes from the Luminal A and Luminal B category. MICRO (Micropapillary cancers) (n=22) indicates breast subtypes belonging to the Luminal B subtype. NORMAL (n=6) corresponds to normal breast tissue. Comparisons of the expression levels of Asf1a and Asf1b between sample groups were performed using two-sample Wilcoxon rank-sum tests. Boxes represent the 25th-75th percentile, brackets: range; black line: median; black dots: outliers. Significant p-values of these tests (<0.05) were corrected for multiple testing using the Bonferroni method. NS: Non-significant.

FIG. 14. Correlations of Asf1b with clinical data: set of patients from 1996. Box plots representing logarithmic expression levels of Asf1b mRNA according to the indicated clinicopathological factors in breast cancer samples with a >10 years follow-up. Boxes represent the 25th-75th percentile, brackets: range; black line: median; black dots: outliers. P-values determined by a Kruskal-Wallis' test are indicated. A significant p-value is <0.05.

FIG. 15. Prognostic value of Asf1b: set of patients from 1996. Univariate Kaplan-Meier curves of the metastasis free interval (interval before the occurrence of metastasis), the disease free interval (interval before the occurrence of local recurrence, regional lymph node recurrence, contro-lateral breast cancer or metastasis), and the disease free survival in patients expressing low (Asf1b <0.27) or high (Asf1>=0.27) levels of Asf1b. A significant p-value is p<0.05. The number of patients at risk at each time point is indicated below each graphic.

FIG. 16. Asf1b mRNA levels in different cell types compared to its levels in breast tumor samples from 1995.

The Asf1b mRNA levels were determined by quantitative RT-PCR in proliferating HeLa cells, in proliferating (AS) or quiescent (GO) MCF7 breast adenocarcinoma cells, in proliferating (AS) or quiescent (GO) BJ primary fibroblasts, in tumoral (Hst) and normal (Bst) mammary cell lines, and in breast tumor samples extracted in 1995 at Institut Curie. The same primers and amplification kit were used to detect Asf1b mRNA levels in cell lines or in breast tumor samples. Levels were normalized to the reference gene ribosomal protein Po-like protein (RPLPO) (de Cremoux et al., 2004). The cut-off value indicated corresponds to the cut-off defining two populations of patients: one with “low” Asf1b values which have a good prognosis, and another population of patients with “high” Asf1b values and which have a bad disease outcome. This cut-off value is consistent with Asf1b mRNA levels obtained in reference cell lines and may therefore be determined as a value which is above the levels of Asf1b mRNA in MCF-7 GO, BJ GO and/or Bst cell lines.

FIG. 17. Specific depletion of Asf1 isoforms in Hs578T cells. FIG. 17A. A. Western blot analysis of total extracts from human Hs578T cells showing the specific depletion of Asf1a, Asf1b or Asf1(a+b) for 48 h by siRNA treatment with two independent sets of oligonucleotides. Increasing amounts (x) of total cell extracts are loaded. α-tubulin serve as a loading control. Asf1a and Asf1b were revealed with a mix of the specific Asf1 antibodies. M: molecular weight marker. FIG. 17B Flow cytometry analysis of the cell cycle distribution of the cells shown in (A). Asf1b depletion slightly increases the number of cells in S/G2 phases. Asf1(a+b) depletion impairs S phase progression as demonstrated previously in U-2-OS cells (Groth et al., 2007). FIG. 17C Quantitative RT-PCR analysis of Asf1a and Asf1b mRNA levels in Hs578T cells treated as in (A). mRNA levels are normalized to the reference gene GAPDH and expressed as the log2(fold change) relative to the control siRNA. The error bar represents data from two independent experiments.

FIG. 18. Asf1b depletion impairs proliferation in Hs578T cells. FIG. 18A Immunofluorescence analysis of human Hs578T cells showing the specific depletion of Asf1a, Asf1b or Asf1 (a+b) for 48 h by RNA interference. DAPI stains nuclei. Scale bar is 10 μm. FIG. 18B Histograms show quantitative analysis of the proportion of aberrant nuclear structures in Hs578T cells treated as in (A). The mean percentage of altered nuclei (lobulated) and the percentage of micronucleated cells after 48 h of siRNA treatment are plotted. ±values indicate the standard deviation from counts of four independent experiments. FIG. 18C (left panel) Immunofluorescence analysis of Lamin A staining in Hs578T cells treated as in (A). Arrowheads marks DNA bridges. DAPI stains nuclei. Scale bar is 10 μm. (right panel) Histograms show quantitative analysis of the proportion of DNA bridges in Hs578T cells treated as in (A). ±values indicate the standard deviation from counts of two independent experiments. FIG. 18D Colony Formation Assay for Hs578T cells treated with two independent sets of siRNAs against Asf1 isoforms. The mean surviving fraction (%) is indicated in the histogramms. Error bars represent data from 3 independent experiments.

FIG. 19. Asf1b depletion impairs proliferation in MDA-MB-231 cells. FIG. 19A

Western blot analysis of total extracts from human MDA-MB-231 cells showing the specific depletion of Asf1a, Asf1b or Asf1 (a+b) for 48 h by siRNA treatment. Increasing amounts (x) of total cell extracts are loaded. α-tubulin serve as a loading control. Asf1a and Asf1b were revealed with a mix of the specific Asf1 antibodies. M: molecular weight marker. FIG. 19B Flow cytometry analysis of the cell cycle distribution of the cells shown in (A). FIG. 19C (left panel) Immunofluorescence analysis of human MDA-MB-231 cells treated as in (A). Arrowheads marks DNA bridges with micronuclei between cells. DAPI stains nuclei. Scale bar is 10 μm. (right panel) Histograms show quantitative analysis of the proportion of aberrant nuclear structures in MDA-MB-231 cells treated as in (A). The mean percentage of altered nuclei (lobulated) and the percentage of DNA bridges after 48 h of siRNA treatment are plotted. ±values indicate the standard deviation from counts of two independent experiments. FIG. 19D Colony Formation Assay for MDA-MB-231 cells treated with two independent sets of siRNAs against Asf1 isoforms. The mean surviving fraction (%) is indicated in the histogramms. Error bars represent data from 2 independent experiments.

FIG. 20. Multivariate analysis in patients of 1996. Multivariate analysis adjusted for known prognostic factors (such as mitotic index, tumor size, tumor grade and Ki67 levels) and for our genes of interest (Asf1b, CAF-1 p60, CAF-1 p150 and HP1α when significant in univariate analysis) in n=62 samples. This analysis performed on an independent set of patients confirms the prognostic value of Asf1b in predicting metastasis occurence. In each case, the significant p-value (p<0.05), the Relative Risk (RR) and the 95% Confidence Interval (CI) are indicated.

FIG. 21: Structural divergences between Asf1a and Asf1b isoforms in Amniotes. FIG. 21A Consensus sequences of Amniota Asf1a and Asf1b were determined based on their alignment in Jalview. Asterisks (*) indicate amino acids with a strong divergence while double dots (:) indicate a substitution of amino acids with a mild effect (conservation of the property of the amino acids). Divergent amino acids from the N-terminal region were reported on the structure of human Asf1a. FIG. 21B Structural surface view (left) and ribbon view (right) of human Asf1a N-terminus (aa 1-155) determined by NMR (PDB accession number 1TEY.pdb) (Mousson et al., 2005) and visualized using PYMOL software.

White surfaces indicate the histone interacting region determined as the amino-acids that are less than 4A in distance from H3-H4 in the crystal structure of Asf1-H3-H4 (PDB accession number 2I05.pdb) (Natsume et al., 2007). Light grey surfaces indicate the HIRA/CAF-1 p60 interacting region determined as the amino acids that are less than 4A in distance from the B-domain in the crystal structure of Asf1-B-domain of HIRA (PDB accession number 2I32.pdb) (Tang et al., 2006). Dark grey and black surfaces indicate the divergent amino-acids between Asf1a and Asf1b as determined by a comparison of the consensus sequence obtained for each Asf1 protein through the multiple alignment of vertebrate Asf1a and Asf1b protein sequences (see A). Dark grey surfaces (marked with an arrowhead) depict relatively mild amino-acids changes while black surfaces (marked with an arrow) show highly divergent amino-acids. FIG. 21C Immunoprecipitation performed on total cell extracts from human Hela S3 cells with either a control rabbit IgG antibody or the purified specific antibodies against Asf1 isoforms. Input is 5% of the immunoprecipitated material. Asf1a and Asf1b were revealed with a mix of the specific Asf1 antibodies, CAF-1 p60 and HIRA by western blotting. M: molecular weight marker. The inventors hypothesize that the divergent regions between Asf1a and Asf1b uncovered in (B) could mediate the preferential interaction of Asf1a with HIRA and Asf1b with CAF-1 p60.

FIG. 22. Inhibition of chromatin assembly with the B-domain of HIRA. FIG. 22A Binding of the wild-type but not the mutated form (I461D) of the B-domain of HIRA to Asf1. GST pull-down was performed on HSE extracts with 3 mg of recombinant GST B-domain of HIRA proteins on beads. Input and flowthrough are 10% of the immunoprecipitated material. Asf1 and HIRA were revealed by western blotting. FIG. 22B The B-domain of HIRA inhibits nucleosome assembly independent of replication. In vitro nucleosome assembly reactions independent of DNA synthesis were performed with increasing amounts (2, 4 or 8 mg) of the recombinant GST HIRA B-domain or its mutated form (I461D) added directly into the nucleosome assembly reaction mix. The purified plasmid DNA was analyzed by agarose elecrophoresis and was visualized by staining with ethidium bromide (EtBr). The migration positions of DNA plasmid form 1 (supercoiled), form 11 (nicked circular) and lr (closed circular) are indicated. Below the EtBr gel, GST proteins were revealed by Western Blotting.

FIG. 23. Asf1b levels strongly correlate with CAF-1p60, HJURP and Mcm2 levels. Correlations between the logarithmic mRNA expression levels of the indicated genes are depicted. The Pearson coefficient of correlation (r) and its associated p-value are indicated. Significant p-values (p<0.05) are indicated.

FIG. 24. Asf1b correlates with histological grade and stage in ovarian cancer. Box plots representing logarithmic expression levels of Asf1b mRNA according to the indicated clinico-pathological factors in ovarian cancer samples. Boxes represent the 25th-75th percentile, brackets: range; black line: median; black dots: outliers. Below each graph the p-values determined by a Kruskal-Wallis' test are indicated. Significant p-values (p<0.05) are indicated.

FIG. 25: Asf1b is overexpressed in a variety of cancers. Boxplot representation of microarray expression levels of Asf1b in different types of cancer compared to normal tissue. Results from transcriptome studies on different tumor types (Barretina et al., 2010; D'Errico et al., 2009; Pei et al., 2009; Sabates-Bellver et al., 2007; Sanchez-Carbayo et al., 2006; Santegoets et al., 2007; Sun et al., 2006) are analyzed and plotted using ONCOMINE database (Rhodes et al., 2004). p-values, based on Student's T-test, were considered as significant when p≦0.05. Boxes represent the 25th-75th percentile, brackets: range; black line: median; black dots: outliers; n: sample number.

### DETAILED DESCRIPTION OF THE INVENTION

Recent data have uncovered a number of alterations in chromatin organization in the context of cancer. How they occur and connect with other genetic alterations during the development of this disease is currently a major issue. In this context, understanding the role of histone dynamics represents a significant step forward which can help to classify cancer types. This is particularly important when considering the complexity and heterogeneity encountered in breast cancers.

Mammalian cells possess two closely related isoforms of the histone H3-H4 chaperone Anti-silencing function 1 (Asf1), Asf1a and Asfb. Asf1a and Asf1b are two isoforms encoded by two distinct genes. Up to date, their specific or redundant functions have remained elusive.

In the present study, the inventors aimed at unravelling the respective importance of the two isoforms of the histone chaperone Asf1, Asf1a and Asf1b, in relation to proliferation and tumorigenesis. Indeed, they reveal a clear association of human Asf1b, but not Asf1a, with proliferation capacity in cultured cells which proves relevant in breast tumors. Furthermore, they demonstrate the prognostic value of Asf1b in early stage breast cancer. Together, their findings highlight distinct functions for Asf1a and Asf1b and identify a novel molecular target for the development of new therapeutic strategies.

### Definitions

As used herein, the term “Asf1b” refers to the Anti-Silencing Function 1 homolog B or CIA-II. Unigene Cluster for Asf1b is Hs.26516. Representative mRNA and protein sequences are NM_{—}018154.2 or AF279307, and NP_{—}060624.1, respectively.

The term “cancer” or “tumor”, as used herein, refers to the presence of cells possessing characteristics typical of cancer-causing cells, such as uncontrolled proliferation, and/or immortality, and/or metastatic potential, and/or rapid growth and/or proliferation rate, and/or certain characteristic morphological features. This term refers to any type of malignancy (primary or metastases) in any type of subject, male of female. In particular, the term encompasses breast cancer at any stage of progression, such as “T” (primary), “N” (cancer has spread to lymph nodes) or “M” (cancer has metastasized). Typical cancers are solid or hematopoietic cancers such as breast, stomach, oesophageal, sarcoma, ovarian, endometrium, bladder, cervix uteri, rectum, colon, lung or ORL cancers, paediatric tumors (neuroblastoma, glyoblastoma multiforme), lymphoma, leukaemia, myeloma, seminoma, Hodgkin and malignant hemopathies. Preferably, the cancer is selected from the group consisting of breast cancer, osteosarcoma, skin cancer, ovarian cancer, lung cancer, liver cancer, cervix cancer, liposarcoma, gastric cancer, pancreatic cancer, bladder cancer, vulvar cancer, colon cancer and brain cancer. Preferably the cancer is a solid cancer, more preferably an early stage solid cancer without local or systemic invasion. Preferably, the solid cancer is breast cancer, more preferably an early stage breast cancer without local or systemic invasion.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease.

The term “therapy”, as used herein, refers to any type of treatment of cancer (i.e., antitumoral therapy), including an adjuvant therapy and a neoadjuvant therapy. Therapy comprises radiotherapy and therapies, preferably systemic therapies such as hormone therapy, chemotherapy, immunotherapy and monoclonal antibody therapy.

The term “adjuvant therapy”, as used herein, refers to any type of treatment of cancer given as additional treatment, usually after surgical resection of the primary tumor, in a patient affected with a cancer that is at risk of metastasizing and/or likely to recur. The aim of such an adjuvant treatment is to improve the prognosis. Adjuvant therapies comprise radiotherapy and therapy, preferably systemic therapy, such as hormone therapy, chemotherapy, immunotherapy and monoclonal antibody therapy.

The term “neoadjuvant therapy”, as used herein, refers to any type of treatment of cancer given prior to surgical resection of the primary tumor, in a patient affected with a cancer. The most common reason for neoadjuvant therapy is to reduce the size of the tumor so as to facilitate a more effective surgery. Neoadjuvant therapies comprise radiotherapy and therapy, preferably systemic therapy, such as hormone therapy, chemotherapy, immunotherapy and monoclonal antibody therapy.

As used herein, the term “chemotherapeutic treatment” or “chemotherapy” refers to a cancer therapeutic treatment using chemical or biochemical substances, in particular using one or several antineoplastic agents.

The term “radiotherapeutic treatment” or “radiotherapy” is a term commonly used in the art to refer to multiple types of radiation therapy including internal and external radiation therapies or radioimmunotherapy, and the use of various types of radiations including X-rays, gamma rays, alpha particles, beta particles, photons, electrons, neutrons, radioisotopes, and other forms of ionizing radiations.

The term “immunotherapy” refers to a cancer therapeutic treatment using the immune system to reject cancer. The therapeutic treatment stimulates the patient's immune system to attack the malignant tumor cells. It includes immunization of the patient with tumoral antigens (eg. by administering a cancer vaccine), in which case the patient's own immune system is trained to recognize tumor cells as targets to be destroyed, or administration of molecules stimulating the immune system such as cytokines, or administration of therapeutic antibodies as drugs, in which case the patient's immune system is recruited to destroy tumor cells by the therapeutic antibodies. In particular, antibodies are directed against specific antigens such as the unusual antigens that are presented on the surfaces of tumors. As illustrating example, one can cite Trastuzumab or Herceptin antibody which is directed against HER2 and approved by FDA for treating breast cancer.

The term “monoclonal antibody therapy” refers to any antibody that functions to deplete tumor cells in a patient. In particular, therapeutic antibodies specifically bind to antigens present on the surface of the tumor cells, e.g. tumor specific antigens present predominantly or exclusively on tumor cells. Alternatively, therapeutic antibodies may also prevent tumor growth by blocking specific cell receptors.

The term “hormone therapy” or “hormonal therapy” refers to a cancer treatment having for purpose to block, add or remove hormones. For instance, in breast cancer, the female hormones estrogen and progesterone can promote the growth of some breast cancer cells. So in these patients, hormone therapy is given to block estrogen and a non-exhaustive list commonly used drugs includes: Tamoxifen, Fareston, Arimidex, Aromasin, Femara, Zoladex/Lupron, Megace, and Halotestin.

As used herein, the term “poor prognosis” refers to a decreased patient survival and/or an early disease progression and/or an increased disease recurrence and/or an increased metastasis formation.

As used herein, the term “subject” or “patient” refers to an animal, preferably to a mammal, even more preferably to a human, including adult, child and human at the prenatal stage. However, the term “subject” can also refer to non-human animals, in particular mammals such as dogs, cats, horses, cows, pigs, sheep and non-human primates, among others, that are in need of treatment.

The term “sample”, as used herein, means any sample containing cells derived from a subject, preferably a sample which contains nucleic acids. Examples of such samples include fluids such as blood, plasma, saliva, urine and seminal fluid samples as well as biopsies, organs, tissues or cell samples. The sample may be treated prior to its use. The term “cancer sample” refers to any sample containing tumoral cells derived from a patient, preferably a sample which contains nucleic acids. Preferably, the sample contains only tumoral cells. The term “normal sample” refers to any sample which does not contain any tumoral cells.

The methods of the invention as disclosed below, may be in vivo, ex vivo or in vitro methods, preferably in vitro methods.

The present invention relates to a method for predicting or monitoring clinical outcome of a subject affected with a cancer, wherein the method comprises the step of determining the expression level of Asf1b in a cancer sample from said subject, a high expression level of Asf1b being indicative of a poor prognosis. It is important to note that the expression level of Asf1b is a significant prognosis marker for clinical outcome taken alone. In addition, Asf1b has proven to be a significant prognostic marker that can be used for all types of breast cancer tumors tested including aggressive breast cancer tumors such as the basal-like subtype and the Erbb2 positive subtype.

In a further aspect, the present invention concerns a method for selecting a subject affected with a cancer for a therapy, preferably an adjuvant therapy, or determining whether a subject affected with a cancer is susceptible to benefit from a therapy, preferably an adjuvant therapy, wherein the method comprises the step of determining the expression level of Asf1b in a cancer sample from said subject, a high expression level of Asf1b indicating that a therapy, preferably an adjuvant therapy, is required. A high expression level of Asf1b indicates a decreased patient survival and/or an early disease progression and/or an increased disease recurrence and/or an increased metastasis formation. Accordingly, this type of cancer associated with poor prognosis has to be treated with a therapy, preferably an adjuvant therapy in order to improve the patient's chance for survival. The type of therapy is chosen by the practitioner. It includes radiotherapy, chemotherapy, hormonal therapy, immunotherapy and monoclonal antibody therapy. However, these therapies are usually aggressive and cause several side effects. By using the method according to the invention it is therefore possible to limit adjuvant therapy to subjects who really need them and spare a large subgroup of subjects (those identified as having a good prognosis) of a harmful, useless and expensive treatment.

In a last aspect, the present invention further concerns a method for monitoring the response to a treatment of a subject affected with a cancer, wherein the method comprises the step of determining the expression level of Asf1b in a cancer sample from said subject before the administration of the treatment, and in a cancer sample from said subject after the administration of the treatment, a decreased expression level of Asf1b in the sample obtained after the administration of the treatment indicating that the subject is responsive to the treatment. The treatment may be any type of treatment such as chemotherapy, hormone therapy, immunotherapy, monoclonal antibody, radiotherapy or any other type of cancer treatment. A first cancer sample is obtained from the subject before the administration of the treatment. A second sample is obtained from the same subject after the administration of the treatment. Preferably, the second sample is collected when a significant effect on cell proliferation can be expected, dependent on the chosen treatment. In an embodiment, the second sample is collected at least 2 days after administration of the treatment, preferably one week after said administration. The responsiveness of the patient to the treatment is evaluated by determining the expression level of Asf1b in tumoral cells before and after the treatment. The expression level of Asf1b in the cancer sample is determined as described above, preferably by measuring the quantity of Asf1b protein or Asf1b mRNA. A lower expression level of Asf1b in tumoral cells contained in the sample collected after the treatment in comparison with the expression level of Asf1b in tumoral cells contained in the sample collected before the treatment indicates that the subject is responsive to the treatment. This method may also provide an indication of the required duration and/or intensity of the treatment. In this case, a follow-up after each cycle of treatment permits to determine if the expression level of Asf1b is lower than before the treatment, and thus to adjust the treatment duration and/or intensity accordingly. In particular, this method could be indicative of the efficacy of the treatment for increasing the disease free interval and/or, the overall survival, and/or for decreasing the metastasis occurrence.

In an embodiment of these above mentioned methods, the method further comprises the step of providing a cancer sample from the subject.

The expression level of Asf1b can be determined from a cancer sample by a variety of techniques. In an embodiment, the expression level of Asf1b is determined by measuring the quantity of Asf1b protein or Asf1b mRNA.

In a particular embodiment of these above mentioned methods, the expression level of Asf1b is determined by measuring the quantity of Asf1b protein. The quantity of Asf1b protein may be measured by any methods known by the skilled person. Usually, these methods comprise contacting the sample with a binding partner capable of selectively interacting with the Asf1b protein present in the sample. The binding partner is generally a polyclonal or monoclonal antibody, preferably monoclonal. Polyclonal and monoclonal antibodies anti-Asf1b are commercially available. Examples of marketed antibodies are the rabbit monoclonal anti-Asf1b antibodies from Cell Signaling Technology (#2902, #2769, produced by immunizing animals with a synthetic peptide corresponding to the carboxy-terminus of the human Asf1b protein) and the mouse monoclonal anti-Asf1b from Abnova (#H00055723-M01 and # H00055723-M03). In some embodiments, such as for immunofluorescence and immunohistochemistry applications, the antibody is specific to Asf1b compared to Asf1a, i.e. the antibody specific to Asf1b does not cross-react with Asf1b. For example, the rabbit monoclonal anti-Asf1b antibodies from Cell Signaling Technology (#2902, #2769) have been proved to be specific by the inventors. However, other antibodies such as Abnova #H00055723-M01 and #H00055723-M03 could also be tested for their specificity to Asf1b and their cross-reactivity with Asf1a. Other antibodies which can be used in the different methods to quantify the Asf1b protein are well known by the skilled person and are commercially available. In addition, the methods for producing anti-Asf1b antibodies are well-known in the art. In a preferred embodiment, the antibody is specific to Asf1b in comparison to Asf1a, i.e. the antibody specific to Asf1b does not cross-react with Asf1a (e.g., #2902, #2769 of Cell Signaling Technology). An antibody specific for Asf1b compared to Asf1a may be prepared by using either the C-terminal part of the protein where the sequence identity is lower and/or a segment including several amino acid substitutions (see FIG. 1A). However, due to their distinct migration on SDS-PAGE, Asf1a and Asf1b can be distinguished even with a cross-reacting antibody. The quantity of Asf1b protein may be measured by semi-quantitative Western blots, enzyme-labeled and mediated immunoassays, such as ELISAs, biotin/avidin type assays, radioimmunoassay, immunoelectrophoresis or immunoprecipitation or by protein or antibody arrays. The protein expression level may be assessed by immunohistochemistry on a tissue section of the cancer sample (e.g. frozen or formalin-fixed paraffin embedded material). The reactions generally include revealing labels such as fluorescent, chemiluminescent, radioactive, enzymatic labels or dye molecules, or other methods for detecting the formation of a complex between the antigen and the antibody or antibodies reacted therewith. Preferably, the quantity of Asf1b protein is measured by immunohistochemistry or semi-quantitative western-blot. Asf1b detection by immunohistochemistry does not require an antigen-unmasking step, unlike the routinely used marker Ki-67, and thus speeds up the staining process.

In another embodiment of these above mentioned methods, the expression level of Asf1b is determined by measuring the quantity of Asf1b mRNA. Methods for determining the quantity of mRNA are well known in the art. For example, the nucleic acid contained in the sample (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous. Preferably, primer pairs were designed in order to overlap an intron, so as to distinguish cDNA amplification from putative genomic contamination. An example of primer pair which may be used in this method is presented in the experimental section and is constituted by the primers of SEQ ID Nos 3 and 4. Other primers may be easily designed by the skilled person. Other methods of Amplification include ligase chain reaction (LCR), transcription-mediated amplification

(TMA), strand displacement amplification (SDA) and nucleic acid sequence based amplification (NASBA). Preferably, the quantity of Asf1b mRNA is measured by quantitative or semi-quantitative RT-PCR or by real-time quantitative or semi-quantitative RT-PCR or by transcriptome approaches.

In an embodiment of these above mentioned methods, the method further comprises the step of comparing the expression level of Asf1b to a reference expression level.

In particular, the reference expression level can be the expression level of Asf1b in a normal sample. The normal sample is a non-tumoral sample, preferably from the same tissue than the cancer sample. The normal sample may be obtained from the subject affected with the cancer or from another subject, preferably a normal or healthy subject, i.e. a subject who does not suffer from a cancer. Preferably, the normal sample is obtained from the same subject than the cancer sample. Expression levels obtained from cancer and normal samples may be normalized by using expression levels of proteins which are known to have stable expression such as RPLPO (acidic ribosomal phosphoprotein PO), TBP (TATA box binding protein), GAPDH (glyceraldehyde 3-phosphate dehydrogenase) or β-actin. In particular, expression of RPLPO may be assayed by Q-PCR with the following primers set: SEQ ID Nos 5 and 6.

Alternatively, the reference expression level may be the expression level of a gene having a stable expression in different cancer samples. Such genes include for example, RPLPO, TBP, GAPDH or β-actin. Preferably, the reference expression level is the expression level of the RPLPO gene. The use of the human acidic ribosomal phosphoprotein PO (RPLPO) gene as reference was described in the article of de Cremoux et al. (de Cremoux et al., 2004). In a preferred embodiment, the quantity of Asf1b mRNA is normalized according to the quantity of RPLPO mRNA. The quantity of RPLPO mRNA is used as reference quantity (i.e. 100%). The quantity of Asf1b mRNA is expressed as a relative quantity with respect to the quantity of RPLPO mRNA.

Expression of RPLPO may be assayed by Q-PCR with the following primers set: SEQ ID Nos 5 and 6 and expression of GAPDH may be assayed by Q-PCR with the following primers set: SEQ ID Nos 21 and 22. Of course, alternative primers set may be designed by the skilled in the art.

In a further embodiment of these above mentioned methods, the method further comprises the step of determining whether the expression level of Asf1b is high compared to the reference expression level.

The expression level of Asf1b in the cancer sample is considered as significantly different (i.e. high) compared to the reference expression level in a normal sample, if, after normalization, difference is in the order of 2.5-fold higher than the expression level in the normal sample or more. Preferably, the expression level of Asf1b in the cancer sample is considered as high if the level is at least 3-fold higher, or 4, 5 or 6-fold higher, than the expression level in the normal sample.

If the reference expression level is the expression level of a gene having a stable expression in different cancer samples, in particular the expression level of the RPLPO gene, the expression level of Asf1b is considered as high if the level or quantity of Asf1b mRNA is of at least or about 0.2, 0.25, 0.3, 0.35, 0.4, 0.45, 0.5, 0.55, 0.6, 0.65, 0.7, 0.75, 0.8, 0.85, 0.9, 0.95 or 1% of the RPLPO mRNA level or quantity reference or in the range of 0.2-1% of the RPLPO mRNA level or quantity reference. The level or quantity of Asf1b relative to the quantity of RPLPO mRNA may be calculated by applying x=100/(EA(Cp Gene—Cp RPLPO)), where E is the mean efficiency of the primers. For instance, with the primers set of SEQ ID Nos 5 and 6, E is 2. By “about” is intended +/−5% of the value. The exact value should be determined using a reference kit. Specifically, Asf1b mRNA levels should be determined in a number of reference cell lines with the same primers, reference gene and QPCR amplification kit that are intended to be used with the tumoral samples. For instance, see FIG. 16 clearly showing that the cut-off value may be defined with reference cell lines as a value which is above the levels of Asf1b mRNA in MCF-7 GO, BJ GO and/or Bst cell lines. A cut-off value may then be determined dependent on expression levels of Asf1b in the various cell types containing both normal and tumoral cells. This cut-off value may be easily adjusted by the skilled person using another reference gene. This cut-off value is in particular applicable for breast cancer. This value may vary depending on the type of cancer and will be easily adapted by the one skilled in the art. In particular, the skilled in the art can define the cut-off value for the expression level of Asf1b based on the study of a reference population as used in the Example section with the breast cancer samples collected in 1995 or 1996. The cut-off value will be chosen so as to obtain a significant p-value.

In a particular embodiment of these above mentioned methods, the present method further comprises assessing at least another cancer or prognosis marker such as tumor grade, hormone receptor status, mitotic index, tumor size, HJURP expression level or expression of proliferation markers such as Ki67, MCM2, CAF-1 p60 and CAF-1 p150 or prognosis marker such as HP1α, CAF-1 p60 and CAF-1 p150 (Polo et al., 2010; Masco lo et al., 2010; Staibano et al., 2011), preferably expression of CAF-1 proliferation markers and HPla prognosis marker. These markers are commonly used and the results obtained with these markers may be combined with the results obtained with the present method in order to provide a better prognostic value than the individual factor alone. This combination of chromatin-related factors to better predict prognosis is reminiscent of the combination of different chemotherapeutic agents which are used together to target a cancer with a better efficiency. The use of the proliferation and prognosis markers is well-known by the skilled person. HPla as a prognosis marker has been described in PCT application PCT/EP2010/055423 and in De Koning et al., 2009. As example, the tumor grade may be determined according the Elston & Ellis method (Elston & Ellis, 2002), the hormone receptor status (estrogen and progesterone) may be determined at the protein or at the mRNA level, the mitotic index may be determined by counting mitotic cells in ten microscopic fields of a representative tissue section and the tumor size can be detected by imaging techniques (e.g. mammography), by palpation or after surgery in the excised tissue. Expression of Ki67 and CAF-1 can be assessed at the protein level or at the mRNA level. High grade, high mitotic index, and/or large size are indicative for a worse prognosis. The HJURP expression level may be assessed as described in Hu et al., 2010. High HJURP expression is associated with poor clinical outcomes. Preferably, in order to preserve the efficiency of the method, the number of assayed markers will be as low as possible. Indeed, the efficiency of a test is the combination of its predictability and the number of assayed markers. Accordingly, the Asf1b marker is combined with a limited number of selected markers, more preferably below 5-10, still more particularly with 5, 4, 3, 2 or 1 marker(s).

For instance, expression of Ki67 may be assayed by Q-PCR with the following primers: Forward primer: ATTGAACCTGCGGAAGAGCTG (SEQ ID No 9) and Reverse primer: GGAGCGCAGGGATATTCCCTT (SEQ ID No 10). For instance, expression of HP1α may be assayed by Q-PCR with the following primer set: SEQ ID Nos 11 and 12. For instance, expression of CAF-1 p60 may be assayed by Q-PCR with the following primer set: SEQ ID Nos 17 and 18. For instance, expression of CAF-1 p150 may be assayed by Q-PCR with the following primer set: SEQ ID Nos 19 and 20. Of course, alternative primers set may be designed by the skilled in the art.

Using multivariate statistical analyses, the inventors have demonstrated, in the experimental section below, that Asf1b expression levels predict disease outcome (metastasis occurrence) better than these standard prognostic markers.

The present invention further concerns the use of Asf1b as a prognosis marker in cancer, preferably in human cancer and more preferably in human breast cancer. In a preferred embodiment, Asf1b is used as a prognosis marker in an early stage breast cancer without local or systemic invasion. As used herein, the term “prognosis marker” refers to a compound, i.e. Asf1b, used to predict or monitor clinical outcome of a subject affected with a cancer.

The present invention also concerns the use of Asf1b as a marker for selecting a subject affected with a cancer for a therapy, preferably an adjuvant therapy, or determining whether a subject affected with a cancer is susceptible to benefit from a therapy, preferably an adjuvant therapy. Preferably, the cancer is a human cancer and more preferably for human breast cancer.

The present invention further concerns the use of Asf1b as a marker for monitoring the response to a treatment of a subject affected with a cancer. Preferably, the cancer is a human cancer and more preferably for human breast cancer.

In another aspect, the present invention further concerns a kit, and its use,
(a) for predicting or monitoring clinical outcome of a subject affected with a cancer; and/or
(b) for selecting a subject affected with a cancer for a therapy, preferably an adjuvant therapy, or determining whether a subject affected with a cancer is susceptible to benefit from a therapy, preferably an adjuvant therapy; and/or
(c) for monitoring the response to a treatment of a subject affected with a cancer,
wherein the kit comprises
(i) at least one antibody specific to Asf1b and, optionally, means for detecting the formation of the complex between Asf1b and said at least one antibody; and/or
(ii) at least one probe specific to the Asf1b mRNA or cDNA and, optionally, means for detecting the hybridization of said at least one probe on Asf1b mRNA or cDNA; and/or
(iii) at least one nucleic acid primer pair specific to Asf1b mRNA or cDNA and, optionally, means for amplifying and/or detecting said mRNA or cDNA; and,
(iv) optionally, a leaflet providing guidelines to use such a kit.

In addition, the kit may further comprise the detection means specific of one or several markers selected from the group consisting of HJURP, Ki67, MCM2, HP 1a, CAF-1 p60 and CAF-1 p150. The detection means may be an antibody specific of the protein, or a probe or a primer pair specific of the mRNA or cDNA. Preferably, the Asf1b marker is combined with less than 20 other markers, preferably less than 10, 9, 8, 7, 6, 5 or 4 other markers, still more preferably less than 7, 6, 5 or 4 other markers.

The inventors have also shown that inhibition of Asf1b expression using a RNA interference approach cells prevented continued cell proliferation, thereby showing that Asf1b is a therapeutic target against which new anti-cancer drugs could be developed.

The present invention also relates to methods for selecting or identifying a molecule of interest for treating a cancer. In particular, the molecule is useful for improving the clinical outcome of a patient having a cancer. Indeed, the present invention provides a new therapeutic target, Asf1b. Therefore, any molecule able to inhibit Asf1b is of interest for treating cancer. By inhibiting Asf1b is intended that the molecule is able to inhibit the histone H3-H4 chaperone activity of Asf1b. Therefore, the present invention relates to a method for selecting or identifying a molecule of interest for treating a cancer comprising testing a molecule for its ability to inhibit Asf1b and selecting the molecule capable of inhibiting Asf1b.

In particular, the present invention further concerns methods for screening or identifying a molecule suitable for treating a cancer, in particular for improving the clinical outcome of a patient having cancer, comprising 1) providing a cell expressing the Asf1b gene; 2) contacting said cell with a test molecule; 3) determining the expression level of said Asf1b gene or the activity of the Asf1b protein in said cell; and, 4) selecting the molecule which decreases the expression level or activity of said Asf1b gene or protein, respectively. In a particular embodiment, the cell-expression the Asf1b gene is obtained from a patient sample. Preferably, the cell is a mammalian cell expressing the Asf1b gene. More preferably, the cell has a high expression level of Asf1b. The expression level of said Asf1b gene may be determined by measuring the quantity of Asf1b protein or Asf1b mRNA, in particular as detailed above. The “high expression level” is determined according to the criteria detailed above. Alternatively, the activity of Asf1b protein or the expression level of Asf1b gene can be measured directly or indirectly. For instance, a decreased expression of Asf1b leads to an altered nuclear morphology and micronuclei formation (see Example section and FIGS. 7D-E). For instance, nuclei can be observed by microscopy with DAPI or other revelation systems. Therefore, the Asf1b activity may be measured by observing nuclear morphology and an altered nuclear morphology is indicative of a decreased Asf1b activity or Asf1b inhibition. In addition or alternatively, the activity of Asf1b protein or the expression level of Asf1b gene can be measured by a Colony Formation Assay. Indeed, a decreased expression of Asf1b leads to a decreased number of colonies (see Example section and FIG. 7C). A Colony Formation Assay can be carried out as described in Franken et al., 2006. In a preferred embodiment, the activity of Asf1b protein or the expression level of Asf1b gene is measured both by the nuclear morphology and by the Colony Formation Assay.

Alternatively, the present invention further concerns in vitro methods for screening or identifying a molecule suitable for treating a cancer, in particular for improving the clinical outcome of a patient having cancer, comprising 1) contacting a test molecule with Asf1b protein, 2) determining the ability of the test molecule to bind said Asf1b protein, and 3) selecting the test molecule capable of binding said Asf1b protein. Binding to said Asf1b protein provides an indication as to the ability of the molecule to inhibit or decrease the activity of said Asf1b protein. The determination of binding may be performed by various techniques, such as by labeling of the test molecule, by competition with a labeled reference ligand, etc. The binding capacity or affinity to Asf1b can be measured by well-known technologies, such as Biacore. In particular, the binding assay can be combined with functional assay such as the nuclear morphology determination or the Colony Formation Assay.

The present invention also concerns methods for screening or identifying a molecule suitable for treating a cancer, in particular for improving the clinical outcome of a patient having cancer, based on an in vitro nucleosome assembly assay. This assay is based on the measurement of the ability of the test molecule to interfere with nucleosome assembly, in the replication-coupled or the replication-independent pathway or both pathways. Details on in vitro nucleosome assembly assays can be found for instance in Ray-Gallet et al., 2004 (in particular pages 123-127).

Accordingly, in the screening methods of the invention, the ability of the test molecule to inhibit Asf1b is measured by a binding assay to Asf1b protein, by an assay measuring the Asf1b expression in a cell, by a nuclear morphology assay, by a Colony Formation Assay, by an in vitro nucleosome assembly assay or by a combination thereof (combination of two, three or four assays).

The test molecule can be any organic or inorganic molecule and in particular, without being limiting thereto, a small molecule, an aptamer, a lipid, an antibody, a nucleic acid or a peptide, polypeptide or protein. The molecule may be all or part of a combinatorial library of products, for instance.

Finally, the invention relates to a method for treating a cancer by administering a therapeutic effective amount of a molecule inhibiting the Asf1b or the use of a molecule inhibiting the Asf1b for treating a cancer. In particular, the treatment allows the improvement of the clinical outcome of a patient having cancer.

Accordingly, the present invention relates to
a pharmaceutical composition comprising a molecule inhibiting Asf1b, and optionally a pharmaceutically acceptable carrier, in particular for use in the treatment of cancer, optionally in combination with radiotherapy or an anti-tumoral agent;
a molecule inhibiting Asf1b, and optionally a pharmaceutically acceptable carrier, for use in the treatment of cancer, optionally in combination with radiotherapy or an anti-tumoral agent;
the use of a molecule inhibiting Asf1b for the manufacture of a medicament for the treatment of cancer, optionally in combination with radiotherapy or an anti-tumoral agent;
a method for treating a cancer in a subject in need thereof, comprising administering an effective amount of a pharmaceutical composition comprising a molecule inhibiting Asf1b and optionally a pharmaceutically acceptable carrier;
a combined preparation, product or kit containing (a) a molecule inhibiting Asf1b and (b) an anti-tumoral agent as a combined preparation for simultaneous, separate or sequential use, in particular in the treatment of cancer;
a method for treating a cancer in a subject in need thereof, comprising administering an effective amount of a pharmaceutical composition comprising a molecule inhibiting Asf1b, and an effective amount of a pharmaceutical composition comprising an anti-tumoral agent; and,
a method for treating a cancer in a subject in need thereof, comprising administering an effective amount of a pharmaceutical composition comprising a molecule inhibiting Asf1b in combination with radiotherapy;

Within the context of the invention, the term treatment denotes curative, symptomatic, and preventive treatment. Pharmaceutical compositions and preparations of the invention can be used in humans with existing cancer or tumor, including at early or late stages of progression of the cancer. The pharmaceutical compositions and preparations of the invention will not necessarily cure the patient who has the cancer but will delay or slow the progression or prevent further progression of the disease, ameliorating thereby the patients' condition. In particular, the pharmaceutical compositions and preparations of the invention reduce the development of tumors, reduce tumor burden, produce tumor regression in a mammalian host and/or prevent metastasis occurrence and cancer relapse. In treating the cancer, the pharmaceutical composition of the invention is administered in a therapeutically effective amount.

By “effective amount” it is meant the quantity of the pharmaceutical composition of the invention which prevents, removes or reduces the deleterious effects of cancer in mammals, including humans. It is understood that the administered dose may be adapted by those skilled in the art according to the patient, the pathology, the mode of administration, etc.

The cancer may be a solid cancer or a hematopoietic cancer, preferably a solid cancer and more preferably, an early stage solid cancer without local or systemic invasion. Preferably, the cancer is selected from the group consisting of breast cancer, osteosarcoma, skin cancer, ovarian cancer, lung cancer, liver cancer, cervix cancer, liposarcoma, gastric cancer, pancreatic cancer, bladder cancer, vulvar cancer, colon cancer and brain cancer. More preferably, the cancer is breast cancer. Even more preferably, the cancer is an early stage breast cancer without local or systemic invasion.

In a particular embodiment, the cancer to be treated is a cancer with a high expression level of Asf1b. A high expression of Asf1b can be assayed as above-detailed.

In a preferred embodiment, the molecule inhibiting Asf1b is selective in respect to Asf1a. That means that the molecule inhibits Asf1b and not Asf1a, or with a greater efficacy for Asf1b in comparison to Asf1a (for instance by a factor of at least 10, 100 or 1000). However, due to the differential expression/activity of the iso forms Asf1b and Asf1a, a non-selective inhibitor of Asf1 can also be useful especially in cases where such inhibitor could be selectively targeted to proliferating cancer cells.

The Asf1b inhibitor can be, without being limiting thereto, a small molecule, an aptamer, an antibody, a nucleic acid or a molecule preventing the interaction Asf1b with an Asf1b interacting partner. Preferably, the molecule inhibiting Asf1b is selected from the group consisting of an antibody against Asf1b, an antisense or siRNA against Asf1b, preferably the antibody, antisense or siRNA being specific of Asf1b in comparison to Asf1a.

The term “small molecule” refers to a molecule of less than 1,000 daltons, in particular organic or inorganic compounds. Structural design in chemistry should help to find such a molecule. The molecule may have been identified by a screening method disclosed in the present invention.

In a preferred embodiment of the invention, the Asf1b inhibitor is a nucleic acid molecule interfering specifically with Asf1b expression, thereby decreasing or suppressing the expression of this protein. Such nucleic acids are more amply detailed below. Preferably, this nucleic acid is selected from the group consisting of a RNAi, an antisense nucleic acid or a ribozyme. Said nucleic acid can have a sequence from 15 to 50 nucleotides, preferably from 15 to 30 nucleotides. In a preferred embodiment, said nucleic acid comprises a sequence of SEQ ID No 14. By a “decrease” in expression is meant, for example, a decrease of 30%, 50%, 70%, 80%, 90% or 95% of the gene expression product.

The term “RNAi” or “interfering RNA” means any RNA which is capable of down-regulating the expression of the targeted protein. It encompasses small interfering RNA (siRNA), double-stranded RNA (dsRNA), single-stranded RNA (ssRNA), micro-RNA (miRNA), and short hairpin RNA (shRNA) molecules. RNA interference, designate a phenomenon by which dsRNA specifically suppresses expression of a target gene at post-translational level. In normal conditions, RNA interference is initiated by double-stranded RNA molecules (dsRNA) of several thousands of base pair length. In vivo, dsRNA introduced into a cell is cleaved into a mixture of short dsRNA molecules called siRNA. The enzyme that catalyzes the cleavage, Dicer, is an endo-RNase that contains RNase III domains (Bernstein et al., 2001). In mammalian cells, the siRNAs produced by Dicer are 21-23 by in length, with a 19 or 20 nucleotides duplex sequence, two-nucleotide 3′ overhangs and 5′-triphosphate extremities (Elbashir et al., 2001a; Elbashir et al., 2001b; Zamore et al., 2000). A number of patents and patent applications have described, in general terms, the use of siRNA molecules to inhibit gene expression, for example, WO 99/32619, US 20040053876, US 20040102408 and WO 2004/007718.

siRNA are usually designed against a region 50-100 nucleotides downstream the translation initiator codon, whereas 5′UTR (untranslated region) and 3′UTR are usually avoided. The chosen siRNA target sequence should be subjected to a BLAST search against EST database to ensure that the only desired gene is targeted. Various products are commercially available to aid in the preparation and use of siRNA. In a preferred embodiment, the RNAi molecule is a siRNA of at least about 15-50 nucleotides in length, preferably about 20-30 base nucleotides.

RNAi can comprise naturally occurring RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end of the molecule or to one or more internal nucleotides of the RNAi, including modifications that make the RNAi resistant to nuclease digestion.

RNAi may be administered in free (naked) form or by the use of delivery systems that enhance stability and/or targeting, e.g., liposomes, or incorporated into other vehicles, such as hydrogels, cyclodextrins, biodegradable nanocapsules, bioadhesive microspheres, or proteinaceous vectors (WO 00/53722), or in combination with a cationic peptide (US 2007275923). They may also be administered in the form of their precursors or encoding DNAs.

Antisense nucleic acid can also be used to down-regulate the expression of Asf1b. The antisense nucleic acid can be complementary to all or part of a sense nucleic acid encoding Asf1b e.g., complementary to the coding strand of a double-stranded cDNA molecule or complementary to an mRNA sequence, and is thought to interfere with the translation of the target mRNA. Preferably, the antisense nucleic acid is an RNA molecule complementary to a target mRNA encoding Asf1b.

An antisense nucleic acid can be, for example, about 5, 10, 15, 20, 25, 30, 35, 40, 45 or 50 nucleotides in length. Particularly, antisense RNA molecules are usually 18-50 nucleotides in length.

An antisense nucleic acid can be constructed using chemical synthesis and enzymatic ligation reactions using procedures known in the art. Particularly, antisense RNA can be chemically synthesized, produced by in vitro transcription from linear (e.g. PCR products) or circular templates (e.g., viral or non-viral vectors), or produced by in vivo transcription from viral or non-viral vectors.

Antisense nucleic acid may be modified to have enhanced stability, nuclease resistance, target specificity and improved pharmacological properties. For example, antisense nucleic acid may include modified nucleotides designed to increase the physical stability of the duplex formed between the antisense and sense nucleic acids, e.g., phosphorothioate derivatives and acridine substituted nucleotides.

Ribozyme molecules can also be used to block the expression of Asf1b. Ribozymes are catalytic RNA molecules with ribonuclease activity which are capable of cleaving a single-stranded nucleic acid, such as an mRNA, to which they have a complementary region. Thus, ribozymes can be used to catalytically cleave mRNA transcripts to thereby inhibit translation of the protein encoded by the mRNA. Ribozyme molecules specific for Asf1b can be designed, produced, and administered by methods commonly known to the art (see e.g., Fanning and Symonds, 2006, reviewing therapeutic use of hammerhead ribozymes and small hairpin RNA).

In a particular embodiment, the interfering nucleic acid molecule is expressed by a vector, preferably a viral vector comprising a contruct allowing the expression of interfering nucleic acid molecule. For instance, the viral vector can be an adenovirus, an adeno-associated virus, a lentivirus or a herpes simplex virus.

In another preferred embodiment of the invention, the Asf1b inhibitor is an antibody specific of Asf1b and able to decrease its activity. Preferably, the antibody is specific of Asf1b, and capable of discriminating binding to Asf1b in comparison with Asf1a.

As used in the present invention, the term “antibody” includes monoclonal antibodies, chimeric antibodies, humanized antibodies, recombinant antibodies and fragments thereof. Antibody fragment means, for example, F(ab)2, Fab, Fab′ or sFv fragments. According to a particular embodiment, the antibody can be IgG, IgM, IgA, IgD or IgE, preferably IgG or IgM. Methods for producing antibodies are well known to those persons skilled in the art.

Antibodies specific of Asf1b have been disclosed in details above. However, in order to be effective, such antibodies might have to be engineered to be able to penetrate the cellular membrane into the nucleus.

As used here, the term “aptamer” means a molecule of nucleic acid or a peptide able to bind specifically to Asf1b protein or to a binding partner of Asf1b. In a preferred embodiment, the aptamers are nucleic acids, preferably RNA, generally comprising between 5 and 120 nucleotides (Osborne et al., 1997). They can be selected in vitro according to a process known as SELEX (Systematic Evolution of Ligands by Exponential Enrichment).

The molecule preventing interaction of Asf1b with one or several of its binding partners can be an aptamer, an antibody, a peptide, a polypeptide or a protein. Preferably, the binding partner for which preventing the interaction with Asf1b is sought is selected from the group consisting of Histone H3, Histone H4, CAF-1, or HIRA.

In particular, the structure of Asf1 complexed with histones H3 and H4 is known (English et al, 2006; Natsume et al, 2007). Accordingly, based on this knowledge, the skilled in the art can design molecules preventing the binding of Asf1b to histone H3 and/or H4. For instance, a segment of Asf1b or of its binding partner can be prepared and used to prevent the binding.

In addition, the structure of Asf1 complexed with a specific domain of HIRA or CAF-1 p60 called the B-domain is known (Tang et al., 2006; Malay et al., 2008). Accordingly, based on this knowledge, the skilled in the art can design molecules preventing the binding of Asf1b to p60 or HIRA. For instance, a segment of Asf1b or of its binding partner can be prepared and used to prevent the binding. In particular, a fraction of the B-domain of HIRA represents an attractive candidate for an inhibitory molecule and can act as a dominant negative as illustrated in the experimental section. For example, the B-domain may comprise or consist of the sequence of SEQ ID No. 28. Interestingly, as interaction with the histones H3-H4 occurs on the opposite site, an inhibiting molecule could be designed covering both H3-H4 and HIRA/CAF-1 p60 interaction sites.

Furthermore, the inventors have herein identified novel structural differences between Asf1a and Asf1b. They have defined two regions mediating the specificity of the Asf1a and Asf1b isoforms, these regions being distinct from the previously characterized interaction domains of Asf1 with histones and with HIRA or CAF-1 p60 (FIG. 21B). They have also demonstrated the preferential interaction of Asf1b with CAF-1 p60 (FIG. 21C). Accordingly, based on this knowledge, the skilled person can easily design molecules, in particular peptides, that specifically prevent the binding of Asf1b with HIRA or CAF-1 p60, more particularly with CAF-1 p60.

The molecule inhibiting Asf1b may be administered by topical, transdermal, oral, rectal, sublingual, intranasal or parenteral route.

Further aspects and advantages of the present invention will be described in the following examples, which should be regarded as illustrative and not limiting.

### EXAMPLES

### Example 1

Here, the inventors investigated the specific expression of the two human Asf1 isoforms, Asf1a and Asf1b, in relation to cell proliferation and tumorigenesis. In model cell lines, they showed that Asf1b displays a specific proliferation-dependent expression pattern not shared by Asf1a. The specific depletion of each isoform by siRNA enabled them to evaluate their respective functional importance. They revealed a distinct genome-wide specificity for the two Asf1 iso forms by a transcriptional signature in human cells depleted of Asf1a, Asf1b or both isoforms. In addition, depletion of Asf1b led to an increased number of aberrant nuclear structures and micronuclei and severely impaired proliferation, defects that they did not observe upon Asf1a depletion.

As a further means to explore the physiological relevance of these differences, they pursued an analysis on a selection of breast tumor samples for which a long-term patient follow-up was available. Remarkably, high Asf1b expression significantly correlates with the tumor proliferation status, the appearance of metastasis and a decreased overall survival of the patients.

The inventors thus reveal here distinct functions for each Asf1 isoform with a key role for Asf1b in proliferation. Asf1b thus represents a new proliferation marker, which is relevant both for the diagnosis and the prognosis in breast cancer and potentially a new target for drug discovery in breast cancer.

### Results

### Asf1a and Asf1b Levels in Proliferating and Non Proliferating Cells

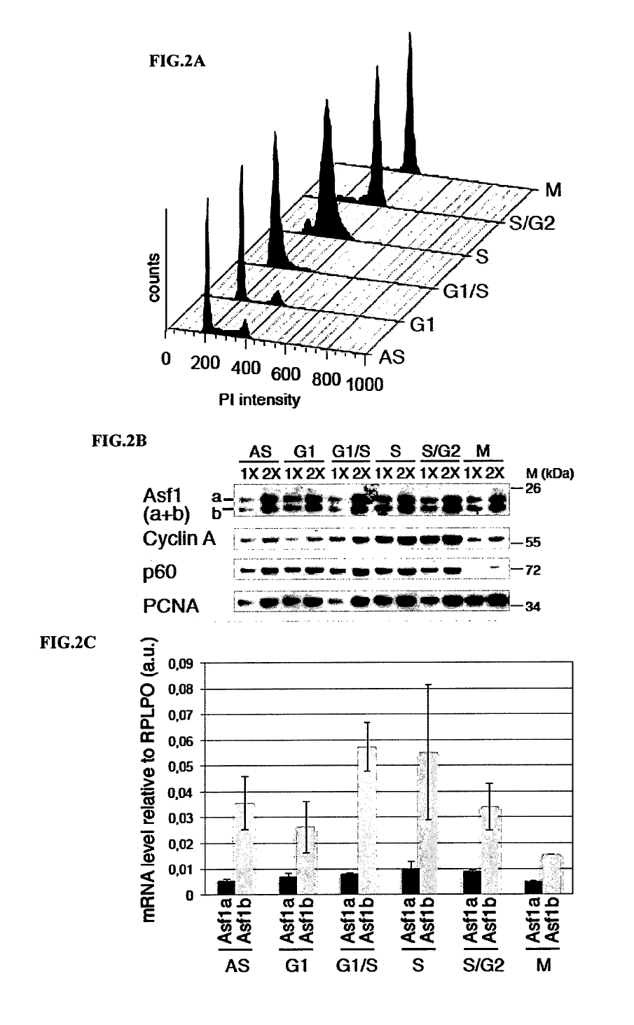

To examine the expression of Asf1 isoforms in distinct proliferating states, the inventors used specific antibodies that they developed against full-length Asf1a or the C-terminal region of Asf1b, and which recognize only one or the other isoform (FIG. 1A-D). Importantly, while their distinct migration on SDS-PAGE enables to distinguish Asf1a and Asf1b, for immunofluorescence studies the use of these antibodies was essential to specifically assess the presence of Asf1a or Asf1b in single cells. The inventors first analyzed the expression of Asf1a and Asf1b during the cell cycle in synchronized HeLa cells (FIG. 2A). Western blot analysis revealed that Asf1a and b are expressed at comparable levels during all stages of the cell cycle as previously reported (FIG. 2B). In addition, the inventors only detected minor variations for Asf1a and Asf1b at the RNA level as analyzed by quantitative RT-PCR during the cell cycle (FIG. 2C). This showed that Asf1a and Asf1b expression are not restricted to S phase but may have other functions besides their known activity during DNA replication.

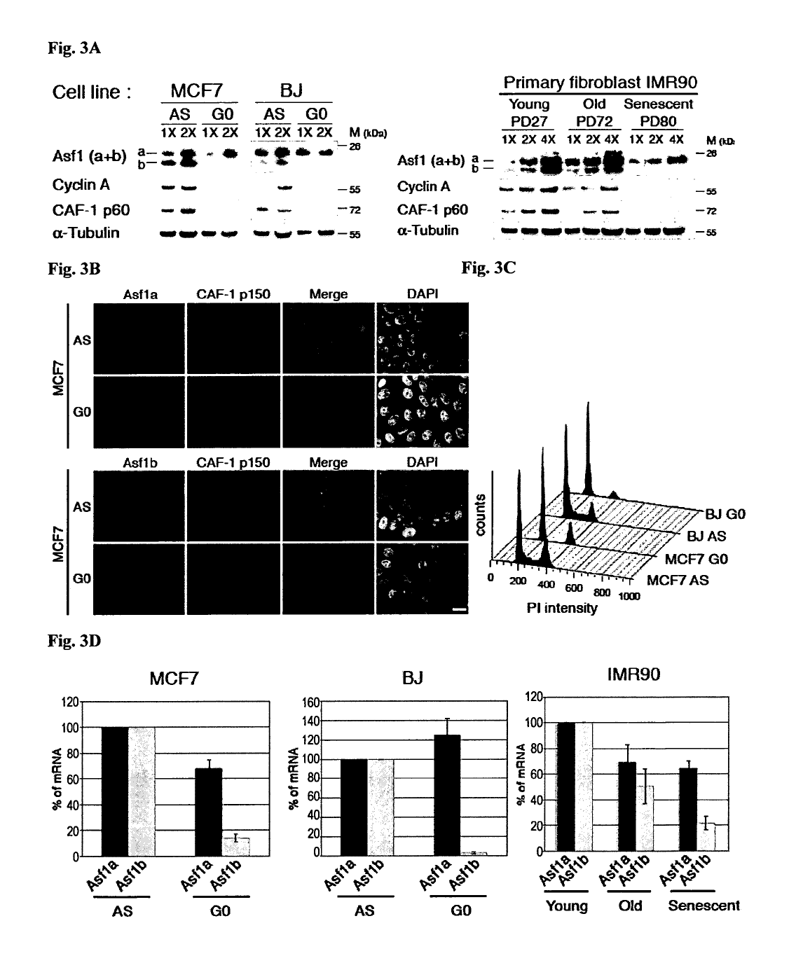

Since, the inventors did not observe significant variation during the cell cycle and given the differential expression of Asf1 isoforms in human tissues, they investigated the levels of Asf1 isoforms in non-cycling cells. To address this issue, they used various cell lines in which a transient cell cycle exit can be induced in a controlled manner. An anti-estrogen treatment puts MCF7 breast carcinoma cells in quiescence (Carroll et al., 2000), and under these conditions the inventors observed a dramatic decrease in the levels of Asf1b protein (FIG. 3A). Quantification by chemiluminescence revealed a major downregulation of Asf1b of at least 5-fold in quiescent cells. In contrast, the inventors only observed a minor decrease of Asf1a. Cyclin A and the histone chaperone CAF-1 p60 served as cell cycle controls (Polo et al., 2004). The inventors further confirmed this distinct expression of Asf1 iso forms in relation to proliferation by immunofluorescence microscopy using our specific antibodies (FIG. 3B).

The inventors then also examined the levels of Asf1a and Asf1b mRNA in asynchronous or quiescent cells by quantitative RT-PCR. While Asf1a mRNA levels remained stable upon quiescent induction, they observed a significant reduction of Asf1b mRNA levels (about 7-fold) in quiescence. These findings indicate that the most important regulatory impact on Asf1b expression occurs at the RNA level (transcription activity and/or RNA stability) (FIG. 3D). Serum starvation induced quiescence in a human primary fibroblast cell line (BJ), as attested by flow cytometry (FIG. 3C), provided similar results (FIGS. 3A-D). Further, as a means to monitor the expression of Asf1 isoforms upon cell cycle exit, the inventors choose replicative senescence, a permanent cell cycle arrest, which occurs through replication-dependent telomere shortening in human primary cells after prolonged division in culture. This was important given the role of senescence in tumor suppression and its connection with the ageing process. They found that Asf1b isoform, both at the protein and mRNA levels in young (PD30), old (PD72) and senescent (PD80) IMR90 human diploid primary fibroblasts, follows the replication capacity of the cells showing a major downregulation in senescent cells (about 5-fold) while Asf1a expression was only mildly affected (FIGS. 3A and D).

They conclude that Asf1b, in contrast to Asf1a, is a specific marker for discriminating between cycling and non-cycling cells, whether transiently or permanently arrested.

### Expression Pattern of Asf1 Isoforms Upon Cell Cycle Entry

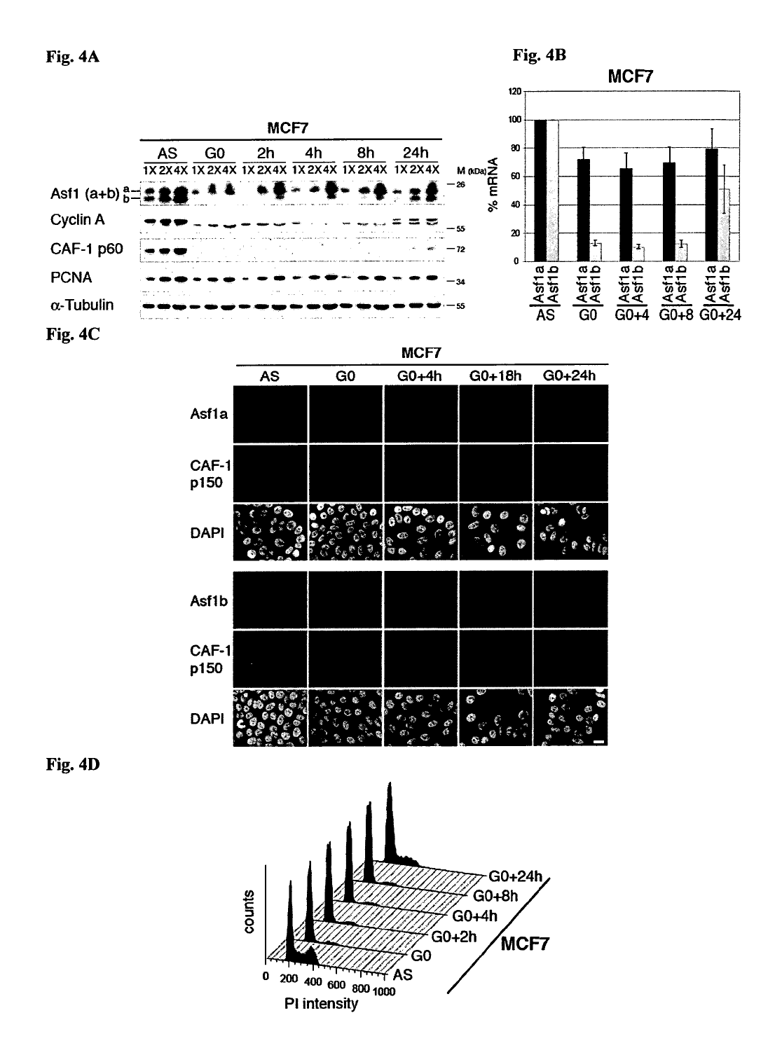

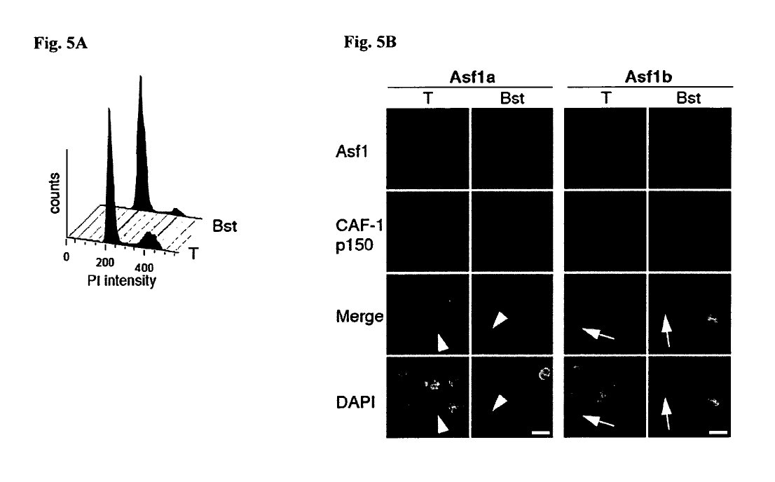

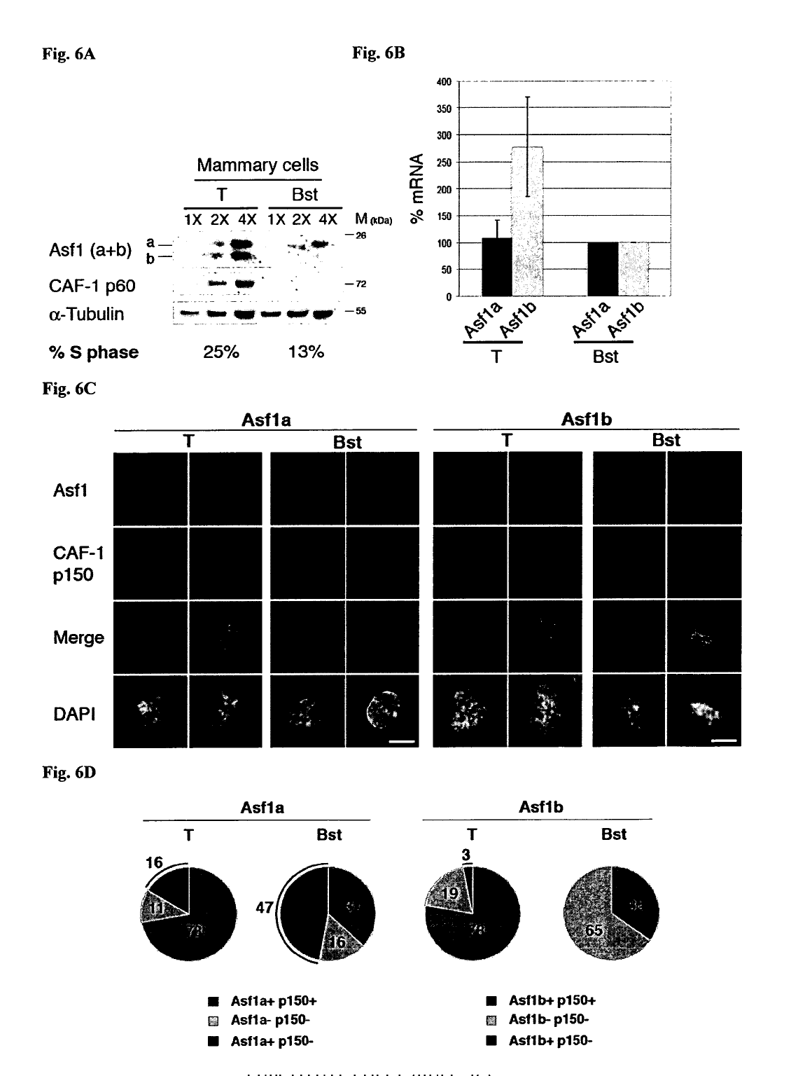

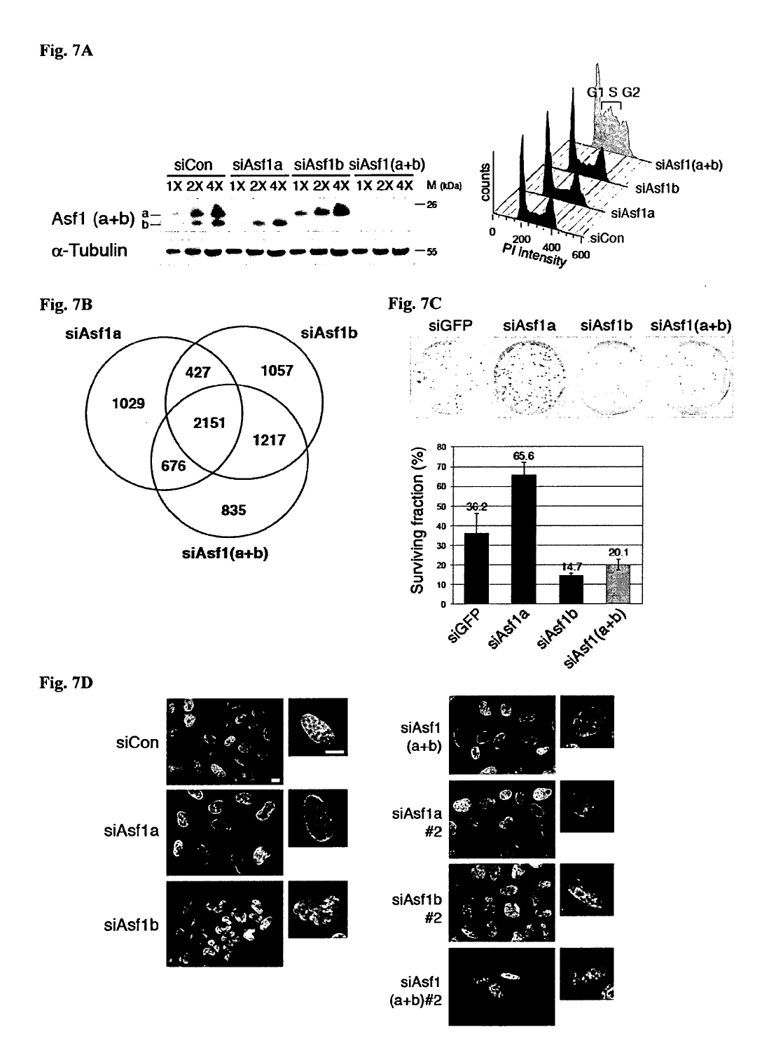

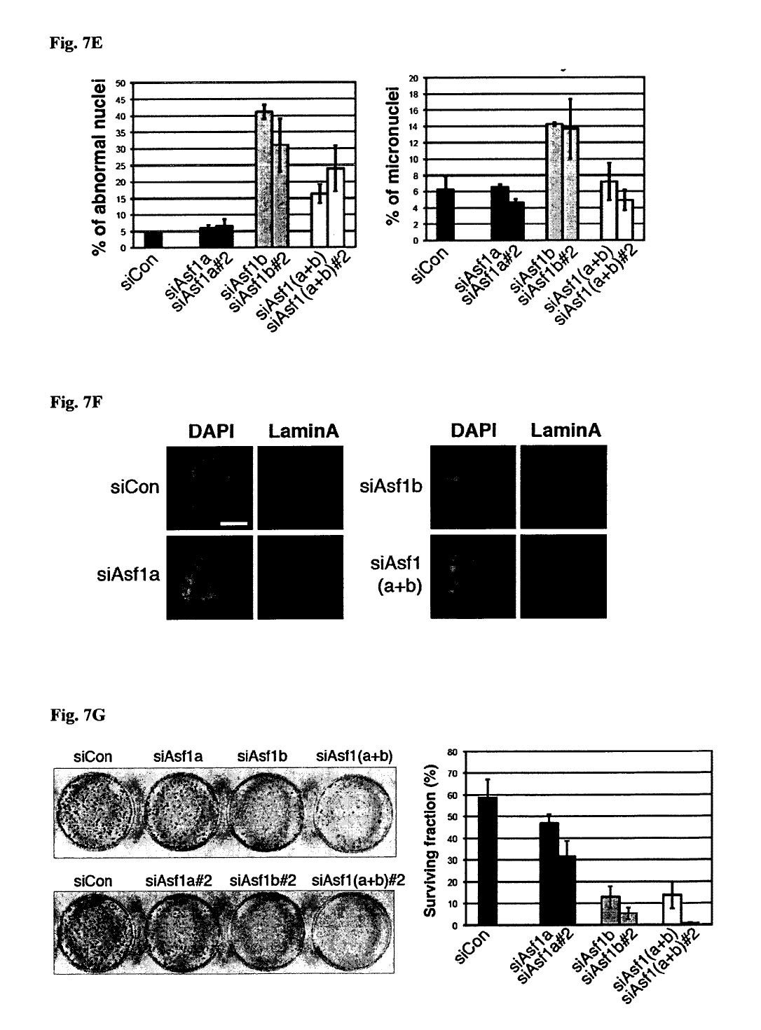

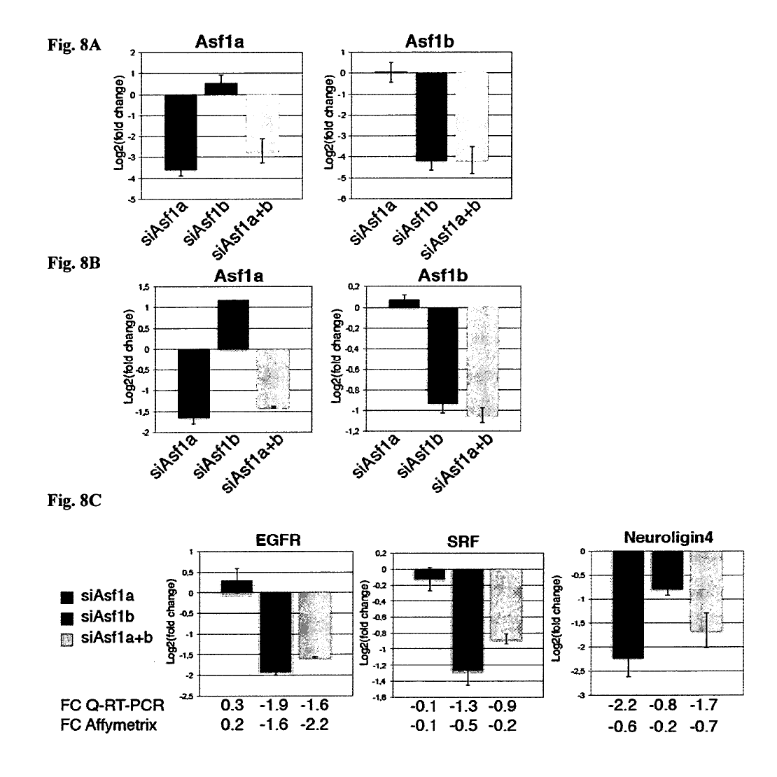

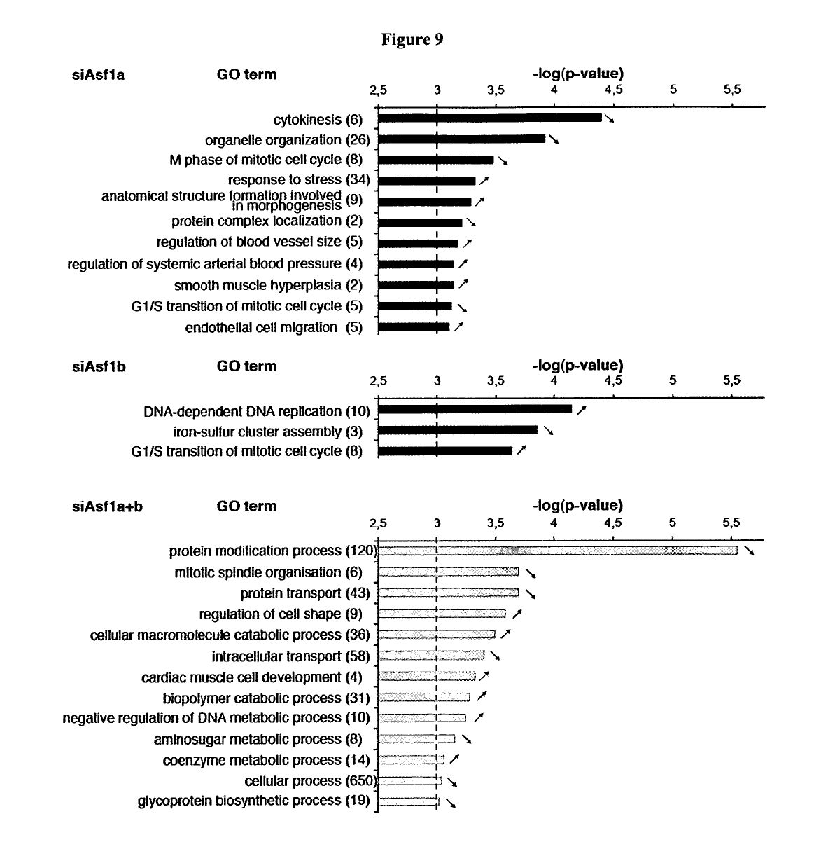

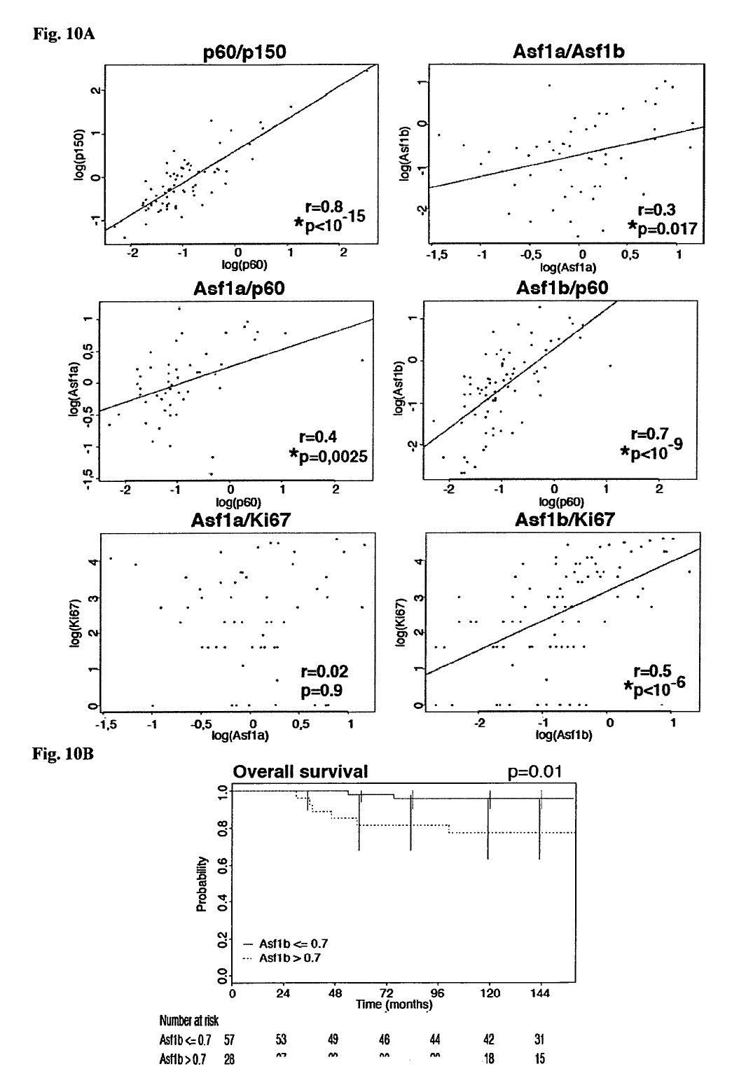

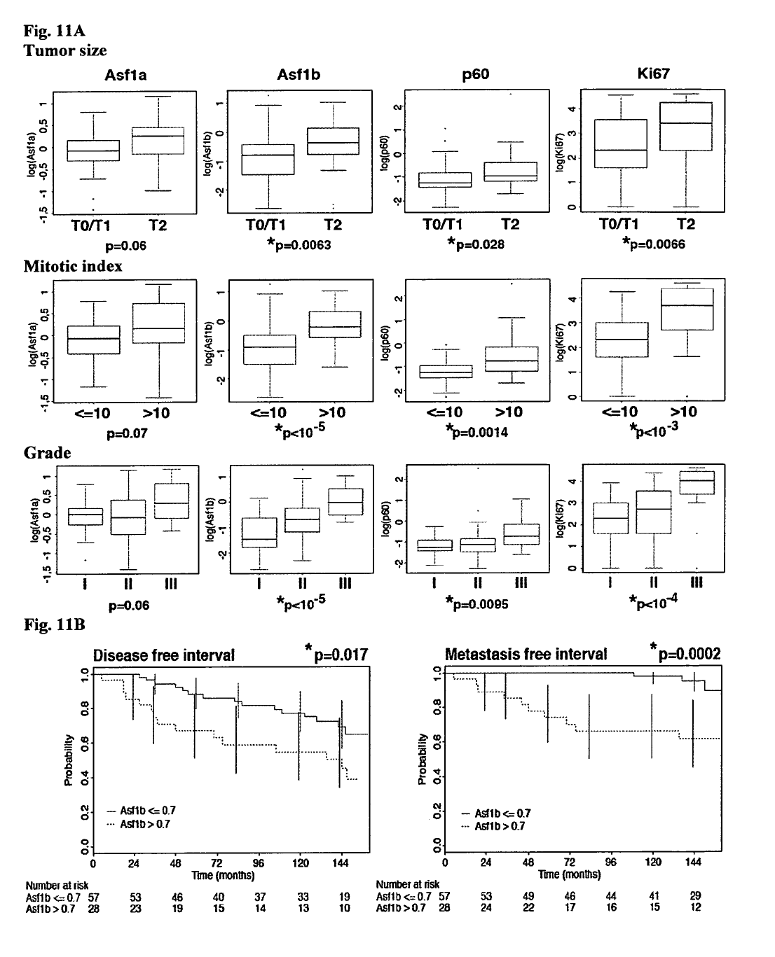

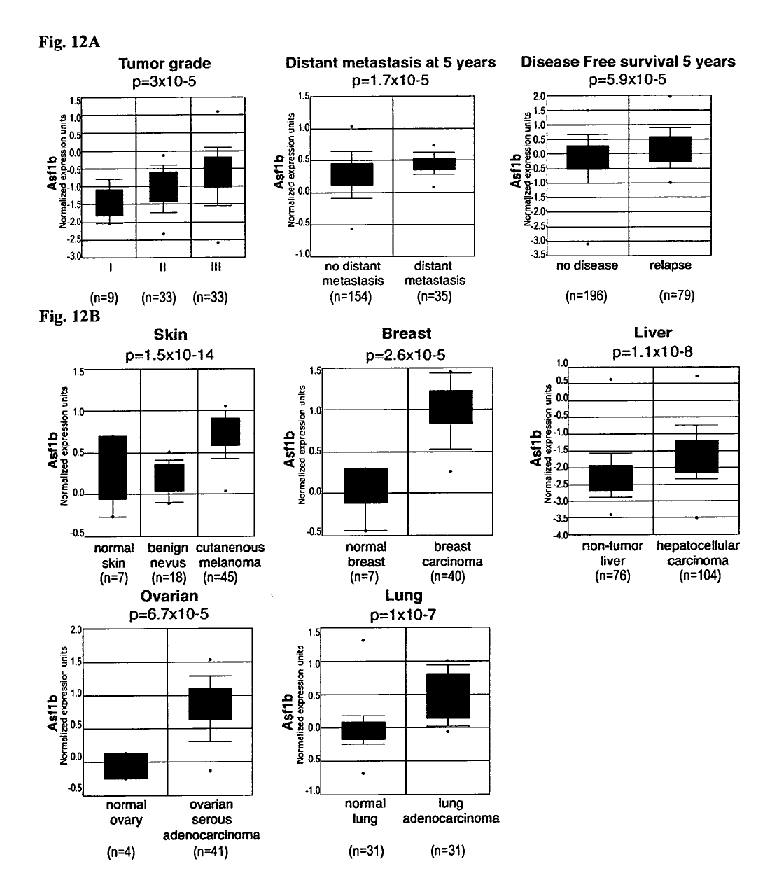

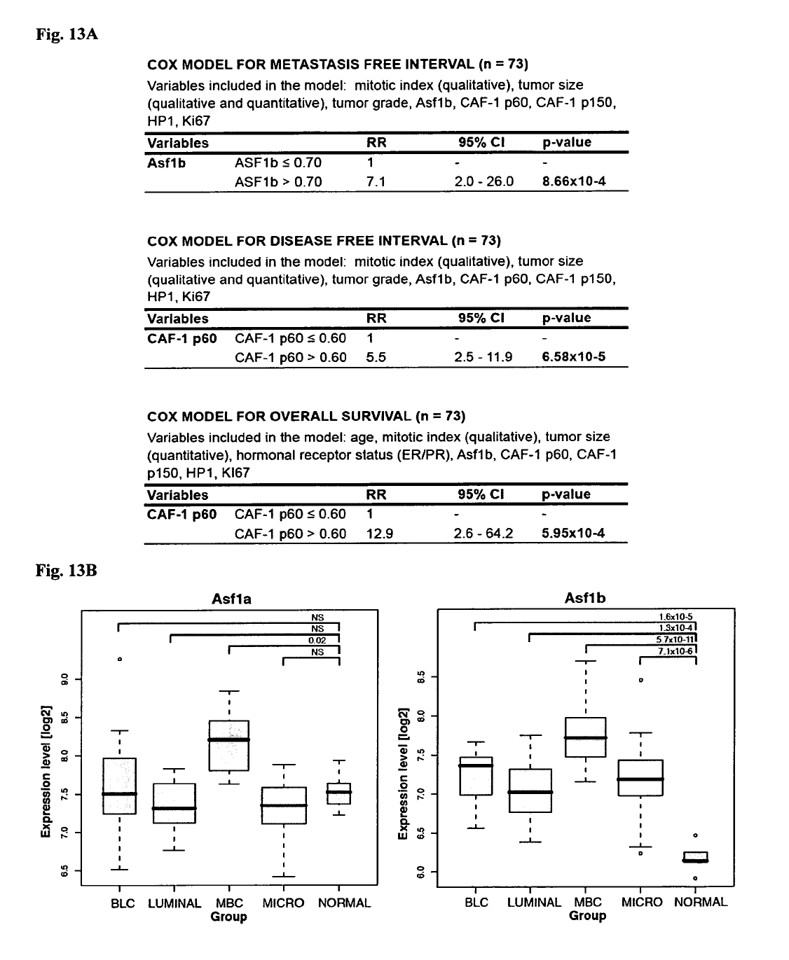

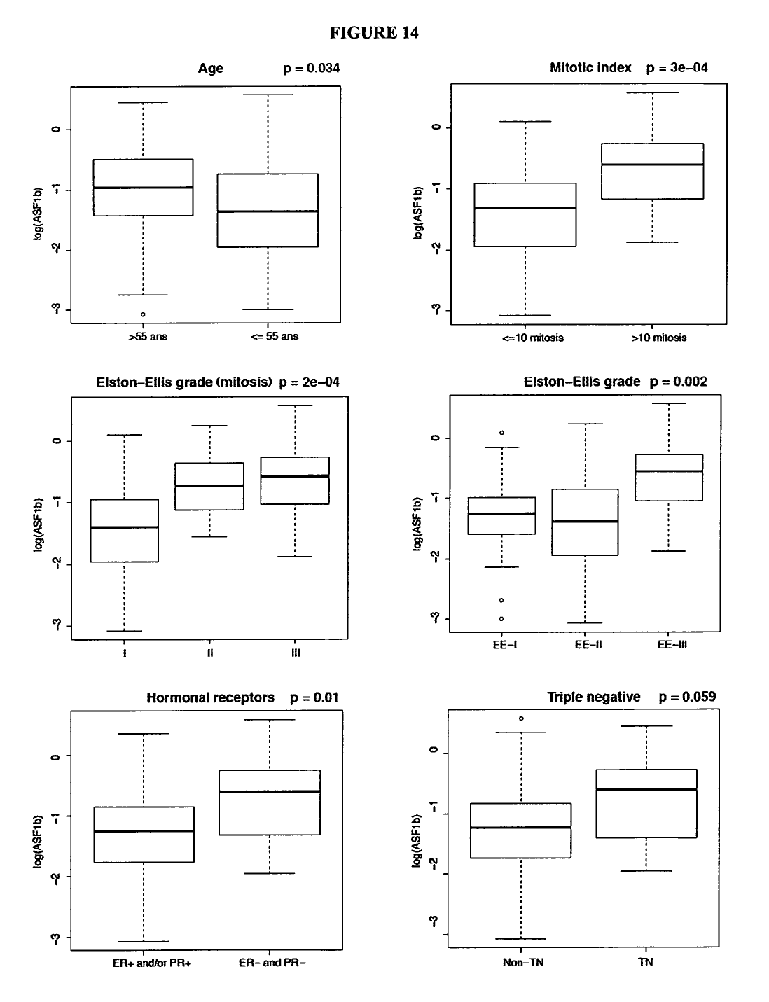

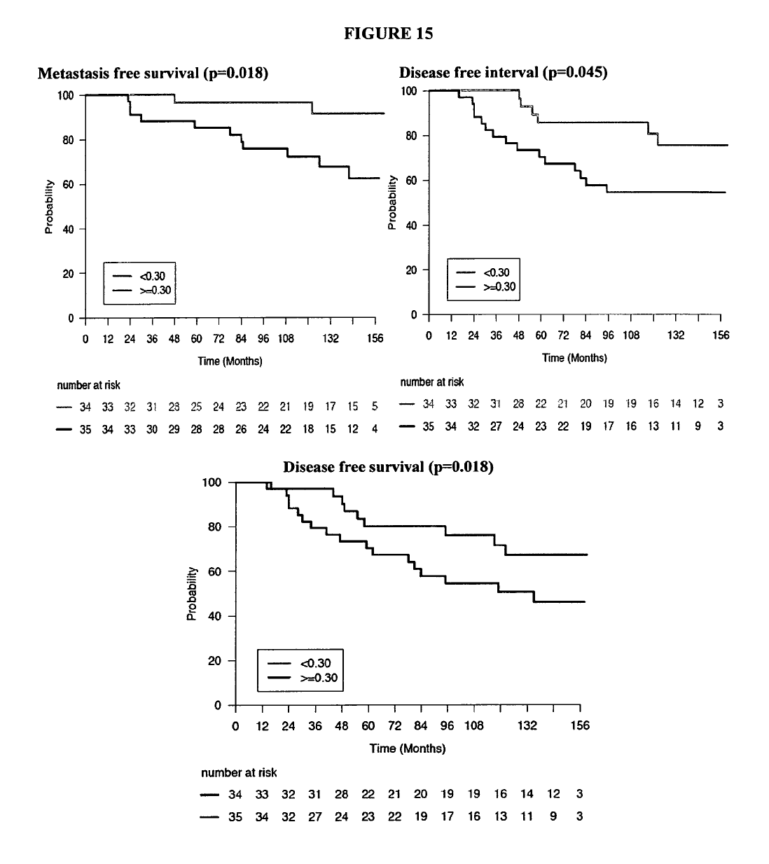

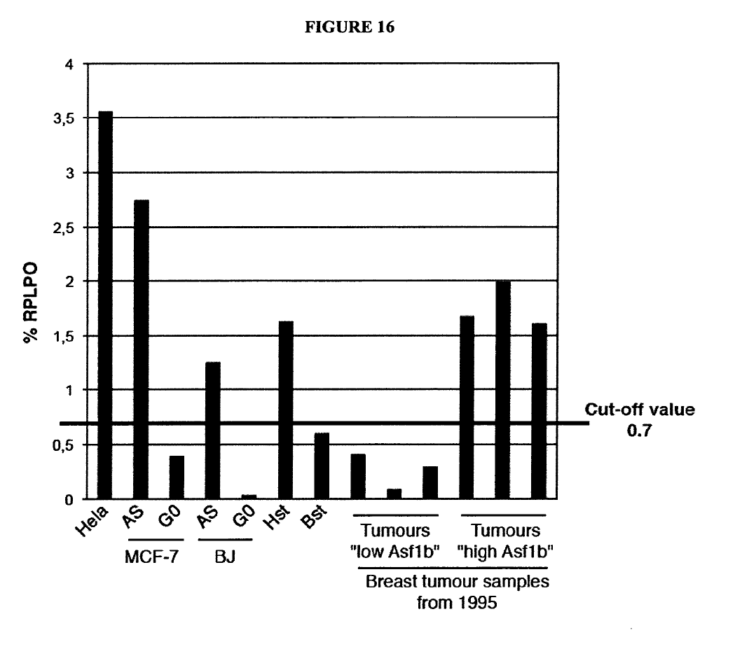

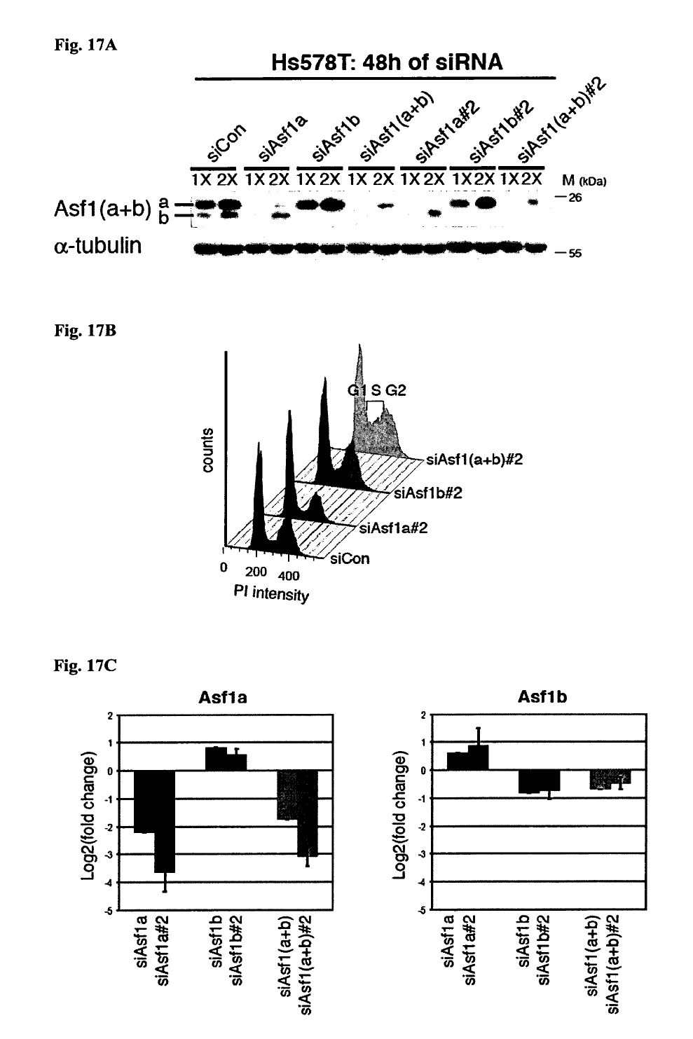

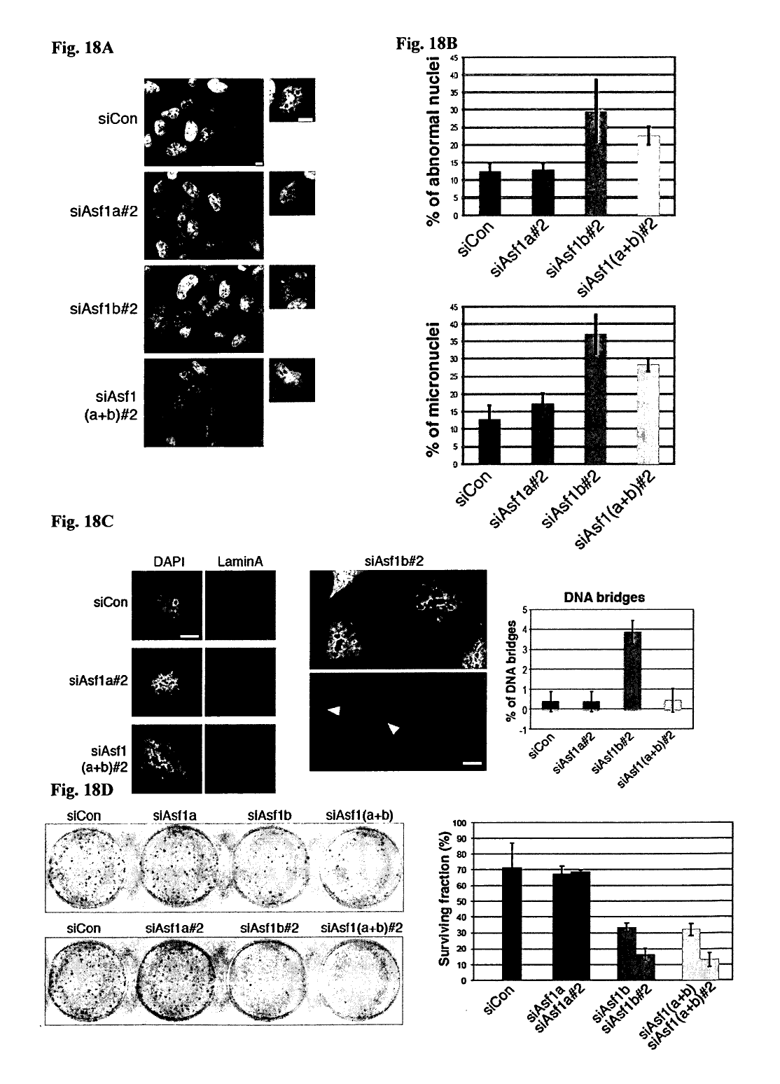

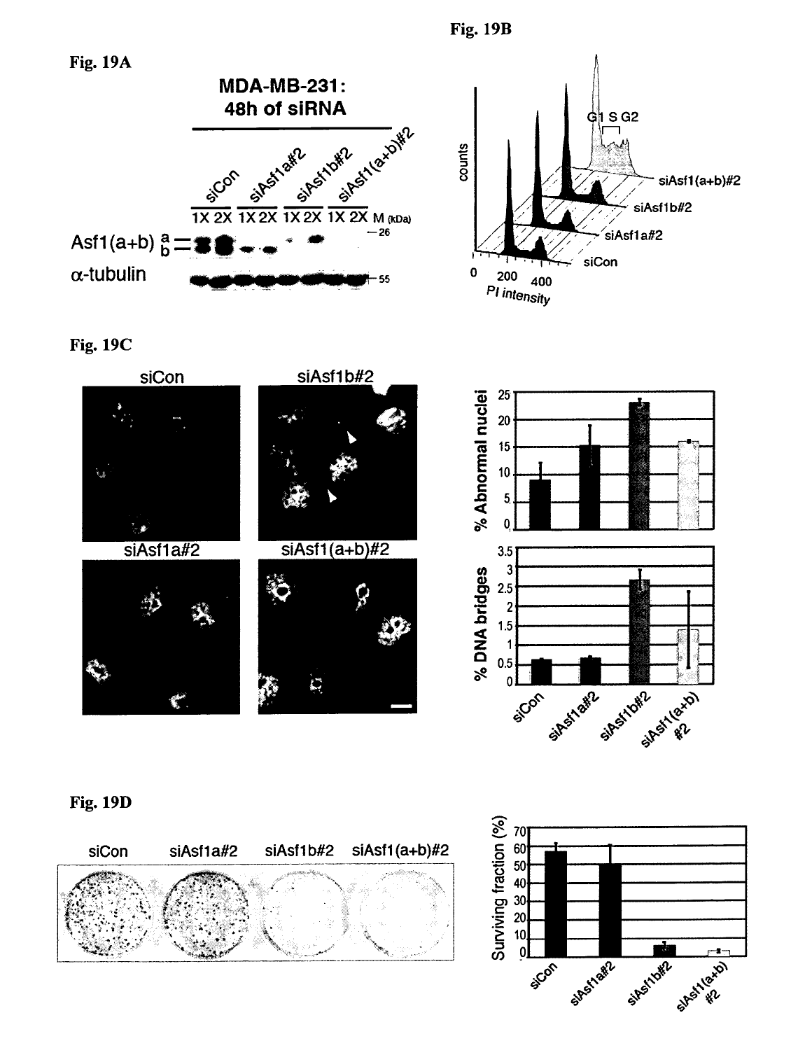

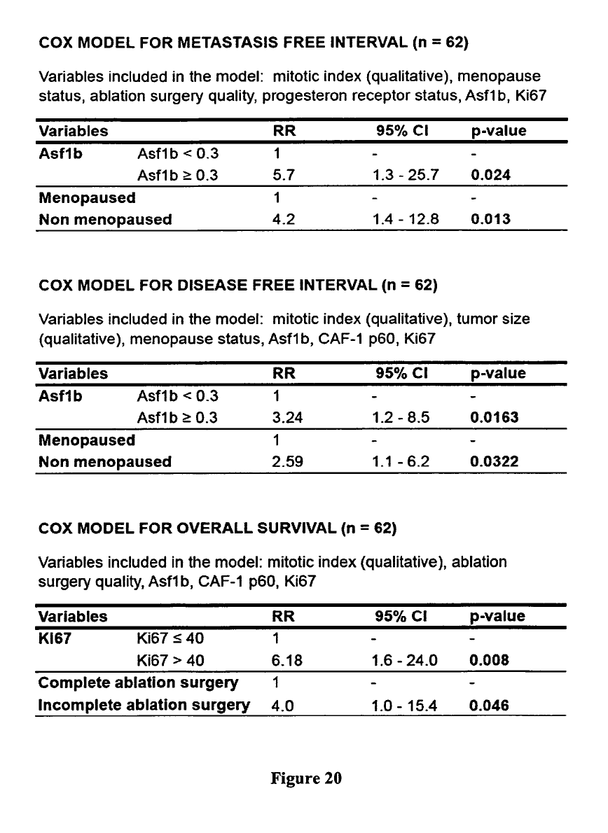

The inventors' observation of a major downregulation of Asf1b in quiescence (G0), prompted them to follow how and when Asf1b is re-expressed upon release from G0 in MCF7 cells. They monitored cell cycle progression both by flow cytometry and by the expression of the known cell cycle regulator Cyclin A, or the proliferation markers CAF-1 p60 or p150 (FIGS. 4A, C and D) (Polo et al., 2004). Western blotting revealed that, during release from GO, re-expression of Asf1b correlated with the reappearance of Cyclin A (FIG. 4A). Remarkably, the changes observed at the protein level paralleled those observed at the mRNA level as measured by quantitative RT-PCR (FIG. 4B). The inventors obtained similar results in BJ primary foreskin cells released from G0. By microscopy, they also found that the number of cells positive for Asf1b increased after release from quiescence and corresponded to cells expressing CAF-1 p150 (FIG. 4C). The rapid upregulation of Asf1b following exit from G0 and entry into the cell cycle is consistent with important cellular demands at early steps prior to S phase. Together, these data demonstrate that Asf1b is expressed in a manner dependent on the cycling status.

Asf1b Correlates with the Proliferation Status of Breast Cancer Cell Lines

Since tumoral cells often show a high proliferation rate, the inventors wondered whether Asf1 isoforms are differentially expressed in human tumoral mammary cell lines, relative to normal cells. To examine this issue, they used mammary cells derived from the same patient: Hs578T tumoral and Hs578Bst normal cells which provide a direct comparison between cells of similar origin (Hackett et al., 1977). Hs578T tumoral and Hs578Bst normal cells contain 25 and 13% of cells in S phase respectively (FIG. 5A). Western blot analysis of total cell extracts revealed a marked increase in the levels of Asf1b protein in tumoral versus normal cells (FIG. 6A). Quantitative RT-PCR analysis revealed a 2.8-fold increase in the levels of Asf1b mRNA in tumoral mammary cells indicating that Asf1b protein expression is regulated in part at the level of transcription (FIG. 6B). In contrast, the inventors did not observe significant changes in Asf1a protein or mRNA levels. Interestingly, expression of Asf1b paralleled the proliferative status of tumoral cells as assessed by CAF-1 p60 in Western blot (FIG. 6A) or CAF-1 p150 staining in immunofluorescence (FIGS. 6C and 5B). A closer analysis by immunofluorescence microscopy revealed that, in individual cells, Asf1b staining perfectly matched CAF-1 p150 staining, while in contrast, Asf1a did not (FIGS. 6D and 5B). The inventors thus conclude that Asf1b expression directly correlates with the proliferative status of mammary cells.

### Asf1a Versus Asf1b Depletion: Distinct Effects on Cellular Fate

The differential expression pattern of Asf1a and Asf1b in relation to proliferation prompted inventors to examine whether there could be different requirements for each Asf1 isoform. For this, they knocked-down Asf1a and Asf1b, individually or together, in human U-2-OS cells by RNA interference. They first verified the specific depletion of one or the other isoform by Western blot (FIG. 7A). While a single band corresponding to Asf1b or Asf1a remained visible after the single depletion of Asf1a or Asf1b respectively, the two bands corresponding to Asf1a and Asf1b isoforms disappeared in the double knockdown as seen by Western blot analysis (FIG. 7A). Importantly, while knockdown of both iso forms produced a strong accumulation of cells in S phase as shown previously, cell cycle profiles by FACS analysis did not change significantly after depletion of Asf1a or Asf1b alone (FIG. 7A).

Since Asf1 has been implicated in the regulation of transcription in yeast and Drosophila, the inventors examined the impact of each Asf1 iso form on transcription genome-wide. Using RNA extracted from cells depleted of the different Asf1 isoforms, the inventors performed a transcriptome analysis on a GeneChip Human Genome U133 Plus 2.0 Array (Affymetrix). Depletion of Asf1a, Asf1b or Asf1(a+b) was effectively detected under our hybridization conditions (FIG. 8B) and by Q-RT-PCR (FIG. 8A). The inventors observed that Asf1b depletion led to a slight but reproducible upregulation of Asf1a relative to the mock depletion, suggesting that a compensating mechanism was at play (FIG. 8B). In contrast, Asf1a depletion did not significantly alter Asf1b levels. The changes observed by the Affymetrix microarray analysis were recapitulated by quantitative-RT-PCR in 3 independent experiments for a set of selected genes (FIGS. 8A and 8C). Remarkably, the inventors did not find any obvious conservation of transcriptomic data with the one obtained in yeast suggesting that, beside sharing common molecular properties, human Asf1 isoforms and yeast Asf1 might have distinct functions. Interestingly, the Venn diagram showed that while the highest proportion of affected genes (2151) is shared between the three different siRNA conditions, Asf1b has the highest contribution to the overall changes observed in Asf1(a+b) depletion (FIG. 7B). This is consistent with the heatmap representation of the differentially expressed genes. Aware that the effects detected are modest compared to those observed for known transcription factors, the inventors performed a Gene Ontology (GO) analysis on the differentially expressed genes. Each of their siRNA conditions affected various functional classes of genes further emphasizing a clear distinct transcriptional signatures for Asf1a and Asf1b. The class of genes linked to cell proliferation significantly stood out in Asf1b depletion, supporting the importance of Asf1b in cell proliferation (FIG. 9).

The inventors then investigated in closer details the effects of Asf1a/b depletion at the cellular level by immunofluorescence microscopy, in the U-2-OS model cell line (FIG. 7), and in two breast cancer cell lines: Hs578T cells (FIGS. 17 and 18) and MDA-MB-231 cells (FIG. 19) in which they could obtain a significant depletion of Asf1 isoforms (FIGS. 17 and 19). Intriguingly, Asf1b depletion alone led to a remarkable and reproducible increase in the number of aberrant nuclear structures including altered nuclear morphology (3 to 10 times more than in the control siRNA) and micronuclei formation (FIGS. 7D-E, 18A-B and 19C). In addition, Asf1b depletion also increased the number of internuclear DNA bridges (FIGS. 18C and 19C). Quantification of the number of altered nuclei as well as the number of micronuclei in U-2-OS cells depleted with two independent sets of siRNA confirmed the specificity of the phenotype observed upon Asf1b depletion only (FIG. 7E). The expression pattern of lamin A, a marker of the nuclear periphery, was specifically altered in Asf1b-depleted cells suggesting the presence of an abnormal nuclear lamina in the lobulated nuclei (FIGS. 7F and 18C), not observed upon Asf1a depletion. The inventors then assayed the ability of cells depleted of the different Asf1 iso forms to undergo “unlimited” division. For this, they performed a Colony Formation Assay (CFA) ((Franken et al., 2006) on HeLa cells and mammary tumoral Hs578T cells transfected with control, Asf1a, Asf1b or Asf1(a+b) siRNAs. In this assay, a decrease in the number of colonies reflects either impaired proliferation, or increase in cell death or both. They observed a striking difference in the number of colonies obtained after the single Asf1 isoform depletions underscoring a distinct impact of Asf1a and Asf1b on proliferation (FIG. 7C and 18D). Asf1b-depleted cells formed fewer colonies than the control cells suggesting that the absence of Asf1b prevented continued proliferation. These results were confirmed in U-2-OS cells (FIG. 7G) and MDA-MB-231 cells (FIG. 19D). Importantly, given the decrease in a number of genes required for proliferation in Asf1b depleted cells, but not Asf1a, in transcriptomic data (data not shown), the effects observed upon Asf1b depletion most likely reflect an acute effect on proliferation leading to cell death as a consequence. This is further supported by transcriptomic data in which the inventors could not find any bias towards genes involved in cell death in Asf1b depleted cells. Collectively, the depletion analysis underscored distinct functions of Asf1 isoforms with an importance of Asf1b for proliferation.

Asf1b Correlates with Proliferation in Breast Tumor Samples

To assess the relevance of the inventors' findings connecting Asf1b with proliferation in a physiological context, they analyzed a selection of cryopreserved breast carcinoma samples collected in 1995 at the Institut Curie. Table 1A (here under), provides the patients' and tumor characteristics. They focused on node-negative and metastasis-free invasive breast carcinoma of a size that permitted primary conservative tumorectomy (median 18 mm; range 6-50 mm). The standard treatment received by the patients at the Institut Curie for such localized breast cancers was tumor excision with radiotherapy. However, adjuvant systemic therapy can increase the chance of long-term survival and determining which patients with localized breast cancers would benefit from these treatments is a current challenge. Thus, new classifiers could provide better guidelines for the administration of adjuvant chemotherapy. The inventors therefore measured Asf1a, Asf1b, CAF-1 p60 and CAF-1 p150 mRNA expression levels by quantitative RT-PCR in 86 breast tumor samples and normalized the expression levels to the known reference gene RPLPO (de Cremoux et al., 2004). For statistical analysis they retained data that fulfilled their amplification quality criteria (reproducible duplicates, consistent primer efficiency between samples).

TABLE 1A
Description of the samples from patients of 1995 with small breast tumors.

| Age | Median: 53 (range: 26-70) | | Size classification | T0/T1 | 62% | ER | (+) 86%/(−) 14% | |
|---|---|---|---|---|---|---|---|---|
| Menopaused | Yes | 54% | | T2 | 38% | PR | (+) 69%/(−) 31% | |
| | No | 46% | Tumor size (mm) | Median: 18 (range: 6-50) | | Ki67 | <=15 | 52% |
| Histological | ductal | 88% | Mitotic index | Median: 8 (range: 0-105) | | | 15-40 | 24% |
| type | lobular | 9% | Grade EE | I | 33% | | >40 | 24% |
| | papiliary | 1% | | II | 42% | Adjuvant | No | 93% |
| | tubular | 1% | | III | 25% | chemotherapy | Yes | 7% |

First, the inventors studied the correlation between the levels of each Asf1 iso form and that of another proliferation marker such as CAF-1 subunits (Polo et al., 2004) or Ki67 (Schonk et al., 1989). Asf1a levels only weakly correlated with that of Asf1b, CAF-1 p60 or CAF-1 p150 and did not correlate with Ki67. In contrast, Asf1b levels significantly correlated with p60 (r=0.7; p<10⁻⁹), p150 (r=0.6;)p<10^{-10) and Ki}67 (r=0.5; p<10⁻⁶), which again demonstrated its important link with cell proliferation (Table 1B and FIG. 10A). The inventors then investigated the correlation of Asf1a, Asf1b, CAF-1 or Ki67 levels with clinical parameters to evaluate a potential diagnostic value. They found a high significant positive correlation of Asf1b levels, but not Asf1a, with the tumor size (p=0.0063), the number of mitotic cells (p<10⁻⁵), and the grade of the tumor (p<10⁻⁵) (FIG. 11A). Notably, Asf1b proved even more significant than the other proliferative markers p60 and Ki67 (FIG. 11A). Table 1B (here under) summarizes all correlations. Taken together, the present observations put forward Asf1b as a new proliferation marker of clinical interest and prompted the inventors to examine its prognostic value in the context of breast cancer.

TABLE 1B
Comparison of Asf1a, Asf1b, CAF-1 p60, CAF-1 p150 and Ki67 between
multiple groups of prognostic factors.

| | Asf1a | | Asf1b | | p60 | | p150 | | Ki67 | |
|---|---|---|---|---|---|---|---|---|---|---|
| | N | p-value | N | p-value | N | p-value | N | p-value | N | p-value |
| Clinicopathological | | | | | | | | | | |
| factors | | | | | | | | | | |
| Age | | 0.70 | | 0.36 | | 1.0 | | 0.79 | | 4.7 × 10−3 |
| <=50 | 34 | | 53 | | 44 | | 53 | | 53 | |
| >50 | 21 | | 32 | | 31 | | 33 | | 33 | |
| Tumor size | | 0.06 | | 6.3 × 10−3 | | 0.028 | | 0.34 | | 6.6 × 10−3 |
| no tumor/T1a | 33 | | 53 | | 46 | | 53 | | 53 | |
| T2a | 22 | | 32 | | 29 | | 33 | | 33 | |
| Pathological Tumor | | 0.18 | | 2.1 × 10−4 | | 0.07 | | 0.17 | | 0.013 |
| <=20 mm | 38 | | 59 | | 51 | | 59 | | 59 | |
| >20 mm | 17 | | 26 | | 24 | | 27 | | 27 | |
| Number of mitosis | | 0.07 | | 6.3 × 10−6 | | 1.4 × 10−3 | | 1.2 × 10−3 | | 2.3 × 10−4 |
| <=10 | 31 | | 53 | | 44 | | 53 | | 53 | |
| >10 | 24 | | 32 | | 31 | | 33 | | 33 | |
| Grade EE | | 0.06 | | 1.2 × 10−6 | | 9.5 × 10−3 | | 0.04 | | 5.4 × 10−5 |
| I | 18 | | 29 | | 25 | | 29 | | 29 | |
| II | 24 | | 34 | | 29 | | 35 | | 35 | |
| III | 13 | | 22 | | 21 | | 22 | | 22 | |
| | Asf1a | | Asf1b | | p60 | | p150 | | Ki67 | |
| | r | p-value | r | p-value | r | p-value | r | p-value | r | p-value |
| Corrections with | | | | | | | | | | |
| other markers | | | | | | | | | | |
| CAF-1 p60 | 0.42 | 2.5 × 10−3 | 0.66 | 2.4 × 10−10 | — | — | 0.84 | p < 2.2 × 10−16 | 0.26 | 0.026 |
| CAF-1 p150 | 0.53 | 2.6 × 10−5 | 0.64 | 3.6 × 10−11 | 0.84 | p < 2.2 × 10−16 | | | 0.21 | 0.054 |
| Ki67 | 0.02 | 0.86 | 0.52 | 3.6 × 10−7 | 0.26 | 0.026 | 0.21 | 0.054 | | |
| Asf1a | | | 0.32 | 0.017 | 0.42 | 2.5 × 10−3 | 0.53 | 2.6 × 10−5 | 0.02 | 0.86 |
| Asf1b | 0.32 | 0.017 | | | 0.66 | 2.4 × 10−10 | 0.64 | 3.6 × 10−11 | 0.52 | 3.6 × 10−7 |

(Upper part) Correlations between the indicated genes and clinicopathological factors. N: number of samples included in the statistical analysis for each gene. Significant p-values (≦0.05) are noted in bold. (Lower part) Correlations between the genes. r: Pearson coefficient of correlation. Significant p-values (≦0.05) are noted in bold.

### Asf1b has a Prognostic Value in Breast Cancer

The inventors first investigated the relationships between Asf1b levels and disease outcome, as determined by the disease free interval, the overall survival and the occurrence of metastasis. Since Asf1a did not show significant correlation with any of the clinical markers studied, they did not consider it in this analysis. At 10 years, the overall survival, the distant recurrence and the disease progression rates were 90 [83-97], 87 [80-95] and 70% [61-81], respectively. They determined a cut-off value of 0.7 for Asf1b mRNA levels which divided patients into two groups: one with low Asf1b levels (67% of patients with Asf1b≦0.7), and the second with high Asf1b levels (>0.7) which was significantly associated with disease progression (p=0.017, Relative Risk (RR)=2.3 [1.1-4.8]) in univariate analysis (FIG. 11B). Moreover, higher Asf1b levels significantly associated with shorter overall survival (p=0.01, RR=6.3 [1.3-31.3]) and an increased occurrence of distant metastasis (p=0.0002, RR=7.8 [2.1-28.3]) (FIGS. 11B and 10B) further underlining the prognostic value of Asf1b. At 10 years, 98% [93-100] of the patients with low Asf1b expression had not developed metastasis compared to 66% [50-87] of the patients with high Asf1b levels.

In a second independent set of 71 breast cancer samples collected in 1996 at the

Institut Curie, the inventors confirmed the obtained results. Table 2 provides the patients' and tumor characteristics for this set.

TABLE 2
Description of the samples from patients of 1996 with small breast tumors.

| Age | Median: 55 (Range: 30-69) | | Size classification | T0/T1 | 66% | ER | (+) 80%/(−) 20% | |
|---|---|---|---|---|---|---|---|---|
| Menopaused | Yes | 37% | | T2 | 34% | PR | (+) 77%/(−) 23% | |
| | No | 63% | Tumor size (mm) | Median: 20 (Range: 8-35) | | Ki67 | <=15 | 44% |
| Histological | ductal | 74% | Mitotic index | Median: 5 (Range: 0-120) | | | 15-40 | 25% |
| type | lobular | 16% | Grade EE | I | 30% | | >40 | 31% |
| | papiliary | 1% | | II | 48% | Adjuvant | No | 93% |
| | tubular | 9% | | III | 22% | chemotherapy | Yes | 7% |

Asf1b expression correlates with the tumor characteristics, in particular the mitotic index (p<10⁻³), the tumor grade (p=0.002) and the hormone receptor status (p=0.01) (FIG. 14). In addition, as shown in FIG. 15, they confirmed that Asf1b expression is a prognosis marker. They determined a cut-off value of 0.27 for Asf1b mRNA levels which divided patients into two groups: one with low Asf1b levels (41% of patients with Asf1b≦0. 27), and the second with high Asf1b levels (>0. 27) which was significantly associated with disease progression (p=0.045, Relative Risk (RR)=2.7 [1.0-7.4]) in univariate analysis (FIG. 15). Moreover, higher Asf1b levels significantly associated with shorter overall survival (p=0.018, RR=2.9 [1.2-7.2]) and with an increased occurrence of distant metastasis (p=0.007, RR=9.9 [1.3-76.7]) (FIG. 15) further underlining the prognostic value of Asf1b.

Next, the inventors compared the prognostic value of Asf1b with CAF-1 p60, CAF-1 p150 and HP1α (De Koning et al., 2009) in multivariate analysis adjusted for known prognostic factors and for the genes of interest. They found that only high CAF-1 p60 expression was an independent prognostic factor for disease progression (p<10⁻⁴, RR=5.5 [2.5-11.9]) and decreased overall survival (p<10⁻³, RR=12.9 [2.6-64.2]) (FIG. 13A). Their observations thus confirm that CAF-1 is not only of interest for diagnosis (Polo et al., 2004) but also show its relevance for the prognosis of breast cancer. Notably, Asf1b stood out as the only independent prognostic factor for the metastasis free interval (p<10⁻³). High Asf1b levels are associated with a higher risk of developing distant metastasis (RR=7.1 [2.0-26.0]) (FIG. 13A). In the set of tumor samples from 1996, they found menopausal status (p=0.013, RR=4.2 [1.4-12.8]) and Asf1b mRNA levels (p=0.024, RR=5.7 [1.3-25.7]) as independent prognostic markers for metastasis free interval (FIG. 20), therefore confirming data obtained in the first series of tumor samples. Interestingly, together with menopausal status, Asf1b expression levels also significantly predicted disease progression (FIG. 20). Thus, they demonstrate for the first time that Asf1b is a new proliferation marker of prognostic value in breast cancer that is highly predictive for the occurrence of metastasis.

### Asf1 Levels in Breast Tumor Subtypes

In breast cancer, expression-profiling studies have helped to distinguish different subtypes of tumors according to a specific expression profile (Sotiriou and Piccart, 2007) defining the following molecular classes: luminal-A cancers and luminal-B cancers, which are predominantly Estrogen Receptor (ER)-positive; basal-like cancers, which mostly correspond to ER-negative, Progesterone Receptor (PR)-negative and HER2-negative tumors; and HER2-overexpressing cancers corresponding to tumors with amplification of the ERBB2 gene. Importantly, these molecular subgroups have distinct clinical outcomes and responses to therapy, with the basal-like tumors and HER2 positive tumors having a more aggressive clinical picture (Sotiriou and Piccart, 2007). Taking advantage of an available transcriptome database derived from breast tumor samples of cryopreserved tissues selected from the Institut Curie, the inventors examined Asf1 levels in specific subtypes of breast tumors. Asf1a mRNA levels were similar in normal breast samples, luminal tumors (luminal and micropapillary) and basal-like (BLC) subtypes, and only significantly increased in the medullary basal-like (MBC) subtype which has an inflammatory stroma (FIG. 13B). In contrast, Asf1b mRNA levels were low in normal breast tissue and significantly increased in all breast tumor subtypes (FIGS. 13B). Interestingly, the inventors found the highest expression levels for Asf1b in the BLC and MBC subgroups, corresponding to the basal-like highly proliferative tumors (FIG. 13B). Thus, Asf1b expression levels showed a clear association with the proliferation rate and aggressiveness of distinct breast cancer subtypes.

Identification of Novel Structural Differences between Asf1a and Asf1b Isoforms and their Functional Implications

While Asf1 exists as a single isoform in Fungi (eg Saccharamyces cerevisiae), the inventors uncovered a major duplication event leading to the clear distinction of Asf1a and Asf1b isoforms in Amniotes (data not shown). They therefore investigated the structural divergence between these two iso forms to try and assign distinct functions to each individual Asf1 iso form. Evidence suggesting a potential difference in the functions of the two mammalian Asf1 isoforms came both from studies in yeast and mammalian cells. The depletion of Asf1 in yeast is best rescued by human Asf1a for defects in the DNA-damage response, while Asf1b best compensates for the growth defects and the sensitivity to replication stress (Tamburini et al., 2005). In mammalian cells, Asf1a specifically interacts with HIRA and is required together with HIRA for senescence-associated cell-cycle exit (Zhang et al., 2005). Recent evidence also suggests that Asf1a could play an important role in the regulation of H3K56 acetylation levels in human cells and in transcriptional activation mediated by H3.3 incorporation together with HIRA (Das et al., 2009; Yang et al., 2010; Yuan et al., 2009). In contrast, as shown herein, Asf1b is the Asf1 isoform essential for proliferation.

However to date, the structural basis for such a possible divergence in function is poorly understood. The interaction domain of Asf1 with histones H3-H4 is highly conserved between Asf1a and Asf1b isoforms (English et al., 2006; Natsume et al., 2007), as well as its hydrophobic core interacting with the B-domain of HIRA or CAF-1 p60 (Malay et al., 2008; Tang et al., 2006). In addition, both Asf1a and Asf1b are known to interact with both H3.1 and H3.3 histone variants (English et al., 2006; Natsume et al., 2007; Tagami et al., 2004). Thus, Asf1 is not the primary factor that discriminates between the histone variants, and the distinct functions of Asf1 isoforms are most probably not a consequence of a preferential interaction with a given histone variant. This is consistent with the fact that among the four residues that differ between the two histone variants, three are clustered on the opposite site of the Asf1 binding site and the fourth is located in the N-terminal tail which most likely does not interact with Asf1 (English et al., 2006; Natsume et al., 2007). It is therefore intriguing to realize that despite the high degree of similarity between the two human Asf1 isoforms (71% of identity), the small divergence observed in the first 30 amino-acids of the sequence, and the different C-terminal domains (aa 156-202) may be sufficient to elicit specific interactions/functions with other histone chaperones, as suggested by swapping experiments of the Asf1a and Asf1b N- and C-terminal domains (Tang et al., 2006).

Here, the inventors determined the divergent amino-acids between the consensus sequences of Asf1a and Asf1b isoforms in Amiotes (FIG. 21). Since the distinct amino-acids are dispersed along the Asf1 sequence (FIG. 21A), they then visualized the localization of these amino-acids on the known structure of the human Asf1a N-terminus (aa 1-155) (Mousson et al., 2005). They found a striking clustering of these amino-acids in 3D at the top and bottom of Asf1, defining two potential regions mediating the specificity of the Asf1a and Asf1b isoforms (FIG. 21B). Importantly, these two regions are distinct from the previously characterized interaction domains of Asf1 with histones (FIG. 21B) and with HIRA or CAF-1 p60 (FIG. 21B) which are highly conserved between Asf1a and Asf1b. They hypothesize that these two regions could reveal additional specific interaction of Asf1a/b with the known histone chaperones HIRA or CAF-1 p60, or with other partners yet to be identified. Interestingly, using specific antibodies against Asf1a and Asf1b as previously described herein, they performed an immunoprecipitation of Asf1a or Asf1b from Hela S3 total cell extracts. While they confirmed the preferential interaction of Asf1a with HIRA, they uncovered for the first time a preferential interaction of Asf1b with CAF-1 p60 (FIG. 21C).

Since the C-terminal extention of Asf1 is unfolded in the isolated proteins, they restricted their analysis to the N-terminal domain of human Asf1. Nevertheless, it is possible that the C-terminus adopts a well-defined structure in the presence of partners. In addition, the phosphorylation imposed on the C-terminal domain of Asf1 isoforms could also participate in the regulation of their specific functions, as suggested recently for a differential regulation of the stability of Asf1 isoforms (Pilyugin et al., 2009). One could envisage that these divergences in the sequence/structure of Asf1 iso forms on the C-terminus could also provide the specificity towards new interaction partners, as was the case for the Asf1a-HIRA interaction or for the recently identified interaction between the Varicella-Zoster Virus (VZV) immediate-early 63 protein (1E63) and Asf1a isoform.

Inhibition of Chromatin Assembly with the B-Domain of HIRA

Among its conserved functions, the histone chaperone Asf1 synergizes with CAF-1 and HIRA in the replication-coupled and replication-independent nucleosome assembly pathways, respectively, rather than acting as a deposition factor on its own in vivo. Asf1 interacts with the B-domain of HIRA or CAF-1 p60 through a conserved hydrophobic groove at a site located on the opposite side of the one involved in its interaction with H3-H4 (English et al., 2006; Natsume et al., 2007). This explains how the histones can be handed over from one histone chaperone to another in order to promote chromatin assembly. One could hypothesize that a peptide of the B-domain present in excess could block the interaction of Asf1 with HIRA and/or CAF-1 p60, and thus inhibit the function of the histone chaperone Asf1 in chromatin assembly.

The inventors therefore decided to investigate if the B-domain of HIRA could inhibit chromatin assembly promoted by HIRA and Asf1. For this, they decided to use a cell-free system enabling chromatin assembly derived from Xenopus laevis eggs. These egg extracts contain all factors essential for chromatin assembly during the rapid rounds of DNA replication that occur in early development. When depleted of a putative histone chaperone or with an excess of a putative inhibitory peptide, they lose their ability to support nucleosome assembly, which can be restored with add-back experiments. This approach was successfully used to demonstrate that CAF-1 and HIRA are true deposition factors in vivo, promoting chromatin assembly dependent and independent of DNA synthesis respectively (Quivy et al., 2001; Ray-Gallet et al., 2002). The inventors therefore cloned the B-domain of X. laevis HIRA in a vector allowing its expression as a peptide with a GST tag at its end. In addition, they cloned a mutated form of the B-domain mutated at position I461D which has been shown to no longer interact with Asf1 (Tang et al., 2006). They verified by GST pull-down that the GST-B-domain of HIRA interacts in vivo with Asf1 from X. laevis (FIG. 22A). Importantly, the mutated form of the B-domain of HIRA (I461D) does not interact with Asf1, consistent with previous data (Tang et al., 2006). They then wondered if addition of an excess of the B-domain of HIRA could inhibit chromatin assembly independent of DNA replication in the X. laevis system. They therefore added the GST-B-domain of HIRA to the nucleosome assembly reaction. They observed a striking decrease in the amount of supercoiled plasmid (1 form) (FIG. 22B) suggesting that nucleosome assembly is impaired in the presence of the B-domain of HIRA. Importantly, this dominant negative effect of the B-domain is working through its interaction with Asf1 since addition of the mutated GST-B-domain I461D which does not interact with Asf1 had no effect on nucleosome assembly (FIG. 22B). In addition, they could rescue the nucleosome assembly reaction by adding excess of Asf1 proteins to the reaction (data not shown).

In conclusion, these data show that the use of the B-domain peptide can inhibit chromatin assembly independent of DNA synthesis by inhibiting the interaction of Asf1 with HIRA. They hypothesize that the excess of B-domain peptide titrates Asf1 proteins away which can then no longer interact with HIRA, resulting in an inhibition of chromatin assembly. This is consistent with the fact that addition of an excess of Asf1 proteins can rescue the nucleosome assembly. A similar strategy could therefore be used to inhibit Asf1 interaction with CAF-1. In particular, given the preferential interaction of Asf1b with CAF-1 (FIG. 21 C) and given the existence of specific structural regions that could mediate this preferential interaction of Asf1b with CAF-1 (FIG. 21B), one could imagine that the B-domain peptide could be refined to specifically inhibit the interaction of Asf1b with CAF-1. In addition, the peptide could also be designed to inhibit the interaction with histones together with the interaction with CAF-1, potentially leading to major proliferation defects (FIGS. 7, 18 and 19).

### Discussion

While yeast presents a single form of the Asf1 histone H3-H4 chaperone, in many multicellular organisms, including plants or mammals, there are two distinct iso forms whose specific individual functions have remained unexplored. The inventors investigated their respective implication in cell proliferation. In cultured cells, they reveal a unique proliferation-dependent expression pattern of Asf1b, not shared by Asf1a, that enables to distinguish tumoral from non tumoral derived breast cancer cells. Depletion of Asf1b shows the prominent role of this iso form for cell proliferation, with a distinct transcriptional impact genome-wide and cellular defects reminiscent of aging phenotypes. Moreover, using a selection of samples from early stage breast tumors derived from patients, they demonstrate for the first time the clinical relevance of Asf1b as a proliferation marker of prognostic value in early stage breast cancers.

A Distribution of Labour between Asf1 Isoforms for Distinct Proliferation Status

Using various model cell lines, the inventors found that the two Asf1 isoforms are expressed in a distinct manner. While Asf1a levels remain unchanged in cycling, quiescent or senescent cells, Asf1b levels directly reflect the proliferation capacity of the cells both at the protein and RNA levels. Remarkably, when exploring data from the Gene Expression Omnibus database (GEO, NCBI) they found that the proliferation-dependent expression of human Asf1b was conserved in other human cell types, such as in the human T98G glioblastoma cancer cells arrested by serum deprivation (GEO accession number: GDS911), and also extends to other organisms such as mouse (GDS575), thus opening interesting avenues for genetic studies. Interestingly, the levels of Asf1 isoforms may also vary in a distinct manner upon differentiation (GDS586), as another form of cell cycle exit. Thus, while Asf1 iso forms share molecular and biochemical properties as histone H3-H4 chaperones, as exemplified by their overlapping functions during replication (FIGS. 7A and 7B), the difference between these iso forms could reside partly in their capacity to be uniquely regulated. In this respect, the finding that Asf1b is a direct transcriptional target of E2F1 would find a physiological application in the present results to explain the proliferation-dependent regulation of Asf1b. However, given that both Asf1a and Asf1b genes possess putative binding sites for the transcription factors E2F, additional aspects should impart on this regulation. The present study in multicellular organisms showing the specific regulation of Asf1a and Asf1b levels in distinct cellular contexts as a means for a distribution of labour between the two Asf1 isoforms, opens up avenues to examine how common properties can be exploited in different physiological contexts.

### Functional Importance of Asf1 Isoforms

The genome-wide transcriptome analysis coupled with the analysis of the effects caused by the depletion of Asf1 isoforms shows that, while Asf1a and Asf1b can partly compensate for each other (FIG. 7A), these two isoforms have a distinct role in mammalian cells. Importantly, most of the effects observed in the transcriptome analysis were relatively small compared to the depletion of transcription factors. This together with the preliminary ChIP-on-chip data supports the view according to which Asf1 iso forms may rather play an auxiliary role in transcriptional regulation, while their main function is to regulate the dynamics of the histone pool. The latter one could however show potential specificities and regulations depending on cellular context. It is tempting to consider that despite the high degree of similarity between the two human Asf1 isoforms (71% of homology as shown in FIG. 1A), the divergence observed in the first 30 amino-acids of the sequence, together with the different C-terminal part (156-202 amino-acids) could contribute to the distinct regulations and functions of the two Asf1 iso forms. In this respect, given that the available information on structural organization of Asf1 focus on the conserved N-terminus, future studies aiming to characterize the 3-dimensional organization of the C-terminus should be revealing. This may provide a particular docking site for new, yet to be identified, interaction partners, as it is the case for the Asf1a-HIRA interaction.

Asf1b appears to be most critical for proliferation (FIGS. 7B to 7F). In proliferating cells, Asf1b would be best able to handle the pool of replicative histone H3.1 thereby acting as the prominent histone acceptor/donor during DNA replication. This would be consistent with its major contribution to the defects observed upon knockdown of both Asf1 iso forms (FIG. 7B). While we cannot formally exclude that Asf1b could potentially directly upregulate genes related to DNA replication (FIG. 9, list for Asf1b depleted cells), such transcriptional changes could simply represent a indirect effect enabling to compensate replication defects. Furthermore, when cells enter into a non-dividing state, the amount of the H3.1 replicative histone variant available drops. Asf1a would then suffice to handle the remaining pool of histones consisting mainly of H3.3 during all DNA metabolic processes. While some preferential interactions with a given H3 variant are plausible, structural studies will be needed to explore whether this is dictated by the binding properties of the partners or simply a reflection of availability of given H3 variants. Interestingly, Asf1b depletion led to a slight upregulation of Asf1a at the RNA level, and did not give any obvious phenotype during S phase by flow cytometry analysis (FIG. 7A), which would favor the latter option. In this context, the upregulation of Asf1a in cells depleted of Asf1b could represent a compensatory mechanism to allow normal S phase progression. Notably, in addition to its effect on cell proliferation, Asf1b depletion also led to an increased number of aberrant nuclear structures which resemble those observed in Hutchinson-Gilford progeria syndrome (HGPS) cells and in cells depleted of the histone chaperone RbAp48. RbAp48 is a histone chaperone found in a complex together with Asf1a and Asf1b and belongs to several chromatin-related complexes such as CAF-1, NURD or PRC2 complexes. Interestingly, the downregulation of RbAp48 expression in HGPS cells was found to participate in the formation of ageing-associated chromatin defects via loss of the integrity of the NURD complex. Intriguingly, in Asf1b depleted cells, but not Asf1a, RbAp48 gets downregulated. It is thus tempting to speculate that Asf1b may also act as a chaperone of RbAp48 to maintain levels required to prevent the appearance of premature aging-related chromatin defects. Having high levels of Asf1b would therefore confer a growth advantage to proliferating cells, in agreement with our observations that Asf1b levels are highly downregulated in primary senescent fibroblasts (FIG. 3A). Thus, not only is Asf1b expressed in a proliferation-dependent manner, but it is required for proliferation, and possibly prevents aging. These two aspects are both relevant for cancer progression.

### Asf1b as a Marker of Diagnostic and Prognostic Value for Breast Cancer

Using samples from 86 early-stage breast tumors with a >10 years follow-up, the inventors demonstrate for the first time the clinical relevance of Asf1b as a new prognostic factor in breast cancer. High Asf1b levels correlate with a poor overall survival rate, a decreased disease free interval and a higher occurrence of metastasis. By analyzing published transcriptomic data of the Oncomine database (Rhodes et al., 2004), they confirmed the prognostic value of Asf1b in breast cancer (FIG. 12A). Their current analysis extended to a second, independent set of patient samples further confirm the highly prognostic value of Asf1b and CAF-1 p60 compared to other markers. In addition, Asf1b levels also identify the aggressivity of breast tumor subtypes, being higher in the basal-like cancers (FIG. 13B), and also in the Erbb2 positive tumors. Multivariate analyses demonstrate that Asf1b levels predict the occurrence of metastasis better than any standard prognostic markers, while CAF-1 p60 proved here to be another prognostic factor with a better prediction value for the disease free interval and survival rates, consistent with recent data. Furthermore, the inventors found that beside a significant and consistent overexpression in breast cancers, Asf1b stands out in other types of malignancies, such as skin cancer, liver cancer, ovarian cancer, lung cancer, liposarcoma, gastric cancer, pancreatic cancer, bladder cancer, vulvar cancer, colon cancer and brain cancer (FIGS. 12B and 25). Asf1b was also found in a ‘cervical cancer proliferation cluster’ of 163 highly correlated transcripts which were overexpressed in cervical tumors with an unfavourable disease outcome. In contrast, Asf1a was not retrieved as a significant gene in these studies. The present results therefore lead us to propose that Asf1b represents a new proliferation marker of interest in a wide range of cancers which can be used as a classifier with a powerful prognostic value for metastasis occurrence.

Interestingly, proliferation appears to be a common driving force of several gene expression prognostic signatures such as MammaPrint (van't Veer et al., 2002), or the genomic-grade signature (Sotiriou et al., 2006), which aim at providing better predictions of clinical outcome than the traditional clinical standards (Wirapati et al., 2008). The inventors found that Asf1b correlates with prognosis in breast cancer transcriptomic data validating the Mammaprint signature (FIG. 12A) and that Asf1b belongs to a set of co-expressed proliferative genes with prognosis value in breast cancer. Remarkably, while most genes of this proliferation module are directly related to progression through cell cycle such as cyclins, replication factors, aurora kinases etc, Asf1b stands out as an interesting proliferation marker related to chromatin organization and histone dynamics. One could imagine that high Asf1b levels would confer an important chromatin plasticity, which is essential for the survival of cancer cells in a selective environment. The prognostic value of Asf1b could therefore bring some complementary information compared to the previously known prognostic markers. Combination of Asf1b with other selected markers related to chromatin organization such as HP1α (De Koning et al., 2009), and CAF-1 p60 may have a stronger clinical value in the most aggressive breast cancer subtypes such as basal-like tumors.

In conclusion, the present study enables to ascribe to the distinct Asf1 isoforms specific roles associated with different proliferation states. On the one hand, the proliferative predominent role of Asf1b would ensure to handle replicative histones via a replication-oriented function which could prevent aging. On the other hand, Asf1a would rather contribute to processes in which handling histones would connect to transcription/silencing or senescence. The proliferative role of Asf1b, culminates with a validation as a new proliferation marker of interest both in the context of model cell lines and tumor samples. Furthermore, the high Asf1b expression correlating with increased rates of disease progression and metastasis occurrence in small breast cancer, defines Asf1b as a new prognostic factor of clinical value. Future work should explore how to exploit these findings that highlight Asf1b as an attractive target for cancer treatment.

### Materials and Methods

### Cell Lines and Cell Culture

DMEM medium (GIBCO) was used for U-2-OS osteosarcoma (gift from J. Bartek, Copenhagen), HeLa cervical carcinoma (gift from M. Bornens, Paris), MCF7 and MDA-MB-231 breast adenocarcinoma cancer cell lines, MEMα medium (GIBCO) was used for BJ primary foreskin fibroblasts (CRL-2522, ATCC), RPMI medium (GIBCO) supplemented with 10 μg/mL insulin (Sigma) was used for HS478T breast cancer cells (gift from 0. Delattre, Paris) (Hackett et al., 1977), and DMEM medium (GIBCO) containing 3Ong/mL Epidermal Growth Factor (TEBU) was used for HS478Bst healthy mammary cells (ATCC) (Hackett et al., 1977). All media contain 10% FCS (Eurobio) and 10mg/mL penicillin and streptomycin (GIBCO). Glutamax-DMEM (Invitrogen) supplemented with 15% FCS was used to grow early passage (PD25) IMR90 human primary fibroblasts (ATCC) at 7.5% CO₂ and 3% 0₂. To obtain old (PD72) and senescent (PD80) cells, cells were passaged in a 1:4 regimen for additional population doublings where the new PD was calculated as PD=PD at plating+[In (#harvested/#seeded)/In2]. Cells were counted with the Z1 Coulter Particule Counter (Beckman coulter).

### Synchronization of Cells

BJ primary cells and U-2-OS osteosarcoma cells were synchronized in quiescence by incubation in a serum-free medium for 72 h, and MCF7 cells in medium containing 10 nM of the anti-estrogen IC1182780 (Fisher Bioblock Scientific) (Carroll et al., 2000) for 48 h. HeLa cells were synchronized with a double thymidine block, as follows: 16 h block in 2.5 mM thymidine (Sigma-Aldrich), 9 h release in 30 mM 2′-deoxycytidine (Sigma-Aldrich), and 16 h block in 2.5 mM thymidine. The G1/S, S, S/G2, and G1 samples were collected after a 0-, 4-, 8-, 14-h release in 30 mM 2′-deoxycytidine respectively. HeLa cells were treated with 100 ng/mL nocodazole for 15 h to obtain mitotic samples.

Cell synchronization was verified by flow cytometry, using cells fixed in 70% ethanol (−20° C.) and stained with propidium iodide (50 mg/mL in PBS containing 0.04 mg/mL RNase A). A BD FACScalibur (BD Biosciences) was used for signal analysis and analysis was carried with FlowJo (Tree Star Inc.) software.

### Plasmid Constructions for the Asf1 Recombinant Proteins and Antibodies

N-terminal fusions of the C-Terminal part of Asf1b (amino-acids 156-202) to a GST-tag and to a His-tag were generated by PCR cloning of the C-terminus of Asf1b (primers: 5′AGGTGCTAGAATTCAACATGGACAGGCTGGAGGCCATAG (SEQ ID No 23), 3′CAGGCTATCTCGAGTTATTAGATGCAGTCCATGGAGTTCTCAG (SEQ ID No 24)), insertion into the EcoRI/XhoI site of pGEX-4T-1(Novagen) and pET-30a (Novagen) respectively followed by verification by sequencing. N-terminal fusion of the C-Terminal part of Asf1a (amino-acids 156-204) to the His-tag was generated by PCR cloning of the C-terminus of Asf1a (primers: 5′AGGTGCTAGAATTCAACACAGAAAAACTGGAAGATG (SEQ ID No 25), 3′CAGGCTATCTCGAGTTATCACATGCAGTCCATGTGGGATTC (DEQ ID No 26)), insertion into the EcoRI/XhoI site of pET-30a (Novagen) and verification by sequencing.

### Plasmid Constructions for the Recombinant HIRA B-Domain Proteins

The HIRA B-domain from Xenopus laevis corresponding to amino acid 435 to 480 from HIRA (Accession number: AJ404369) was cloned into pGEX4T-1 (GE healthcare) and transformed in BL21(DE3) (calbiochem) in order to purify bacterially expressed GST HIRA B-domain recombinant protein (see below). The DNA sequence of the HIRA B-domain (1303-1440 pb) is the following: 5′-GGGGAAAGCTTGGAGGACATAAGAAAGAACCTCTTGAAAAAGCAAGTGGA GACACGAACAGCTGATGGACGGCGAAGGATCACTCCACTCTGCATTGCTC AGCTAGACACTGGGGACTTTTCCACAGCGTTTTTCAAT-3′ (SEQ ID No 27) and the protein sequence of the HIRA B-domain Protein (aa 435-480) is the following: GESLEDIRKNLLKKQVETRTADGRRRITPLCIAQLDTGDFSTAFFN (SEQ ID No 28).

The DNA sequence of the mutated HIRA B-domain I461D from Xenopus laevis that does not interact with Asf1 anymore is the following: 5′-GGGGAAAGCTTGGAGGACATAAGAAAGAACCTCTTGAAAAAGCAAGTGGAGAC ACGAACAGCTGATGGACGGCGAAGGGACACTCCACTCTGCATTGCTCAGCTAGA CACTGGGGACTTTTCCACAGCGTTTTTCAAT-3′ (SEQ ID No 29) and the protein sequence of the mutated HIRA B-domain I461D protein (aa 435-480) is the following: GESLEDIRKNLLKKQVETRTADGRRRDTPLCIAQLDTGDFSTAFFN (SEQ ID No 30). Of note, the protein sequence for the B-domain of human HIRA is the same (100% homology) with the one of Xenopus laevis.

### Antibodies

Rabbit polyclonal antibody raised against the full-length GST-Asf1a (antibody #28134) was described previously (Mello et al., 2002). An additional specific antibody against Asf1b was produced. For this, the C-terminal part (amino-acids 156-202) of Asf1b was cloned in a pGEX-4T-1 vector (Novagen) (see above). For the immunization of two rabbits (#18130 and #18143) (Agrobio), bacterially expressed GST-C-Term-Asf1b recombinant protein in E. Coli BL21 (DE3, Novagen) was used (Moggs et al., 2000), purified on glutathione beads (17-0756-01, GE Healthcare) and eluted with 10 mM glutathione according to the manufacturer's instructions. The specificity of both Asf1a and Asf1b antibodies was confirmed by Western blotting and immunofluorescence microscopy (FIG. 1). Since Asf1a and b migrate at different positions, a mix of the specific Asf1 antibodies was used for simultaneous detection of the two isoforms by Western blotting. For immunofluorescence studies, either highly purified Asf1a (#28134) or Asf1b (#18143) antibodies were used from sera (Agrobio). Table 3 compiles all primary antibodies with their source, reference, and dilutions for western blotting or immunofluorescence. In addition, the inventors used Rabbit polyclonal antibody against HIRA (Ray-Gallet et al., 2002) and against GST (Abcam ab9085) to reveal these proteins on FIG. 22.

TABLE 3

| | List of all primary antibodies used in this study. | | | | | | |
|---|---|---|---|---|---|---|---|
| | Company/ | Oder | Lot | | | WE | IF |
| Antibody | Reference | Number | Number | Species | | dilution | dilution |
| Asf1a | Mello et al., 2002 | — | #28134 | Rabbit polycional | | mix (a + b) | 1/2000 purified |
| | | | | | {close oversize brace} | | |
| Asf1b | This study | — | #18143 | Rabbit polycional | | 1/1000 each | 1/500 purified |
| α-Tubulin | Sigma | T9026 (DM1A) | 104K4800 | Mouse monocional | | 1/10 000 | — |
| β-actin | Sigma | A5441 (AC-15) | — | Mouse monocional | | 1/25 000 | — |
| CAF-1 p60 | Quivy et al., 2008 | — | #17019 | Rabbit polycional | | 1/1000 | — |
| CAF-1 p150 | Abcam | ab7655 | 588276 | Mouse monocional | | — | 1/1000 |
| CENP-A | Abcam | ab13939 | 628216 | Mouse monocional | | — | 1/250 |
| CyclinA | Santa Cruz | sc-751 | G0104 | Rabbit polycional | | 1/1000 | — |
| H4 | Upstate | 05-856 | JBC1361839 | Rabbit polycional | | 1/1000 | — |
| HJURP | Kato et al., 2007 | — | — | Rabbit polycional | | 1/1000 | — |
| HP1α | Euromedex | 2HP-2G9 | — | Mouse monocional | | 1/500 | — |
| LaminA/C | Ceil signaling | 2032 | 2 | Rabbit polycional | | — | 1/50 |
| Mom2 | BD Transduction | BM28 | 39289 | Mouse monocional | | 1/1000 | — |
| | laboratories | | | | | | |
| PCNA | DAKO | M0879 (PC-10) | 00026418 | Mouse monocional | | 1/2000 | — |
| RbAp48 | Abcam | ab1766 | 8829 | Rabbit polycional | | 1/1000 | — |

Company, as well as the order number, the lot number, the species and the dilutions for western blotting (WB) and immunofluorescence (IF) are provided for each antibody. Since Asf1a and Asf1b migrate at different positions in western blot, a mix of the specific Asf1 antibodies has been used in Western blot to recognize simultaneously the two isoforms. The specific purified antibodies against Asf1a or Asf1b were used separately in immunofluorescence.

siRNA and Transfections

U-2-OS, Hs578T and MDA-MB-231 cells were transfected in an antibiotics-free medium for 48 h with 100 nM siRNA using Oligofectamine reagent (Invitrogen) and optimem 1 medium (GIBCO) according to manufacturer's instructions. siRNA sequences were used against Asf1a (GTGAAGAATACGATCAAGTdTdT, SEQ ID No 13), Asf1b (CAACGAGTACCTCAACCCTdTdT, SEQ ID No 14), siControl (GCGCGCTTTGTAGGATTCGdTdT, SEQ ID No 15) and siGFP

(CACTTGTCACTACTTTCTCdTdT, SEQ ID No 16) (Dharmacon) as in (Groth et al., 2007; Groth et al., 2005) of siRNAs against Asf1a and Asfb at a final concentration of 50 nM each for the double depletion of Asf1(a+b).

### Colony Formation Assay

Hela US-2-OS, Hs578T and MDA-MB-231 cells were transfected with siRNA againstAsf1a, Asf1b, Asf1(a+b) or GFP or with a control siRNA as above. 24 h after transfection, 1000-2000 cells were plated as a single-cell suspension in 6 cm dishes, allowed cells to grow under normal conditions for 8-12 days before staining with 0.1% Coomassie Brilliant Blue R-250 (Bio-Rad) dissolved in 50% methanol, 15% acetic acid and counted colonies using an automatic counting colony counter pen. The mean plating efficiency (0.38) and the surviving fraction were determined as in (Franken et al., 2006).

### Western Blotting

For total extracts, lysed cells were processed in Laemmli sample buffer (LSB) 1× (62,5mM Tris HCl pH=6.8, 10% glycerol, 2% SDS, 0.002% bromophenol blue and 100mM DTT) as in ((Martini et al., 1998). Memcode Protein Stain Kit (Thermo Scientific) was used to detect proteins transferred on nitrocellulose membranes. Table 3 lists primary antibodies. Secondary antibodies conjugated with Horseradish peroxidase (HRP) (Interchim) were used and revealed signal by chemiluminescence substrate from Pierce (SuperSignal West Pico or SuperSignal West Femto). For quantification, the inventors performed acquisition of the chemiluminescence signal on a ChemiDoc XRS (BioRad) geldoc, and quantification of the intensity of the bands with Quantity One 4.6.6 software. It was checked that the signal response is in a linear range using dilution series. Values obtained for Asf1a or Asf1b levels were normalized to the levels of a-tubulin, or to the Memcode (Invitrogen) protein staining in the case of the mammary cell lines.

### Immunofluorescence Microscopy

Cells grown on coverslips, fixed in 2% paraformaldehyde, and permeabilized in PBS containing 0,2% Triton X-100, were processed as in (Martini et al., 1998). For lamin A staining, a pre-extraction step was performed to remove soluble proteins. Briefly, cells were washed with CSK, extracted with CSK 0.5% Triton X-100 and rinsed with CSK and PBS before fixation as described above. Cross-absorbed Alexa-488 or Alexa-594 conjugated secondary antibodies (Molecular probes-Invitrogen) were used to detect primary antibodies (Table 3). Images were acquired with a DM600 (Leica) upright widefield epifluorescence microscope (63× objective/NA 1.32 or 40× objective/NA 1.0) piloted with Metamorph software and equipped with a chilled CCD camera (CoolSnap Hq2, Photometrics). Identical settings and the same contrast adjustment were applied for all images to allow accurate data comparison, except for LaminA staining on FIG. 18C which was specifically enhanced in Asf1b depleted cells in order to visualize the DNA bridges. For brightness and contrast adjustment, Adobe Photoshop CS3 (Adobe) was used. For quantitative analysis, a minimum number of n=100 nuclei were counted per experiment.

### Primers

For analysis of the 86 and 71 breast tumor samples from 1995 and 1996, the following primers were used:

Asf1a Forward:
(SEQ ID No 1)
CAGATGCAGATGCAGTAGGC;
Asf1a Reverse:
(SEQ ID No 2)
CCTGGGATTAGATGCCAAAA;
Asf1b Forward:
(SEQ ID No 3)
CGAGTACCTCAACCCTGAGC;
Asf1b Reverse:
(SEQ ID No 4)
CCATGTTGTTGTCCCAGTTG;
RPLPO Forward:
(SEQ ID No 5)
GGCGACCTGGAAGTCCAACT;
RPLPO Reverse:
(SEQ ID No 6)
CCATCAGCACCACAGCCTTC;
HP1α Forward:
(SEQ ID No 11)
GATCATTGGGGCAACAGATT;
HP1α reverse:
(SEQ ID No 12)
TGCAAGAACCAGGTCAGCTT;
CAF-1 p60 Forward:
(SEQ ID No 17)
CGGACACTCCACCAAGTTCT;
CAF-1 p60 Reverse:
(SEQ ID No 18)
CCAGGCGTCTCTGACTGAAT;
CAF-1 p150 Forward:
(SEQ ID No 19)
CAGCAGTACCAGTCCCTTCC;
CAF-1 p150 Reverse:
(SEQ ID No 20)
TCTTTGCAGTCTGAGCTTGTTC;
GAPDH Forward:
(SEQ ID No 21)
GAGTCAACGGATTTGGTCGT;
GAPDH Reverse:
(SEQ ID No 22)
TTGATTTTGGAGGGATCTCG.

RNA Extraction and Quantitative RT-PCR: Breast Tumor Samples from 1995 and 1996

The RNeasy mini kit (QIAGEN) was used for total RNA extraction for transcriptome analysis and the miRNeasy mini kit (QIAGEN) for RNA extraction from frozen breast cancer samples (1995 and 1996) (De Koning et al., 2009). Reverse transcription and quantitative RT-PCR were performed as described below. All reverse transcription was performed using Superscript II reverse transcriptase (Invitrogen) with 500 ng-1 μg of RNA and 300 ng-3 μg of random primers (Invitrogen) per reaction respectively. For quantitative PCR analysis, the 96-well plate Step One Plus system (Applied Biosystems) was used and the SYBR Green PCR Master mix (Applied Biosystems) (transcriptome and breast tumor samples from 1995) or the QuantiTect SYBR Green RT-PCR kit (QIAGEN) or the SYBR® FAST ABI Prism® Master Mix (KAPA Biosystems) (breast tumor samples from 1996). Plates were filled using an EpMotion 5070 Robot (Eppendorf). Duplicates were measured in all experiments and the efficiency of each primer pair was checked with three subsequent cDNA dilutions for each of the breast tumor samples. For each gene, the quantity of mRNA was normalized to the quantity of mRNA corresponding to the human acidic ribosomal phosphoprotein PO (RPLPO) (de Cremoux et al., 2004) or to the Glyceraldehyde 3-phosphate dehydrogenase (GAPDH). To compare with the transcriptome analysis, mRNA levels relative to control siRNA levels were plotted and logarithmic 2 values were taken. This is referred to as the log2(fold change).

### Transcriptomic Data Analysis

In two independent experiments, mRNAs were prepared using U-2-OS cells treated with control, Asf1a, Asf1b or Asf1 (a+b) siRNAs for 48 h, and hybridized them on Affymetrix HG-U133-Plus2 oligonucleotide microarrays. The inventors determined differentially expressed genes using the Bioconductor package limma (Smyth, 2004). For each gene, they constructed a linear model that relates the expression value of the gene in the eight samples to a common intercept, an effect for the presence of the siRNA against Asf1a (siAsf1a) and an effect for the presence of the siRNA against Asf1b (siAsf1b). For each gene, a one-sample t-test was used to determine if the effects of siAsf1a and/or siAsf1b were significantly different from zero. For each test the variance of the gene term was shrunk towards an overall variance using the Empirical Bayes procedure of the Bioconductor package limma. The inventors corrected the significant p-values of the t-tests for multiple testing by controlling the False Discovery Rate (Benjamini et al., 2001). They report an effect as significantly different from zero if the corrected p-value of the test was less or equal 0.05. A Venn diagramm was drawn using the lists of differentially expressed genes (up- and down-regulated) determined against the control siRNA with a p-value of 0.05.

To investigate whether the resulting lists of differentially expressed genes had significant association with Gene Ontology terms (Ashburner et al., 2000), the inventors used the Bioconductor package topGO (Alexa et al., 2006). They obtained the Gene Ontology (GO) annotation of genes on the microarray from the Ensemble database in March 2009. They disregarded the associations between genes and GO terms which were solely inferred from electronic annotation (GO evidence terms: IEA, NAS, ND). To mitigate the dependencies between the tests imposed by the structure of the GO, if a gene was counted for the annotation of a specific gene, it was not counted again for any ancestor terms of this term (“elim” method of the package topGO). For each term, they performed a Hypergeometric test to determine whether genes of that list showed a more frequent association with a certain term than would be expected by chance given the GO annotation of all genes represented on the microarray. When the test resulted in a p-value inferior or equal to 0.001, they considered these terms as significantly over-represented for the given list. They used the statistical software R (2.5.0 version) to visualize gene expression values.

Breast Cancer Samples from 1995

Used samples from patients with breast tumor classified as non-palpable (T0) or small (T1-T2), lymph node negative (N0) and metastasis free (M0), were selected at the Institut Curie Biological Resources Center and treated with primary conservative tumorectomy. 92 patients diagnosed in 1995 granted permission to use their sample and data for research purposes. Table lA provides patients' and tumor characteristics. RNA extracted from 86 cryofrozen tissue of sufficient quality were selected for further analysis by RT-QPCR and carried out statistical analyses. The median follow-up of the patients was 146 months (range: 30-161 months). Recurrence-free and alive patients were censored to the date of their last known contact. At the date of the analysis, 11% of the patients were no longer alive, with cause of death being the initial breast cancer in 70% of these cases. 10% of patients developed loco-regional recurrence and 15% developed metastasis.

Breast Cancer Samples from 1996

Samples from patients with breast tumor classified as non-palpable (T0) or small (T1-T2), lymph node negative (N0) and metastasis free (M0), were selected at the Institut Curie Biological Resources Center and treated with primary conservative tumorectomy (median tumor size: 17 mm, range: 8-35 mm). 71 patients diagnosed in 1996 granted permission to use their sample and data for research purposes. Table 2 provides patients' and tumor characteristics. Recurrence-free and alive patients were censored to the date of their last known contact. At the date of the analysis, 17% of the patients were no longer alive, with cause of death being the initial breast cancer in 58% of these cases. 9% of patients developed loco-regional recurrence and 18% developed metastasis. RNA extracted from 71 cryo frozen tissue of sufficient quality were selected for further analysis by RT-QPCR.

Breast Tumor Samples from 1995 and 1996: Statistics

For each gene, the quantity x of the gene mRNA relative to the quantity of RPLPO mRNA was expressed in given sample by applying x=100/(Ê(Cp Gene—Cp RPLPO)), where E is the mean efficiency of the primers. For statistical analysis, data from 55 (1995) or 64 (1996) patients for Asf1a, and 85 (1995) or 69 (1996) patients for Asf1b, 75 (1995) or 70 patients (1996) for CAF-1 p60 and 86 (1995) or 71 (1996) patients for CAF-1 p150, which fulfilled the amplification criteria (reproducible duplicates, consistent primer efficiency between samples) were retained. Importantly, because of the difference in the number of patients with data for Asf1a and for Asf1b, it was verified that there was no significant differences in the composition of the two populations of patients (data not shown).

Correlations were calculated between various factors using the Pearson correlation coefficient method and analyzed differences between groups with the Kruskal-Wallis test for continuous variables. The disease-free interval is defined as the time from the diagnosis of breast cancer until the occurrence of disease progression, meaning local recurrence in the treated breast, regional recurrence in lymph node-bearing areas, controlateral breast cancer or distant recurrences. A cut-off value that is prognostic for the disease free interval (DFI) by using a Cox proportional risks model was determined and used the Wald test to evaluate the prognostic value of this variable on each event. The overall survival (OS), the metastasis-free interval and the DFI rates were estimated using the Kaplan-Meier method and compared the values between groups using a log-rank test. The inventors carried out a multivariate analysis to assess the relative influence of certain prognostic factors (age, number of mitosis, grade, estrogen and progesteron-receptor status as well as p60, p150, HP1a, Asf1b, Ki67 expression levels) on OS, DFI and metastasis free interval using the Cox stepwise forward procedure (Cox 1972). The significance level was 0.05. The statistical software R (2.5.0 version) was used for the analyses.

### Production of the Recombinant HIRA B-Domain Proteins and GST Pull-Down

The inventors carried out the induction with IPTG 0.4 mM at 30° C. during 3 h. Bacteria were centrifuged at 5 000 g 15 min at 4° C. and washed with cold water. Bacteria pellets were then frozen in liquid nitrogen and stored at −80° C. For the purification of recombinant proteins, the pellets were thawed on ice and resuspended in cold PBS containing protease inhibitor cocktail (Complete without EDTA, Roche Diagnostic) and 1 mg/ml lysosyme (Sigma Aldricht). After 10 min incubation on ice, bacteria suspensions were sonicated using a Branson sonifier. 1% (final) triton X100 was added to the extract that was incubated another 30 min on ice. Extracts were then cleared at 20 000 g for 15 min at 4° C. Supernatants were incubated with GST beads (GE Healthcare) equilibrated in PBS, protease inhibitor and Triton X100 (binding buffer) for 2 h at 4° C. on a wheel. Beads were washed in the binding buffer supplemented with 150 mM NaCl and stored at 4° C. or the GST recombinant proteins were eluted by adding 3 times some Tris HCl 50 mM pH8, 10 mM Gluthatione (Sigma Aldricht). Elutions were dialized against the same buffer without the gluthatione and the GST recombinant proteins were stored at −80° C.

GST pull-downs were performed by mixing 6 ml of High speed xenopus egg extract (HSE prepared as described in (Almouzni, 1998)), 1X nucleosome assembly buffer, 300 ng p^{bluescript}, 4 mM ATP, 100 mg/ml creatine kinase and 3 mg of recombinant protein on beads during 3 h at 23° C. The flow through was recovered and beads were washed 3 times with PBS supplemented with 150 mM NaCl and 0,5% NP40. Different fractions were loaded on a NuPAGE 4-12% (Invitrogen) and ran in MES buffer. Gels were transferred for 1 h, at 15 V on a nitrocellulose membrane (0.22 mm, Whatman) blocked with PBS, 0.1% tween20 and blotted 2 h at room temperature with specified antibody.

### Nucleosome Assembly Reaction

In vitro nucleosome assembly reactions independent of DNA synthesis were performed for 3 hours at 23° C. with 10 uL of HSE (Almouzni, 1998) and non-irradiated pBS plasmid as described in (Ray-Gallet and Almouzni, 2004). For the dominant negative assay, 2, 4 or 8 mg of the recombinant protein were added directly in the nucleosome assembly reaction mix without any prior incubation. At the end of the assembly reaction, a small aliquot was removed for analysis by western blotting. The supercoiling of the purified DNA plasmid was examined by agarose gel electrophoresis in 1× TAE buffer and visualized by staining with ethidium bromide (Ray-Gallet and Almouzni, 2004).

### Example 2

Materials and methods

### RNA Extraction and QPCR Analysis

RNA extraction and QPCR analysis were performed as disclosed in example 1.

### Primers

For analysis of the 122 ovarian tumor samples, the following primers were used:

Asf1a Forward:
(SEQ ID No 1)
CAGATGCAGATGCAGTAGGC;
Asf1a Reverse:
(SEQ ID No 2)
CCTGGGATTAGATGCCAAAA;
Asf1b Forward:
(SEQ ID No 3)
CGAGTACCTCAACCCTGAGC;
Asf1b Reverse:
(SEQ ID No 4)
CCATGTTGTTGTCCCAGTTG;
CAF-1 p60 Forward:
(SEQ ID No 17)
CGGACACTCCACCAAGTTCT;
CAF-1 p60 Reverse:
(SEQ ID No 18)
CCAGGCGTCTCTGACTGAAT;
Mcm2 Forward:
(SEQ ID No 31)
ACCAGGACAGAACCAGCATC;
Mcm2 Reverse:
(SEQ ID No 32)
CAGGATGTCAAAGCGTGAGA;
HJURP Forward:
(SEQ ID No 33)
GCTGGAAGGGATGTACGTGT;
HJURP Reverse:
(SEQ ID No 34)
TGGGTCACCAGGACTCTTTC;
RPLPO Forward:
(SEQ ID No 35)
AACTCTGCATTCTCGCTTCC;
RPLPO Reverse:
(SEQ ID No 36)
TCGTTTGTACCCGTTGATGA.

### Ovarian Tumor Samples and Statistics

The inventors used samples from patients of 1989 to 2008 with ovarian tumor selected at the Institut Curie Biological Resources Center and treated with primary conservative tumorectomy. 122 patients granted permission to use their sample and data for research purposes. Recurrence-free and alive patients were censored to the date of their last known contact. At the date of the analysis, 38% of the patients were no longer alive, with cause of death being the initial ovarian cancer in 94% of these cases. 44% of patients developed loco-regional recurrence and 70% developed metastasis. Patients and tumor characteristics are summarized in Table 4 below

TABLE 4
Description of the samples from patients with ovarian cancer

| Age | Median: 58 (Range: 31-86) | | Histological | I | 5% | Chemotherapy | No | 12% |
|---|---|---|---|---|---|---|---|---|
| Menopaused | Yes: | 23% | grade | II | 32% | | Yes | 88% |
| | No: | 77% | | III | 63% | Recidivism | No | 56% |
| Histological | serious | 74% | Histological | I | 17% | | Yes | 44% |
| type | mucinous | 5% | stage | II | 11% | Metastasis | No | 30% |
| | endometrioid | 11% | | IIIa | 7% | | Yes | 70% |
| | other | 11% | | IIIb-IV | 66% | | | |

The inventors selected RNA extracted from 122 cryofrozen tissue of sufficient quality for analysis by RT-QPCR. For each gene, the quantity x of the gene mRNA was expressed relative to the quantity of RPLPO mRNA in a given sample by applying x=100*(Ê(Cp RPLPO—Cp Gene)), where E is the mean efficiency of the primers. For statistical analysis, the inventors retained data from all 122 patients for Asf1a, Asf1b, CAF-1 p60, Mcm2 and HJURP which fulfilled their amplification criteria (reproducible duplicates, consistent primer efficiency between samples).

Correlations between various factors were calculated using the Pearson correlation coefficient method and differences between groups were analysed with the Kruskal-Wallis test for continous variables. The significance level was 0.05. The statistical software R (2.5.0 version) was used for these analyses.

### Results

Asf1b Correlates with Proliferation in Ovarian Tumor Samples

To assess the relevance of findings connecting Asf1b with proliferation in a physiological context, the inventors analysed a selection of cryopreserved ovarian carcinoma samples collected between 1898 and 2008 at the Institut Curie. Table 4 provides the patients' and tumor characteristics. The standard treatment received by the patients at the Institut Curie for ovarian cancers was tumor excision with 88% of patients receiving chimiotherapy. Asf1a, Asf1b, CAF-1 p60, Mcm2 and HJURP mRNA expression levels were measured by quantitative RT-PCR in 122 ovarian tumor samples and the expression levels were normalized to the known reference gene RPLPO (de Cremoux et al., 2004). For statistical analysis, only data that fulfilled amplification quality criteria were kept.

First, the correlation between the levels of Asf1 iso forms and that of other proliferation markers such as CAF-1 p60 (Polo et al., 2004) or HJURP and Mcm2 was studied. Asf1a levels only weakly correlated with that of Asf1b, CAF-1 p60, Mcm2 and HJURP (FIG. 23 and data not shown). In contrast, Asf1b levels significantly correlated with p60 (r=0.7; p<10⁻¹⁶), Mcm2 (r=0.8; p<10⁻²⁸) and HJURP (r=0.8; p<10⁻³¹), which again demonstrated its important link with cell proliferation (FIG. 23). The inventors then investigated the correlation of Asf1b levels with clinical parameters to evaluate a potential diagnostic value. They found a high significant positive correlation of Asf1b levels with the histological grade of the tumor (p=2e-04), the histological stage of the tumor (p=0.043), the tumor resection (p=0.035) and the family antecedents of breast cancer (p=0.049) (FIG. 24). Taken together, these observations put forward Asf1b as a new proliferation marker of clinical interest in the context of ovarian cancer.

### REFERENCES

Alexa et al., 2006, Bioinformatics 22, 1600-7

Almouzni, G. 1998. Assembly of chromatin and nuclear structures in Xenopus egg extracts. H Gould, Editor, Chromatin: A Practical Approach, Oxford University Press, Oxford, 195-218.

Ashburner et al., 2000, Nat Genet. 25, 25-9.

Barretina et al. 2010. Nat Genet 42, 715-721.

Benjamini et al., 2001, Behav Brain Res. 125, 279-84.

Bernstein, et al., 2001, Nature 409, 363-6.

Carroll et al., 2000, J Biol Chem 275, 38221-38229.

Corpet et al., 2009, Trends Cell Biol 19, 29-41.

Corpet and Almouzi, 2009, EMBO J 28, 1828-30.

Daganzo et al., 2003, Curr Biol 13, 2148-2158.

Das et al., 2009, Nature 459, 113-119.

de Cremoux et al., 2004, Endocr Relat Cancer 11, 489-495.

De Koning et al., 2007, Nat Struct Mol Bio114, 997-1007.

De Koning et al., 2009, EMBO Mol Med 1, 178-191.

D'Errico et al. 2009. Eur J Cancer 45, 461-469.

Elbashir et al., 2001a, Embo J 20, 6877-88.

Elbashir et al., 2001b, Nature 411, 494-8.

Elston and Ellis, 2002, Histopathology 41, 151-3.

English et al, 2006, Cell 127, 495-508.

Fanning and Symonds, 2006, RNA Towards Medicine (Handbook of Experimental Pharmacology), ed. Springer p. 289-303

Franken et al., 2006, Nat Protoc 1, 2315-2319.

Groth et al., 2005, Mol Cell 17, 301-311

Groth et al., 2007, Science 318, 1928-1931.

Hackett et al., 1977, J Natl Cancer Inst 58, 1795-1806.

Hu et al., 2010, Breast Cancer Res. 12, R18.

Jasencakova et al., 2010, Mol. Cell. 37, 736-43.

Kurdistani, 2007, Br J Cancer 97, 1-5.

Malay et al., 2008, J Biol Chem 283, 14022-31.

Martini et al., 1998, J Cell Bio1143, 563-575.

Mello et al. 2002. EMBO Rep 3, 329-334.

Moggs et al. 2000. Mol Cell Biol 20, 1206-1218.

Mousson et al. 2005. Proc Natl Acad Sci USA 102, 5975-5980.

Natsume et al., 2007, Nature 446, 338-341.

Osborne et al., 1997, Curr Opin Chem Biol. 1, 5-9.

Pei et al. 2009. Cancer Cell 16, 259-266.

Polo et al., 2004, Cancer Res 64, 2371-2381.

Pilyugin et al 2009. PLoS ONE 4, e8328.

Quivy et al. 2001. Embo J 20, 2015-2027.Ransom et al., 2010, Cell 140, 183-195.

Ray-Gallet et al. 2002. Mol Cell 9, 1091-1100.

Ray-Gallet et al., 2004, Methods Enzymol 375, 117-131

Reyal et al., 2008, Breast Cancer Research 10, R93

Rhodes et al., 2004, Neoplasia 6, 1-6.

Rosty et al., 2005, Oncogene 24, 7094-7104.

Sabates-Bellver et al. 2007 Mol Cancer Res 5, 1263-1275

Sanchez-Carbayo et al. 2006. J Clin Oncol 24, 778-789

Santegoets et al. 2007. Int J Cancer 121, 759-766

Schonk et al., 1989, Hum Genet 83, 297-299.

Smyth, 2004, Stat Appl Genet Mol Biol 3, Article3.

Sotiriou et al., 2006, J Natl Cancer Inst 98, 262-272.

Sotiriou and Piccart, 2007, J Natl Cancer Inst 98, 262-272.

Sotirou and Pusztai, 2009, N Engl J Med 360:790.

Sun et al. 2006. Cancer Cell 9, 287-300

Tamburini et al., 2005, Eukaryot Cell 4, 1583-1590.

Tang et al., 2006, Nat Struct Mol Biol 13, 921-929. Epub 2006 September 2017.

Umehara et al, 2003, J Biol Chem 278, 35660-35667.

van 't Veer et al., 2002, Nature 415, 530-536.

Wirapati et al., 2008, Breast Cancer Res 10, R65.

Yuan et al., 2009, Cell Cycle 8, 1747-53.

Zamore et al., 2000, Cell 101, 25-33.

Zhang et al., 2005, Dev Cell 8, 19-30.

1-28. (canceled)

29. An in vitro method for predicting or monitoring clinical outcome of a subject affected with a cancer, wherein the method comprises the step of determining the expression level of Asf1b (anti-silencing function 1b) in a cancer sample from said subject, a high expression level of Asf1b being indicative of a poor clinical outcome for said subject.

30. The method according to claim 29, said method further comprising the administration of a cancer therapy, preferably an adjuvant therapy, to a subject having a high expression level of Asf1b.

31. The method according to claim 29, wherein the expression level of Asf1b is determined by measuring the quantity of Asf1b protein or Asf1b mRNA.

32. The method according to claim 31, wherein the quantity of Asf1b protein is measured by immunohistochemistry, semi-quantitative Western-Blot, or protein or antibody arrays.

33. The method according to claim 31, wherein the quantity of Asf1b mRNA is measured by quantitative or semi-quantitative RT-PCR, or by real time quantitative or semi-quantitative RT-PCR or by transcriptome approaches.

34. The method according to claim 29, wherein the method further comprises the step of comparing the expression level of Asf1b to a reference expression level of Asf1B.

35. The method according to claim 29, wherein a poor clinical outcome for said subject is decreased patient survival and/or an early disease progression and/or an increased disease recurrence and/or increased metastasis formation.

36. The method according to claim 29, said method further comprising assessing at least one another cancer or prognosis marker selected from tumor grade, hormone receptor status, mitotic index, tumor size, HJURP expression level or expression of proliferation markers selected from Ki67, MCM2, CAF-1 p60 or CAF-1 p150 or a prognosis marker.

37. The method according to claim 29, wherein the cancer is selected from the group consisting of breast cancer, osteosarcoma, skin cancer, ovarian cancer, lung cancer, liver cancer, cervix cancer, liposarcoma, gastric cancer, pancreatic cancer, bladder cancer, vulvar cancer, colon cancer and brain cancer.

38. The method according to claim 37, wherein the cancer is breast cancer or early stage breast cancer without local or systemic invasion.

39. An in vitro method for monitoring the response to a treatment of a subject affected with a cancer, wherein the method comprises determining the expression level of Asf1b in a cancer sample from said subject before the administration of the treatment, and in a cancer sample from said subject after the administration of the treatment, a decreased expression level of Asf1b in the sample obtained after the administration of the treatment indicating that the subject is responsive to the treatment.

40. A method for treating a cancer by administering a therapeutic effective amount of a compound inhibiting the Asf1b.

41. The method according to claim 40, wherein the compound is selected from the group consisting of a small molecule, an aptamer, an antibody, a nucleic acid and a molecule preventing the interaction Asf1b with an Asf1b interacting partner.

42. The method according to claim 40, wherein the molecule is selected from the group consisting of an antibody against Asf1b and a nucleic acid molecule interfering specifically with Asf1b expression.

43. The method according to claim 42, wherein the molecule is a nucleic acid molecule interfering specifically with Asf1b expression and is selected from the group consisting of an antisense against Asf1b and a siRNA against Asf1b.

44. The method according to claim 42, wherein the antibody, antisense or siRNA is specific of Asf1b in comparison to Asf1a.

45. The method according to claim 40, wherein, the compound is a fragment of the B-domain of HIRA or is a fragment of the B-domain of HIRA comprising SEQ ID NO: 28.

46. The method according to claim 40, wherein the administration of a therapeutic effective amount of a compound inhibiting the Asf1b is combined with radiotherapy or a treatment with an anti-tumoral agent.

47. A method for selecting or identifying a molecule useful for the treatment of cancer comprising testing a molecule for its ability to inhibit Asf1b interaction with HIRA or CAF-1 p60 and selecting the molecule capable of inhibiting Asf1b interaction with HIRA or CAF-1 p60.

48. A combined preparation product or kit containing (a) a compound inhibiting Asf1b and (b) an anti-tumoral agent as a combined preparation for simultaneous, separate or sequential use in the treatment of cancer with a high expression level of Asf1b.


SEQUENCE LISTING (36 sequences)

SEQ ID NO: 1 - DNA, 20 bp, artificial sequence
  Primer
cagatgcagatgcagtaggc

SEQ ID NO: 2 - DNA, 20 bp, artificial sequence
  primer
cctgggattagatgccaaaa

SEQ ID NO: 3 - DNA, 20 bp, artificial sequence
  primer
cgagtacctcaaccctgagc

SEQ ID NO: 4 - DNA, 20 bp, artificial sequence
  primer
ccatgttgttgtcccagttg

SEQ ID NO: 5 - DNA, 20 bp, artificial sequence
  primer
ggcgacctggaagtccaact

SEQ ID NO: 6 - DNA, 20 bp, artificial sequence
  primer
ccatcagcaccacagccttc

SEQ ID NO: 7 - protein, 204 aa, homo sapiens
MAKVQVNNVVVLDNPSPFYNPFQFEITFECIEDLSEDLEWKIIYVGSAESEEYDQVLDSVLVGPVPAGRHMFVFQADAPNPGLIPDADAVGVTVVLITCTYRGQEFIRVGYYVNNEYTETELRENPPVKPDFSKLQRNILASNPRVTRFHINWEDNTEKLEDAESSNPNLQSLLSTDALPSASKGWSTSENSLNVMLESHMDCM

SEQ ID NO: 8 - protein, 202 aa, Homo sapiens
MAKVSVLNVAVLENPSPFHSPFRFEISFECSEALADDLEWKIIYVGSAESEEFDQILDSVLVGPVPAGRHMFVFQADAPNPSLIPETDAVGVTVVLITCTYHGQEFIRVGYYVNNEYLNPELRENPPMKPDFSQLQRNILASNPRVTRFHINWDNNMDRLEAIETQDPSLGCGLPLNCTPIKGLGLPGCIPGLLPENSMDCI

SEQ ID NO: 9 - DNA, 21 bp, artificial sequence
  primer
attgaacctgcggaagagctg

SEQ ID NO: 10 - DNA, 21 bp, artificial sequence
  primer
ggagcgcagggatattccctt

SEQ ID NO: 11 - DNA, 20 bp, artificial sequence
  primer
gatcattggggcaacagatt

SEQ ID NO: 12 - DNA, 20 bp, artificial sequence
  primer
tgcaagaaccaggtcagctt

SEQ ID NO: 13 - DNA, 21 bp, artificial sequence
  siRNA
gtgaagaatacgatcaagttt

SEQ ID NO: 14 - DNA, 21 bp, artificial sequence
  siRNA
caacgagtacctcaacccttt

SEQ ID NO: 15 - DNA, 21 bp, artificial sequence
  siRNA
gcgcgctttgtaggattcgtt

SEQ ID NO: 16 - DNA, 21 bp, artificial sequence
  siRNA
cacttgtcactactttctctt

SEQ ID NO: 17 - DNA, 20 bp, artificial sequence
  primer
cggacactccaccaagttct

SEQ ID NO: 18 - DNA, 20 bp, artificial sequence
  primer
ccaggcgtctctgactgaat

SEQ ID NO: 19 - DNA, 20 bp, artificial sequence
  primer
cagcagtaccagtcccttcc

SEQ ID NO: 20 - DNA, 22 bp, artificial sequence
  primer
tctttgcagtctgagcttgttc

SEQ ID NO: 21 - DNA, 20 bp, artificial sequence
  primer
gagtcaacggatttggtcgt

SEQ ID NO: 22 - DNA, 20 bp, artificial sequence
  primer
ttgattttggagggatctcg

SEQ ID NO: 23 - DNA, 39 bp, Artificial
  primer
aggtgctagaattcaacatggacaggctggaggccatag

SEQ ID NO: 24 - DNA, 43 bp, Artificial
  primer
gactcttgaggtacctgacgtagattattgagctctatcggac

SEQ ID NO: 25 - DNA, 36 bp, Artificial
  primer
aggtgctagaattcaacacagaaaaactggaagatg

SEQ ID NO: 26 - DNA, 41 bp, Artificial
  primer
cttagggtgtacctgacgtacactattgagctctatcggac

SEQ ID NO: 27 - DNA, 138 bp, Xenopus laevis
ggggaaagcttggaggacataagaaagaacctcttgaaaaagcaagtggagacacgaacagctgatggacggcgaaggatcactccactctgcattgctcagctagacactggggacttttccacagcgtttttcaat

SEQ ID NO: 28 - protein, 46 aa, Xenopus laevis
GESLEDIRKNLLKKQVETRTADGRRRITPLCIAQLDTGDFSTAFFN

SEQ ID NO: 29 - DNA, 138 bp, Artificial
  DNA sequence of the mutated HIRA B-Domain I461D
ggggaaagcttggaggacataagaaagaacctcttgaaaaagcaagtggagacacgaacagctgatggacggcgaagggacactccactctgcattgctcagctagacactggggacttttccacagcgtttttcaat

SEQ ID NO: 30 - protein, 46 aa, Artificial
  Mutated HIRA B-Domain I461D protein
GESLEDIRKNLLKKQVETRTADGRRRDTPLCIAQLDTGDFSTAFFN

SEQ ID NO: 31 - DNA, 20 bp, Artificial
  Primer
accaggacagaaccagcatc

SEQ ID NO: 32 - DNA, 20 bp, artificial
  Primer
caggatgtcaaagcgtgaga

SEQ ID NO: 33 - DNA, 20 bp, Artificial
  Primer
gctggaagggatgtacgtgt

SEQ ID NO: 34 - DNA, 20 bp, Artificial
  Primer
tgggtcaccaggactctttc

SEQ ID NO: 35 - DNA, 20 bp, Artificial
  Primer
aactctgcattctcgcttcc

SEQ ID NO: 36 - DNA, 20 bp, Artificial
  Primer
tcgtttgtacccgttgatga